(19) United States
(12) Patent Application Publication
(10) Pub. No.: US 2024/0252633 A1
(43) Pub. Date: Aug. 1, 2024

(54) METHODS OF TREATING CANCER WITH CD-40 AGONISTS

(71) Applicant: Apexigen America, Inc., Boston, MA (US)

(72) Inventors: Lacey Padron, San Francisco, CA (US); Theresa Lavallee, San Francisco, CA (US); Pier Federico Gherardini, San Francisco, CA (US); Deena Maurer, San Francisco, CA (US)

(73) Assignee: Apexigen America, Inc., Boston, MA (US)

(21) Appl. No.: 18/565,446

(22) PCT Filed: Jun. 2, 2022
(86) PCT No.: PCT/US2022/032010

§ 371 Date: Nov. 29, 2023

Related U.S. Application Data
(60) Provisional application No. 63/196,676, filed on Jun. 3, 2021.

(51) Int. Cl. A61K 39/395; A61K 31/513; A61K 47/64; A61P 35/00; C12Q 1/6886; G01N 33/574

(52) U.S. Cl. CPC A61K 39/3955; A61K 31/513; A61K 47/643; A61P 35/00; C12Q 1/6886; G01N 33/57484; C12Q 2600/106; C12Q 2600/158

(57) ABSTRACT

The present disclosure provides methods of identifying a sub-population of cancer patients amendable for a combination therapy with a CD40 agonist and one or more chemotherapy drugs and treating the sub-population of cancer patients with the combination therapy.

### CROSS-REFERENCE TO RELATED APPLICATIONS

This application is a U.S. National Stage application under 35 U.S.C. § 371 of International Application No. PCT/US2022/032010, having an international filing date of Jun. 2, 2022, and published in English, which claims the benefit of U.S. Provisional Patent Application No. 63/196,676, filed Jun. 3, 2021, all of which are incorporated herein by reference in their entireties for all purposes.

### FIELD

The present disclosure relates to methods of identifying a sub-population of cancer patients amenable for a combination therapy with a CD40 agonist and one or more chemotherapy drugs.

### BACKGROUND

Complete T cell activation requires two separate but synergistic signals. The first signal coming through the T-cell antigen receptor is provided by the antigen and MHC complex at the APC and is responsible for the specificity of the immune response. The secondary or co-stimulating signal comes through the interaction of CD28 with B7-1 (CD80)/B7-2 (CD86) and CD40 with CD40L, which are required for the implementation of a full T-cell response. In the absence of co-stimulating signals, T cells during antigenic stimulation may become immune (anergy) or enter programmed cell death (apoptosis).

CD40, a member of the TNF receptor superfamily (TNFR), is expressed predominantly on B cells and other antigen presenting cells (APCs), such as dendritic cells and macrophages. The CD40 ligand (CD40L) is expressed primarily by activated T cells.

The interaction of CD40 and CD40L serves as a co-stimulating signal for the activation of T cells. The formation of the CD40-CD40L complex on resting cells induces proliferation, immunoglobulin class switching, antibody secretion, and also plays a role in the development of germinal centers and the survival of memory B cells, which are all important for the humoral immune response. The binding of CD40L to CD40 on dendritic cells (DC) induces DC maturation, as evidenced by an increase in the expression of co-stimulating molecules, such as the B7 family of molecules (CD80, CD86), and an increase in the production of pro-inflammatory cytokines, such as interleukin 12. This leads to strong T-cell response.

CD40 signaling activates several pathways, including NFκB (nuclear factor kV), MAPK (mitogen-activated protein kinase), and STAT3 (signal transducer and transcription activator-3) that regulate gene expression by activating proteins, c-Jun, ATF2 (transcription activation factor-2) and transcription factors Rel. Adaptive proteins, factors associated with the TNF receptor (TNFR) (e.g., TRAF1, TRAF2, TRAF3, TRAF5 and TRAF6), interact with this receptor and mediate signal transduction. Depending on the specific cell type, activation of CD40 leads to the expression of a specific set of genes. Genes activated in response to signal transmission from CD40 include numerous cytokines and chemokines (IL-1, IL-6, IL-8, IL-10, IL-12, TNF-alpha and macrophage-1 inflammatory protein alpha (MIP1α)) In some cell types, activation of CD40 can lead to the production of cytotoxic radicals (COX-2 (cyclooxygenase-2), and NO (nitric oxide) production.

CD40 is overexpressed in a wide range of malignant cells. The role of CD40 in inhibiting tumors and stimulating the immune system makes CD40 an attractive target for antibody-based immunotherapy. Anti-CD40 antibodies can act against tumor cells through several mechanisms: (i) the effector function of antibodies, such as ADCC, (ii) the direct cytotoxic effect on tumor cells and (iii) the activation of the antitumor immune response. However, there is a significant need to develop methods to identify a subset of patients who may experience remarkable clinical benefit with anti-CD40 treatment.

### SUMMARY

In at least one embodiment, the present disclosure provides a method comprising: (a) determining a MYC gene signature from a test biological sample from the subject and one or more reference biological samples, wherein a reference biological sample of the one or more reference biological samples is collected from each individual among a cohort of subjects having the same cancer, wherein the subject is part of the cohort; (b) calculating a MYC gene signature score of the subject; (c) calculating a MYC gene signature score of the cohort; and (d) aiding the treatment of the subject with a combination of an anti-CD40 therapy and chemotherapy when the MYC gene signature score of the subject is lower than the MYC gene signature score of the cohort.

In at least one embodiment, the present disclosure provides a method comprising: (a) determining a MYC gene signature of a test biological sample and one or more reference biological samples, wherein a reference biological sample of the one or more reference biological samples is collected from each individual among a cohort of subjects having the same cancer, wherein the subject is part of the cohort; (b) calculating a MYC gene signature score of the subject; (c) calculating a MYC gene signature score of the cohort; (d) treating the subject with a combination of an anti-CD40 therapy and chemotherapy, wherein the MYC gene signature score of the subject is lower than the MYC gene signature score of the cohort.

In at least one embodiment, the MYC gene signature score is calculated by averaging log normalized expression values for each gene in a MYC gene set. In at least one embodiment, the MYC gene set comprises one or more of genes known to be regulated by MYC version 1 (V1). In at least one embodiment, the one or more genes are selected from the group consisting of tumor suppressor genes, oncogenes, translocated cancer genes, protein kinase genes, cell differentiation marker genes, homeodomain protein genes, transcription factor genes, cytokine genes, and growth factor genes. In at least one embodiment, the one or more genes are selected from the group consisting of ABCE1, ACP1, AIMP2, AP3S1, APEX1, BUB3, C1QBP, CAD, CANX, CBX3, CCNA2, CCT2, CCT3, CCT4, CCT5, CCT7, CDC20, CDC45, CDK2, CDK4, CLNS1A, CNBP, COPS5, COX5A, CSTF2, CTPS1, CUL1, CYC1, DDX18, DDX21, DEK, DHX15, DUT, EEF1B2, EIF1AX, EIF2S1, EIF2S2, EIF3B, EIF3D, EIF3J, EIF4A1, EIF4E, EIF4G2, EIF4H, EPRS1, ERH, ETF1, EXOSC7, FAM120A, FBL, G3BP1, GLO1, GNL3, GOT2, GSPT1, H2AZ1, HDAC2, HDDC2, HDGF, HNRNPA1, HNRNPA2B1, HNRNPA3, HNRNPC, HNRNPD, HNRNPR, HNRNPU, HPRT1, HSP90AB1, HSPD1, HSPE1, IARS1, IFRD1, ILF2, IMPDH2, KARS1, KPNA2, KPNB1, LDHA, LSM2, LSM7, MAD2L1, MCM2, MCM4, MCM5, MCM6, MCM7, MRPL23, MRPL9, MRPS18B, MYC, NAP1L1, NCBP1, NCBP2, NDUFAB1, NHP2, NME1, NOLC1, NOP16, NOP56, NPM1, ODC1, ORC2, PA2G4, PABPC1, PABPC4, PCBP1, PCNA, PGK1, PHB, PHB2, POLD2, POLE3, PPIA, PPM1G, PRDX3, PRDX4, PRPF31, PRPS2, PSMA1, PSMA2, PSMA4, PSMA6, PSMA7, PSMB2, PSMB3, PSMC4, PSMC6, PSMD1, PSMD14, PSMD3, PSMD7, PSMD8, PTGES3, PWP1, RACK1, RAD23B, RAN, RANBP1, RFC4, RNPS1, RPL14, RPL18, RPL22, RPL34, RPL6, RPLP0, RPS10, RPS2, RPS3, RPS5, RPS6, RRM1, RRP9, RSL1D1, RUVBL2, SERBP1, SET, SF3A1, SF3B3, SLC25A3, SMARCC1, SNRPA, SNRPA1, SNRPB2, SNRPD1, SNRPD2, SNRPD3, SNRPG, SRM, SRPK1, SRSF1, SRSF2, SRSF3, SRSF7, SSB, SSBP1, STARD7, SYNCRIP, TARDBP, TCP1, TFDP1, TOMM70, TRA2B, TRIM28, TUFM, TXNL4A, TYMS, U2AF1, UBA2, UBE2E1, UBE2L3, USP1, VBP1, VDAC1, VDAC3, XPO1, XPOT, XRCC6, YWHAE, and YWHAQ.

In at least one embodiment, the present disclosure provides a method comprising: (a) counting circulating CD244⁺ effector memory CD4⁺ T cells and total effector memory CD4⁺ T cells in a test biological sample and one or more reference biological samples, wherein a reference biological sample of the one or more reference biological samples is collected from each individual among a cohort of subjects having the same cancer, wherein the subject is part of the cohort; (b) determining a first ratio of a first number of the circulating CD244⁺ effector memory CD4⁺ T cells to a second number of the total effector memory CD4⁺ T cells in the test biological sample; (c) determining a second ratio of a third number of the circulating CD244⁺ effector memory CD8⁺ T cells to a fourth number of total effector memory CD4⁺ T cells in the one or more reference biological samples; and (d) treating the subject with a combination of an anti-CD40 therapy and chemotherapy, wherein the first ratio in (c) of the subject is lower than the second ratio in (d) of the cohort.

In at least one embodiment, the effector memory CD4⁺ T cells are CD45RA⁻CD27⁻.

In at least one embodiment, the present disclosure provides a method of treating a subject with cancer comprising: (a) counting circulating CXCR5⁺ effector memory CD8⁺ T cells and total effector memory CD8⁺ T cells in a test biological sample and one or more reference biological samples, wherein a reference biological sample of the one or more reference biological samples is collected from each individual among a cohort of subjects having the same cancer, wherein the subject is part of the cohort; (b) determining a first ratio of a first number of the circulating CXCR5⁺ effector memory CD8⁺ T cells to a second number of the total effector memory CD8⁺ T cells in the test biological sample; (c) determining a second ratio of a third number of the circulating CXCR5⁺ effector memory CD8⁺ T cells to a fourth number of total effector memory CD8⁺ T cells in the one or more reference biological samples; and (d) treating the subject with a combination of an anti-CD40 therapy and chemotherapy, wherein the first ratio in (c) of the subject is lower than the second ratio in (d) of the cohort.

In at least one embodiment, the effector memory CD8⁺ T cells are CD45RA-CD27⁺. In at least one embodiment, the test biological sample and the one or more reference biological samples is a tumor sample.

In at least one embodiment, the test biological sample and the one or more reference biological samples is a blood sample. In at least one embodiment, peripheral blood mononuclear cells (PBMCs) are isolated from the blood.

In at least one embodiment, the test biological sample and the one or more reference biological samples are obtained before initiation of any cancer treatment.

In at least one embodiment, the cancer is selected from the group consisting of a pancreatic cancer, an endometrial cancer, a non-small cell lung cancer (NSCLC), a renal cell carcinoma, a urothelial cancer, a head and neck cancer, a melanoma, a bladder cancer, a hepatocellular carcinoma, a breast cancer, an ovarian cancer, a gastric cancer, a colorectal cancer, a glioblastoma, a biliary tract cancer, a glioma, Merkel cell carcinoma, Hodgkin lymphoma, non-Hodgkin lymphoma, a cervical cancer, an advanced or refractory solid tumor, a small cell lung cancer, a non-squamous non-small cell lung cancer, desmoplastic melanoma, a pediatric advanced solid tumor or lymphoma, a mesothelin-positive pleural mesothelioma, an esophageal cancer, an anal cancer, a salivary cancer, a prostate cancer, a carcinoid tumor, a primitive neuroectodermal tumor (pNET), and a thyroid cancer. In at least one embodiment, the cancer is a pancreatic cancer.

In at least one embodiment, the anti-CD40 therapy comprises an anti-CD40 antibody or antigen binding fragment thereof. In at least one embodiment, the anti-CD40 antibody or antigen binding fragment thereof is selected from the group consisting of sotigalimab, selicrelumab, ChiLob7/4. ADC-1013, SEA-CD40, CP-870,893, dacctuzumab, and CDX-1140. In at least one embodiment, the anti-CD40 antibody is sotigalimab.

In at least one embodiment, the chemotherapy is selected from the group consisting of gemcitabine, nab-paclitaxel, folfirionx, nitrogen mustard/oxazaphosphorine, nitrosourca, triazene, and alkyl sulfonates, anthracycline antibiotics such as doxorubicin and daunorubicin, taxanes such as Taxol brand and docetaxel, vinca alkaloids such as vincristine and vinblastine, 5-fluorouracil (5-FU), leucovorin, Irinotecan, idarubicin, mitomycin C, oxaliplatin, raltitrexed, pemetrexed, tamoxifen, cisplatin, carboplatin, methotrexate, a Tinomycin D, mitoxantrone, brenoxane, mitramycin, methotrexate, paclitaxel, 2-methoxyestradiol, purinomastert, batimastat, BAY 12-9656, carboxamidotriazole, CC-1088, dextromethorphan acetic acid, dimethylxanthenone acetic acid, Endostatin, IM-862, marimastat, penicillamine, PTK787/ZK 222584, RPI. 4610, squalamine lactate, SU5416, thalidomide, combretastatin, tamoxifen, COL-3, ncobasstat, BMS-275291, SU6668, anti-VEGF antibody, Med-522 (Vitaxin II), CAI, interleukin 12, IM862, amiloride, Angiostatin, angiostatin K1-3, angiostatin K1-5, captopril, DL-α-difluoromethylornithine, DL-α-difluoromethylornithine HCl, endostatin, fumagillin, herbimycin A, 4-hydroxyphenylretinamide, Juglone, laminin, laminin hexapeptide, laminin pentapeptide, labendustin A, medroxyprogesterone, minocycline, placental ribonuclease Inhibitors, suramin, thrombospondin, antibodies targeting pro-angiogenic factors, topoisomerase inhibitors, microtubule inhibitors, low-molecular-weight tyrosine kinase inhibitors of pro-angiogenic growth factors Agents, GTPase inhibitors, histone deacetylase inhibitors, AKT kinase or ATPase inhibitors, Win (Wnt) signal inhibitors, E2F transcription factor inhibitors, mTOR inhibitors Agents, α, β and γ interferons, IL-12, matrix metalloproteinase inhibitors, ZD6474, SU1248, vitaxin, PDGFR inhibitors, NM3 and 2-ME2, and sirengitide. In at least one embodiment, the chemotherapy is a combination of gemcitabine and nab-paclitaxel.

In at least one embodiment, the present disclosure provides a system comprising: reagents capable of binding to genes that are involved in MYC signaling; reagents capable of determining the ratio of circulating CD244⁺ effector memory CD4⁺ T cells to total effector memory CD4⁺ T cells; and reagents capable of determining the ratio of circulating CXCR5⁺ effector memory CD8⁺ T cells to total effector memory CD8⁺ T cells.

The present disclosure also provides a method of treating a cancer in a human subject in need thereof. The method involves (a) determining levels (cell counts) of circulating cross-presenting dendritic cells (DCs) in a biological sample from the subject; and (b) administering a CD40 agonist in combination with a chemotherapeutic agent to the subject if the levels (cell counts) of circulating cross-presenting DCs are increased relative to a control or reference.

In at least one embodiment, the cross-presenting DCs are CD1C+CD141+ and (a) comprises determining levels (cell counts) of CD1C+CD141+ DCs in the subject; and (b) comprises administering the CD40 agonist in combination with the chemotherapeutic agent to the subject if the levels (cell counts) of CD1C+CD141+ DCs are increased relative to the control or reference.

The present disclosure also provides a method of treating a cancer in a human subject in need thereof. The method involves (a) determining levels (cell counts) of circulating HLA−DR+CCR7+B cells in a biological sample from the subject; and (b) administering a CD40 agonist in combination with a chemotherapeutic agent to the subject if the levels (cell counts) of circulating HLA-DR+CCR7+B cells are increased relative to a control or reference.

The present disclosure further provides a method of treating a cancer in a human subject in need thereof. The method involves (a) determining levels (cell counts) of at least one of circulating PD-1+ T cells, circulating TCF-1+ T cells, and/or circulating Tbet+ T cells in a biological sample from the subject; and (b) administering a CD40 agonist in combination with a chemotherapeutic agent to the subject if the levels (cell counts) of at least one of the circulating PD-1+ T cells, circulating TCF-1+ T cells, and/or circulating Tbet+ T cells are increased relative to a control or reference.

Also provided is a method of treating a cancer in a human subject in need thereof. The method includes (a) determining levels (cell counts) of circulating 2B4+ CD4 T cells in a biological sample from the subject; and (b) administering a CD40 agonist in combination with a chemotherapeutic agent to the subject if the levels (cell counts) of circulating 2B4+ CD4 T cells are decreased relative to a control or reference.

In another aspect, the present disclosure provides a method of treating a cancer in a human subject in need thereof. The method includes (a) determining levels (cell counts) of circulating T helper cells in a biological sample from the subject; and (b) administering a CD40 agonist in combination with a chemotherapeutic agent to the subject if the levels (cell counts) of circulating T helper cells are increased relative to a control or reference.

The present disclosure also provides a method of treating a cancer in a human subject in need thereof. The method includes (a) determining an E2F gene signature in a biological sample from the subject and calculating an E2F signature score; and (b) administering a CD40 agonist in combination with a chemotherapeutic agent to the subject if the E2F gene signature score is decreased relative to a control or reference.

In one aspect, the method comprises calculating the E2F gene signature score by averaging log normalized expression values for each gene in an E2F gene set. In one aspect, the E2F gene set comprises one or more genes selected from the group consisting of ABCE1, ACP1, AIMP2, AP3S1, APEX1, BUB3, C1QBP, CAD, CANX, CANX, CBX3, CCNA2, CCT2, CCT3, CCT4, CCT5, CCT7, CDC20, CDC45, CDK2, CDK4, CLNS1A, CNBP, COPS5, COX5A, CSTF2, CTPS1, CUL1, CYC1, DDX18, DDX21, DEK, DHX15, DUT, EEF1B2, EIF1AX, EIF2S1, EIF2S2, EIF3B, EIF3D, EIF3J, EIF4A1, EIF4E, EIF4G2, EIF4H, EPRS1, ERH, ETF1, EXOSC7, FAM120A, FBL, G3BP1, GLO1, GNL3, GOT2, GSPT1, H2AZ1, HDAC2, HDDC2, HDGF, HNRNPA1, HNRNPA2B1, HNRNPA3, HNRNPC, HNRNPD, HNRNPR, HNRNPU, HPRT1, HSP90AB1, HSPD1, HSPE1, IARS1, IFRD1, ILF2, IMPDH2, KARS1, KPNA2, KPNB1, LDHA, LSM2, LSM2, LSM7, MAD2L1 MCM2, MCM4, MCM5, MCM6, MCM7, MRPL23, MRPL23, MRPL9, MRPS18B, MYC, NAP1L1, NCBP1, NCBP2, NDUFAB1, NHP2, NME1, NOLC1, NOP16, NOP56, NPM1, ODC1, ORC2, PA2G4, PABPC1, PABPC4, PCBP1, PCNA, PGK1, PHB, PHB2, POLD2, POLE3, PPIA, PPM1G, PRDX3, PRDX4, PRPF31, PRPS2, PSMA1, PSMA2, PSMA4, PSMA6, PSMA7, PSMB2, PSMB3, PSMC4, PSMC4, PSMC6, PSMD1, PSMD14, PSMD3, PSMD7, PSMD8, PTGES3, PWP1, RACK1, RAD23B, RAN, RANBP1, RFC4, RNPS1, RPL14, RPL18, RPL22, RPL34, RPL6, RPLP0, RPS10, RPS2, RPS3, RPS5 RPS6, RRM1, RRP9, RSL1D1, RUVBL2, SERBP1, SET, SF3A1, SF3B3, SLC25A3, SMARCC1, SNRPA, SNRPA1, SNRPB2, SNRPD1, SNRPD2, SNRPD3, SNRPG, SRM, SRPK1, SRSF1, SRSF2, SRSF3, SRSF7, SSB, SSBP1, SSBP1, STARD7, SYNCRIP, TARDBP, TCP1, TFDP1, TOMM70, TRA2B, TRIM28, TUFM, TXNL4A, TYMS, U2AF1, UBA2, UBE2E1, UBE2L3, USP1, VBP1, VDAC1, VDAC3, XPO1, XPOT, XRCC6, YWHAE, YWHAE, and YWHAQ.

In a further aspect, the present disclosure provides a method of treating a cancer in a human subject in need thereof. The method includes (a) determining an IFN-γ gene signature in a biological sample from the subject and calculating an IFN-γ gene signature score; and (b) administering a CD40 agonist in combination with a chemotherapeutic agent to the subject if the IFN-γ gene signature score is increased relative to a control or reference.

In one aspect, the method further comprises calculating the IFN-γ gene signature score by averaging log normalized expression values for each gene in an IFN-γ gene set. In one aspect, the IFN-γ gene set comprises one or more genes selected from the group consisting of CD8A, CD274, LAG3, and STAT1. In one aspect, the biological sample is a liquid biopsy optionally a blood or serum sample, a surgical sample, or other biopsy sample obtained from the subject. In one aspect, the method further includes performing step (a) prior to initiating treatment with the CD40 agonist. In one aspect, the cancer is selected from pancreatic cancer, an endometrial cancer, a non-small cell lung cancer (NSCLC), a renal cell carcinoma, a urothelial cancer, a head and neck cancer, a melanoma, a bladder cancer, a hepatocellular carcinoma, a breast cancer, an ovarian cancer, a gastric cancer, a colorectal cancer, a glioblastoma, a biliary tract cancer, a glioma, Merkel cell carcinoma, Hodgkin lymphoma, non-Hodgkin lymphoma, a cervical cancer, an advanced or refractory solid tumor, a small cell lung cancer, a non-squamous non-small cell lung cancer, desmoplastic melanoma, a pediatric advanced solid tumor or lymphoma, a mesothelin-positive pleural mesothelioma, an esophageal cancer, an anal cancer, a salivary cancer, a prostate cancer, a carcinoid tumor, a primitive neuroectodermal tumor (pNET), and a thyroid cancer. In one aspect, the cancer is a pancreatic cancer, optionally a pancreatic ductal adenocarcinoma (PDAC). In one aspect, the CD40 agonist is an antibody, or an antigen-binding fragment thereof, which specifically binds to and agonizes human CD40. In one aspect, the antibody, or antigen-binding fragment thereof, is selected from the group consisting of sotigalimab, selicrelumab, ChiLob7/4. ADC-1013, SEA-CD40, CP-870,893, dacetuzumab, and CDX-1140. In one aspect, the chemotherapeutic agent is selected from the group consisting of gemcitabine, nab-paclitaxel, folfirionx, nitrogen mustard/oxazaphosphorine, nitrosourea, triazene, and alkyl sulfonates, anthracycline antibiotics such as doxorubicin and daunorubicin, taxanes such as Taxol and docetaxel, vinca alkaloids such as vincristine and vinblastine, 5-fluorouracil (5-FU), leucovorin, Irinotecan, idarubicin, mitomycin C, oxaliplatin, raltitrexed, pemetrexed, tamoxifen, cisplatin, carboplatin, methotrexate, actinomycin D, mitoxantrone, brenoxane, mitramycin, methotrexate, paclitaxel, 2-methoxyestradiol, purinomastert, batimastat, BAY 12-9656, carboxamidotriazole, CC-1088, dextromethorphan acetic acid, dimethylxanthenone acetic acid, Endostatin, IM-862, marimastat, penicillamine, PTK787/ZK 222584, RPI 4610, squalamine lactate, SU5416, thalidomide, combretastatin, tamoxifen, COL-3, neobasstat, BMS-275291, SU6668, anti-VEGF antibody, Med-522 (Vitaxin II), CAI, interleukin 12, IM862, amiloride, Angiostatin, angiostatin Kl-3, angiostatin K1-5, captopril, DL-α-difluoromethylomithine, DL-α-difluoromethylomithine HCl, endostatin, fumagillin, herbimycin A, 4-hydroxyphenylretinamide, Juglone, laminin, laminin hexapeptide, laminin pentapeptide, labendustin A, medroxyprogesterone, minocycline, placental ribonuclease Inhibitors, suramin, thrombospondin, antibodies targeting pro-angiogenic factors, topoisomerase inhibitors, microtubule inhibitors, low-molecular-weight tyrosine kinase inhibitors of pro-angiogenic growth factors agents, GTPase inhibitors, histone deacetylase inhibitors, AKT kinase or ATPase inhibitors, Win (Wnt) signal inhibitors, E2F transcription factor inhibitors, mTOR inhibitors Agents, α, β, and γ interferons, IL-12, matrix metalloproteinase inhibitors, Z06474, SU1248, vitaxin, POGFR inhibitors, NM3 and 2-ME2, and sirengitide. In one aspect, the chemotherapeutic agent is a combination of gemcitabine and nab-paclitaxel.

Each of the aspects and embodiments described herein are capable of being used together, unless excluded either explicitly or clearly from the context of the embodiment or aspect.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows treatment cohorts and analysis populations of the Phase 2 trial described in Examples 1-6.

FIG. 2 shows the percent changes in the sum of target lesions of efficacy study in the Phase 2 trial described in Examples 1-6.

FIG. 3 shows overall survival (OS) rate of the cohorts in the Phase 2 trial described in Examples 1-6.

FIG. 4A shows an immune profiling of peripheral blood mononuclear cells (PBMCs), showing an increase in activated effector memory (EM) T cells (Ki67⁺CD8⁺) in all three cohorts, with cohort A1 (nivolumab+chemotherapy) showing the most pronounced effect.

FIG. 4B shows immune profiling of peripheral blood mononuclear cells (PBMCs), showing an increase in activated myeloid dendritic cells (CD86⁺mDC) in cohort B2 and C2. Cohort A1 predominantly showed a decrease.

FIG. 5A shows tumor multiplex IHC analyses of all three cohorts, showing a decrease in the percentage of tumor cells expressing PD-L1 in cohorts A1 and C2, while cohort B2 showed mixed changes in PD-L1 expression.

FIG. 5B shows tumor multiplex IHC analyses of all three cohorts, showing an increase in tumoral CD80+M1 macrophages in cohort B2, while cohorts A1 and C2 showed a decrease.

FIG. 6 shows microbiome profiling of stool samples of all three cohorts, showing cohort A1 had increased bacteroidia and decreased clostridia, while cohort B2 showed the opposite. FIG. 7A shows patient survival stratified by baseline immune profiling of CXCR5⁺ effector memory CD8⁺ T cells of all three cohorts.

FIG. 7B shows patient survival stratified by baseline immune profiling of CD244⁺ effector memory CD4⁺ T cells of all three cohorts.

FIG. 8A shows patient survival stratified by baseline TNFα tumor gene expression profiling from RNA Seq analyses of all three cohorts.

FIG. 8B shows patient survival stratified by baseline MYC tumor gene expression profiling from RNA Seq analyses of all three cohorts.

FIG. 9 shows CyTOF gating strategy. Gating strategy used to define immune cell populations by CyTOF analysis is shown. Representative flow plots are shown.

FIG. 10 shows T cell phenotyping gating strategy. Gating strategy used to define T cell populations by flow cytometry analysis is shown. Representative flow plots are shown.

FIG. 11 shows single marker controls for mIF. Equivalency of single-marker optimized antibody immunohistochemistry (IHC) developed with 3,3′-diaminobendidine (DAB) on human tonsil tissue (top rows) with corresponding multiplexed immunofluorescence (mIF) on tonsil (bottom rows). The immunofluorescent images represent individual marker position within the 7-color assay performed.

FIGS. 12A-12B show PRINCE Study Design and CONSORT Diagram. FIG. 12A shows that PRINCE was a seamless phase 1b/2 study, with the phase 2 portion randomizing patients to treatment with nivo/chemo, sotiga/chemo, or sotiga/nivo/chemo. FIG. 12B is a CONSORT diagram of the phase 2 portion of the study. Patients enrolled in Cohorts B2 and C2 during phase 1b were included in safety and/or efficacy analyses of the phase 2 portion.

FIGS. 13A-13B show activated T cells frequencies increase with nivo/chemo treatment. FIG. 13A shows the frequencies of circulating CD38+CD8 (left panel) and CD4 (right panel) non-naïve T cells, as a fraction of total non-naive CD8 or CD4 T cells respectively, in patients from each cohort pretreatment and on-treatment. Shown as fold change relative to C1D1 (pretreatment) and plotted on a pseudo-log scale. Dark line indicates median values and error bars represent 95% CI. P-values represent probability of non-zero slope for line of best fit along the full series. FIG. 13B shows the frequencies of circulating CD39+ non-naïve CD8 (left panel) and CD4 (right panel) T cells, as a fraction of total non-naïve CD8 or CD4 T cells respectively, in patients from each cohort pretreatment and on-treatment. Shown as fold change relative to C1D1 (pretreatment) and plotted on a pseudo-log scale. Dark line indicates median values and error bars represent 95% CI. P-values represent probability of non-zero slope for line of best fit along the full series. **See Table 7 for number of samples in applicable analyses.

FIGS. 14A-14E show biomarker signatures in blood and tumor reveal specific immune mechanisms of activation in response to nivo/chemo and sotiga/chemo treatment in patients with mPDAC. FIG. 14A shows the frequencies of circulating Ki-67+ non-naïve CD8 (left panel) and CD4 (right panel) T cells, as a fraction of total non-naïve CD8 or CD4 T cells respectively, in patients from each cohort pretreatment and on-treatment. Shown as fold change relative to C1D1 (pretreatment) and plotted on a pseudo-log scale. Dark line indicates median values and error bars represent 95% CI. P-values represent probability of non-zero slope for line of best fit along the full series. FIG. 14B shows representative flow plots using PBMC samples over time from a patient in the nivo/chemo treatment arm showing an increase in Ki-67+ non-naïve CD8 (top panel) and CD4 (bottom panel) T cells. FIG. 14C shows volcano plots showing circulating proteins significantly up or downregulated from C1D1 (pretreatment) to C1D15 (left) or C2D1 (right) for each of the three cohorts. Dotted lines indicate FDR value of 0.05 and fold change of 2 in Log2 protein expression. Proteins of interest related to immune mechanisms are highlighted.

FIG. 14D and FIG. 14E show frequencies of PD-L1+ tumor cells (FIG. 14D, left panel) and intratumoral iNOS CD80 (FIG. 14E, left panel) macrophages from multiplex IHC of on-treatment biopsies (C2D1 when feasible, see methods for details), shown as fold change relative to pretreatment biopsy, for each cohort. P-values represent Wilcoxon signed-rank test between the pretreatment and (not-normalized) on-treatment cell proportions. Representative images are shown from patients in the nivo/chemo cohort (PD-L1+ tumor cells) (FIG. 14D, right panel) and sotiga/chemo arm (iNOS CD80 macrophages) (FIG. 14E, right panel). **See Table 7 for number of samples in applicable analyses.

FIGS. 15A-15F show activated T cells and type-1 immune responses increase with nivo/chemo treatment, whereas proteins critical for helper responses & innate immune responses increase with sotiga/chemo treatment in patients with mPDAC. FIG. 15A shows frequencies of circulating HLA-DR+ non-naïve CD4 (left panel) or CD8 (right panel) T cells in patients from each cohort pretreatment and on-treatment. FIG. 15B shows representative flow plots from PBMC samples over time from a patient in the nivo/chemo treatment arm depicting increasing HLA-DR+ non-naïve CD8 (top) and CD4 (bottom) T cells. FIGS. 15C-15F show circulating IFNγ (FIG. 15C), PD-1 (FIG. 15D), CXCL9 (FIG. 15E) and CXCL10 (FIG. 15F) on-treatment fold change from pretreatment (C1D1) of Log 2 expression values from each cohort, plotted on a pseudo-log scale. Timeseries plots in a-f show median values in thick lines and individual patient values in thin lines, and error bars represent 95% confidence intervals. P-values on timeseries plots represent p-value of non-zero slope for line of best fit along the full series. FIGS. 15G-15H show DIABLO Circos plot (FIG. 15G) and correlation matrix (FIG. 15H) showing factors from CyTOF, ×50 flow cytometry, Olink, protein mass spectrometry significantly associated with treatment and correlations among these factors. In circus plot (FIG. 15G), lines outside the circle indicate magnitude and direction of treatment association. Lines inside the plot indicate positive and negative correlations between biomarker factors. In correlation plot (FIG. 15H), color of text indicates the treatment association of the biomarker. For all cell populations shown, frequencies are out of parent population. **See Table 7 for number of samples in applicable analyses.

FIGS. 16A-H show a non-immunosuppressive tumor microenvironment and activated circulating CD8 T cells before treatment are associated with survival in mPDAC patients treated with nivo/chemo. FIG. 16A is a heatmap of gene expression signatures that associate significantly with survival outcomes in response to nivo/chemo treatment between higher (above median) and lower (below median) values in pretreatment tumor samples. Individual patients are shown in columns and annotated by survival status at 1 year to illustrate association. FIG. 16B shows Kaplan-Meier (KM) curves for overall survival stratified by TNFα via NFκB hallmark pathway signature above and below the median signature value across all patients in all cohorts. FIG. 16C is a KM curve for overall survival stratified by percentage of iNOS+ intratumoral macrophages out of total macrophages from mIF of pretreatment biopsies, above and below the median percentage across all patients in all cohorts (FIG. 16C, top panel). Representative pretreatment tumor mIF images from two patients (FIG. 16C, bottom panel). FIG. 16D shows the percentage of tumor cells in pretreatment biopsies expressing PD-L1 by mIF, stratified by overall survival status at 1 year. P value is a Wilcoxon signed-rank test. FIG. 16E shows a correlation matrix of immune percentages and gene expression signatures in pretreatment tumor biopsies, with labels color coded by association with survival outcome. FIG. 16F is a heatmap of median fluorescence intensity of proteins on CD38+ Effector Memory CD8 T cell population from pretreatment PBMC samples across patients in the nivo/chemo cohort. FIG. 16G shows KM curves for overall survival stratified by frequencies of circulating CD38+ CD8 Effector Memory T cells out of total CD8 T cells, at baseline above and below the median frequency. Frequencies of CD38 CD8 T cells out of total CD8 T cells in pretreatment and on-treatment PBMC samples (C1D15, C2D1, C4D1), segregated by patient survival status at 1 year. P-values represent Wilcoxon signed-rank test between timepoints to show increases in cell proportions on-treatment. FIG. 16H shows multi-omic dimensionality reduction of circulating factors and tumor data using Independent Component Analysis, with each dot representing a single patient colored by survival status at one year and with position determined by reduced dimensionality across all tumor and circulating biomarkers. For all cell populations shown, frequencies are out of parent population. On all KM curves, P-values are from a log-rank test between groups, and shaded regions illustrate 95% CI. **See Table 7 for number of samples in applicable analyses.

FIG. 17 shows PD-L1 expression on tumor cells prior to treatment trends with longer survival in in mPDAC patients treated with nivo/chemo. Percentage of tumor cells in pretreatment biopsies expressing PD-L1 by multiplex IHC, stratified by overall survival status at 1 year. P-value is a Wilcoxon signed-rank test.

FIGS. 18A-18F show antigen-experienced Non-Naïve T cells and follicular helper T cells in the periphery are associated with survival in mPDAC patients treated with nivo/chemo.

FIG. 18A shows KM curves for overall survival stratified by frequencies of circulating PD-1+CD39+ Effector Memory 1 CD4 T cells above and below the median across all patients in all cohorts. FIG. 18B is a heatmap of median fluorescence intensity of proteins present on pretreatment PD-1+CD39+ Effector Memory 1 CD4 T cells across all patients in the nivo/chemo cohort. FIG. 18C shows frequencies of PD-1+CD39+ Effector Memory 1 CD4 T cells pretreatment and on-treatment (C1D15, C2D1, C4D1). FIG. 18D shows KM curves for overall survival stratified by frequencies of circulating T Follicular Helper (CXCR5+PD-1+CD4+) T cells above and below the median across all patients in all cohorts. FIG. 18E is a heatmap of median fluorescence intensity of proteins present on pretreatment T Follicular Helper T cells across all patients in the nivo/chemo cohort. FIG. 18F shows frequencies of T Follicular Helper T cells pretreatment and on-treatment (C1D15, C2D1, C4D1). For all cell populations shown, frequencies are out of parent population. Time series plots show box plots with median and quartiles in thick lines and individual patient values in thin lines, colored by survival status at 1 year. P-values for timeseries represent Wilcoxon signed-rank tests between survival groups at each timepoint. On KM curves, P-values are from a log-rank test between groups, and shaded regions illustrate 95% CI.

FIGS. 19A-19C show antigen-experienced Non-Naïve Central Memory T cells and follicular helper T cells in the periphery are associated with survival in mPDAC patients treated with nivo/chemo. FIG. 19A shows KM curves for overall survival stratified by frequencies of circulating PD-1+CD39+ Central Memory CD4 T cells above and below the median across all patients in all cohorts. FIG. 19B is a heatmap of median fluorescence intensity of proteins present on pretreatment PD-1+CD39+ Central Memory CD4 T cells across all patients in the nivo/chemo cohort. FIG. 19C shows frequencies of PD-1+CD39+ Central Memory CD4 T cells pretreatment and on-treatment (C1D15, C2D1, C4D1). **See Table 7 for number of samples in applicable analyses.

FIGS. 20A-20G show helper signatures and proliferating CD4 T cells in the tumor associate with survival in patients receiving sotiga/chemo treatment FIG. 20A is a heatmap of gene expression signatures that are significantly associated with survival in response to sotiga/chemo treatment between higher (above median) and lower (below median) values in pretreatment tumor biopsies. Individual patients are shown in columns, with a label corresponding to one-year overall survival status. FIGS. 20B-20D show KM curves for overall survival stratified by Th1 (FIG. 20B), IFNγ (FIG. 20C), and E2F (FIG. 20D) gene expression signatures above and below the median. FIG. 20E shows a KM curve for overall survival stratified by Ki-67− Foxp3− CD4 T cells from mIF on pretreatment tumor samples above and below the median (FIG. 20E, top panel) and representative images from tumor samples high and low in Ki-67− Foxp3− CD4 T cells (FIG. 20E, bottom panel) with associated patient survival values. FIG. 20F shows a correlation matrix of immune infiltrate and gene expression signatures in pretreatment tumor biopsies, colored by association with overall survival outcome. FIG. 20G is a DIABLO Circos plot showing factors from RNAseq (gx) and Vectra imaging significantly associated with survival status at 1 year, and correlations among these factors. Lines outside the circle indicate magnitude and direction of treatment association. Lines inside the plot indicate positive and negative correlations between biomarker factors. On all KM curves, P-values are from a log-rank test between groups, and shaded regions illustrate 95% CI. **See Table 7 for number of samples in applicable analyses.

FIGS. 21A-21B shows overall survival and tumor response. FIG. 21A shows overall survival of patients in the efficacy population. FIG. 21B shows the maximum percentage change from baseline in the sum of the diameters of the target lesions for each patient with post-baseline tumor assessments. Four patients in the nivo/chemo arm, 1 in the sotiga/chemo arm, and 3 in the sotiga/nivo/chemo arm did not have any post-baseline tumor assessments. Confirmed complete response (CR) or partial response (PR) is defined as two consecutive tumor assessments with an overall response of complete/partial response.

FIGS. 22A-22L show cross-presenting, activated APCs and type-1 helper T cells in circulation associate with survival in patients receiving sotiga/chemo treatment. FIG. 22A shows force-directed graph visualization of unsupervised clustering of cells from CyTOF across all patients and timepoints, illustrating a specific population of dendritic cells associating with survival and followed up on with gating analysis in further panels. FIG. 22B shows a timeseries (top) and KM curve (bottom) for overall survival stratified by C1D1 frequency of circulating CD1c+ cross presenting DCs (CD141+), above and below the median at C1D1. FIG. 22C shows a timeseries (top) and KM curve (bottom) for overall survival stratified by C1D15 frequency of circulating cross presenting DCs (CD141+), above and below the median at C1D15.FIG. 22D shows a timeseries (top) and KM curve (bottom) for overall survival stratified by C1D15 frequency of circulating CD1c− cross presenting DCs (CD141+), above and below the median at C1D15. FIG. 22E shows a timeseries (top) and KM curve (bottom) for overall survival stratified by C2D1 frequency of circulating conventional DCs, above and below the median at C2D1. FIG. 22 F shows KM curves for overall survival stratified by frequency of pretreatment circulating PD-1+ Tbet+ non-naïve CD4 T cells. FIG. 22G is a heatmap of pretreatment mean fluorescence intensity of proteins present on PD-1+ Tbet+ non-naïve CD4 T cells across all patients. FIG. 22H shows the frequency of PD-1+ Tbet+ non-naïve CD4 T cells pretreatment and on-treatment (C1D1, C1D15, C2D1, and C4D1), colored by survival status at 1 year. FIG. 22I shows KM curves for overall survival stratified by frequency of pretreatment circulating Tbet+ Eomes+ non-naïve CD4 T cells. FIG. 22J is a heatmap of pretreatment mean fluorescence intensity of proteins present on Thet+ Eomes+ non-naïve CD4 T cells across all patients. FIG. 22K shows the frequency of Thet+ Eomes+ non-naïve CD4 T cells pretreatment and on-treatment (C1D1, C1D15, C2D1, and C4D1), colored by survival status at 1 year. FIG. 22L shows multi-omic dimensionality reduction of circulating factors and tumor data using Independent Component Analysis, with each dot representing a single patient colored by survival status at one year and with position determined by reduced dimensionality across all tumor and circulating biomarkers. For dendritic cell populations, frequencies are out of total leukocytes. For T cell populations, frequencies are out of parent. Time series plots show box plots with median and quartiles in thick lines and individual patient values in thin lines, colored by survival status at 1 year. P-values for timeseries represent Wilcoxon signed-rank tests between survival groups at each timepoint. On KM curves, P-values are from a log-rank test between groups, and shaded regions illustrate 95% CI. **See Supplementary Table 19 for number of samples in applicable analyses.

FIGS. 23A-23B show soluble molecules associated with Dendritic Cell Maturation are associated with survival on-treatment (C1D15) in mPDAC patients treated with sotiga/chemo. FIG. 23A shows Kaplan-Meier (KM) curves for overall survival stratified by soluble CD83 protein expression above and below the median signature value across all patients in all cohorts at C1D15. FIG. 23B shows Kaplan-Meier (KM) curves for overall survival stratified by soluble ICOSL protein expression above and below the median signature value across all patients in all cohorts at C1D15. **See Table 7 for number of samples in applicable analyses.

FIGS. 24A-24E show higher frequencies of specific B cell populations and lower concentrations of 2B4+ T cells are associated with survival in patients treated with sotiga/chemo. FIG. 24A shows force-directed graph visualization of unsupervised clustering of cells from CyTOF across all patients and timepoints, illustrating a specific population of B cells associating with survival and followed up on with gating analysis in further panels. FIG. 24B shows KM curves for overall survival stratified by frequencies of pretreatment circulating HLA-DR+ CCR7+ B cells out of total leukocytes, above and below the median frequency. FIG. 24C shows KM curves for overall survival stratified by frequency of pretreatment circulating 2B4+ non-naïve CD4 T cells out of total non-naïve CD4 T cells. FIG. 24D shows a heatmap of pretreatment mean fluorescence intensity of proteins present on 2B4+ non-naïve CD4 T cells across all patients. FIG. 24E shows the frequency of 2B4+ Non-Naïve CD4 T cells pretreatment and on-treatment (C1D1, C1D15, C2D1, and C4D1). Plot shows box plots with median and quartiles in thick lines and individual patient values in thin lines, colored by survival status at 1 year. P-values represent Wilcoxon signed-rank tests between timepoints, illustrating increases on-treatment. On all KM curves, P-values are from a log-rank test between groups, and shaded regions illustrate 95% CI. **See Table 7 for number of samples in applicable analyses.

FIG. 25 shows biomarkers of survival following nivo/chemo and sotiga/chemo, and their overlap. Venn diagrams of broad categories of circulating biomarkers (Top). Left circle shows biomarkers of survival following sotiga/chemo, right circle shows biomarkers of survival following nivo/chemo, and center shows overlapping biomarkers which are associated with survival in both treatment groups. Color indicates direction of association, with blue for higher values associating with longer survival, and red for higher values associating with shorter survival. The same structure is shown for tumor biomarkers (Bottom).

FIG. 26A-26E shows lower frequencies of circulating CD38+ Non-Naïve T cells are associated with longer survival in patients treated with sotiga/nivo/chemo. a, b, KM curves for overall survival stratified by frequencies of circulating CD38+ non-naïve CD4 (FIG. 26A) and CD8 (FIG. 26B) T cells at baseline, above and below the median frequency value. c, Heatmaps of median fluorescent intensity of pretreatment proteins present on CD38+ non-naïve CD4 and CD8 T cells across all patients. FIGS. 26D-26E show frequencies of CD38+ non-naïve CD4 (FIG. 26D) and CD8 (FIG. 26E) T cells are shown pretreatment and on-treatment. For all cell populations shown, frequencies are out of parent. Time series plots show box plots with median and quartiles in thick lines and individual patient values in thin lines, colored by survival status at 1 year. P-values for timeseries represent Wilcoxon signed-rank tests between timepoints. On KM curves, P-values are from a log-rank test between groups, and shaded regions illustrate 95% CI. **See Table 7 for number of samples in applicable analyses.

FIGS. 27A-27D show survival in response to combinational therapy of sotiga, nivo and chemo may be affected by regulatory B cells in circulation. FIG. 27A shows frequencies of circulating CCR7+CD11b+CD27− B cells in patients from each treatment arm pretreatment and on-treatment (C1D15, C2D1, C4D1), shown as fold change relative to C1D1 and plotted on a pseudo-log scale. FIG. 27B shows KM curves for overall survival stratified by CCR7+CD11b+CD27− B cells above and below the median frequency value. FIG. 27C is a heatmap of median fluorescent intensity of different proteins present on CCR7+CD11b+CD27− B cells on-treatment (C1D15) across all patients. FIG. 27D shows frequencies of CCR7+CD11b+CD27− B cells pretreatment and on-treatment (C1D15, C2D1, C4D1), stratified by overall survival status at 1 year, for each treatment arm. For all cell populations shown, frequencies are out of total leukocytes. Time series plots show median value in thick lines, individual patient value in thin lines, and error bars are 95% confidence intervals. P-values for timeseries represent Wilcoxon signed-rank tests between survival groups at each timepoint. On KM curves, P-values are from a log-rank test between groups, and shaded regions illustrate 95% CI. **See Table 7 for number of samples in applicable analyses.

The following description and examples illustrate embodiments of the present disclosure in detail.

It is to be understood that the present disclosure is not limited to the particular embodiments described herein and as such can vary. Those of skill in the art will recognize that there are variations and modifications of the present disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment.

### DETAILED DESCRIPTION

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

Compositions or methods “comprising” or “including,” or any grammatical variant thereof, one or more recited elements can include other elements not specifically recited. For example, a composition that includes antibody can contain the antibody alone or in combination with other ingredients.

Certain ranges are presented herein with numerical values being preceded by the term “about.” The term “about” is used herein has its original meaning of approximately and is to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, if the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The term “treatment” or any grammatical variant thereof of a cancer as used herein means to administer a combination therapy of a CD40 agonist, such as an anti-CD40 antibody (e.g., sotigalimab) and one or more chemotherapy drugs to a subject having the cancer, or diagnosed with the cancer, to achieve at least one positive therapeutic effect, such as for example, reduced number of cancer cells, reduced tumor size, reduced rate of cancer cell infiltration into peripheral organs, or reduced rate of tumor metastasis or tumor growth. Positive therapeutic effects in cancer can be measured in a number of ways (See, W. A. Weber, J. Nucl. Med. 50: 1S-10S (2009)). The treatment regimen for the disclosed combination that is effective to treat a cancer patient can vary according to factors such as the disease state, age, and weight of the patient, and the ability of the therapy to elicit an anti-cancer response in the subject. The treatment methods, medicaments, and disclosed uses may not be effective in achieving a positive therapeutic effect in every subject, they should do so in a statistically significant number of subjects as determined by any statistical test known in the art.

The term “antibody” includes intact antibodies and binding fragments thereof that specifically bind to a single antigen or that specifically bind to multiple antigens (e.g., multispecific antibodies such as a bispecific antibody, a trispecific antibody, etc.). Thus, any reference to an antibody should be understood to refer to the antibody in intact form or a binding fragment unless the context requires otherwise.

The term “binding fragment,” which can be used interchangeably with “antigen-binding fragment,” refers herein to an antibody fragment formed from a portion of an antibody comprising one or more CDRs, or any other antibody fragment that specifically binds to an antigen but does not comprise an intact native antibody structure. Examples of antigen-binding fragment include, without limitation, a diabody, a Fab, a Fab′, a F(ab′)2, a F(ab)c, an Fv fragment, a disulfide stabilized Fv fragment (dsFv), a (dsFv)₂, a bispecific dsFv (dsFv-dsFv′), a disulfide stabilized diabody (ds diabody), a triabody, a tetrabody, a single-chain antibody molecule (scFv), an scFv dimer, a multispecific antibody, a camelized single domain antibody, a nanobody, a minibody, a domain antibody, a bivalent domain antibody, a IgNAR, a V-NAR, and a hcIgG. Binding fragments can be produced by recombinant DNA techniques, or by enzymatic or chemical separation of intact immunoglobulins.

“Fab” with regard to an antibody refers to that portion of the antibody consisting of a single light chain (both variable and constant regions) bound to the variable region and first constant region of a single heavy chain by a disulfide bond.

“Fab′” refers to a Fab fragment that includes a portion of the hinge region.

“F(ab′)₂” refers to a dimer of Fab′.

“Fc” with regard to an antibody refers to that portion of the antibody consisting of the second and third constant regions of a first heavy chain bound to the second and third constant regions of a second heavy chain via disulfide bonding. The Fc portion of the antibody is responsible for various effector functions such as ADCC, and CDC, but does not function in antigen binding.

“Fv” with regard to an antibody refers to the smallest fragment of the antibody to bear the complete antigen binding site. An Fv fragment consists of the variable region of a single light chain bound to the variable region of a single heavy chain.

“Single-chain Fv antibody” or “scFv” refers to an engineered antibody consisting of a light chain variable region and a heavy chain variable region connected to one another directly or via a peptide linker sequence (Huston J. S. et al., Proc Natl Acad Sci USA, 85:5879 (1988)).

“Single-chain Fv-Fc antibody” or “scFv-Fc” refers to an engineered antibody consisting of a scFv connected to the Fc region of an antibody.

“Camelized single domain antibody,” “heavy chain antibody,” or “HCAb” refers to an antibody that contains two V_{H} domains and no light chains (Riechmann L. and Muyldermans S., J Immunol Methods. December 10; 231(1-2): 25-38 (1999); Muyldermans S., J Biotechnol. June; 74(4):277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V. K. et al. “Heavy-chain antibodies in Camelidae; a case of evolutionary innovation,” Immunogenetics. April; 54(1):39-47 (2002); Nguyen V. K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)).

“Nanobody” refers to an antibody fragment that consists of a VHH domain from a heavy chain antibody and two constant domains, CH2 and CH3.

“Diabody” refers to a small antibody fragment with two antigen-binding sites, wherein the fragments comprise a V_{H} domain connected to a V_{L} domain in the same polypeptide chain (V_{H}-V_{L} or V_{L}-V_{H}) (see, e.g., Holliger P. et al., Proc Natl Acad Sci USA. July 15; 90(14):6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites can target the same or different antigens (or epitopes).

“Domain antibody” refers to an antibody fragment containing only the variable region of a heavy chain or the variable region of a light chain. In certain instances, two or more V_{H} domains are covalently joined with a peptide linker to create a bivalent or multivalent domain antibody. The two V_{H} domains of a bivalent domain antibody can target the same or different antigens.

In certain embodiments, a “(dsFv)₂” comprises three peptide chains: two V_{H} moieties linked by a peptide linker and bound by disulfide bridges to two V_{L} moieties.

In certain embodiments, a “bispecific ds diabody” comprises V_{H1}-V_{L2} (linked by a peptide linker) bound to V_{L1}-V_{H2} (also linked by a peptide linker) via a disulfide bridge between V_{H1} and V_{L1}.

In certain embodiments, a “bispecific dsFv” or dsFv-dsFv′” comprises three peptide chains: a V_{H1}-V_{H2} moiety wherein the heavy chains are linked by a peptide linker (e.g., a long flexible linker) and bound to V_{L1} and V_{L2} moieties, respectively, via disulfide bridges, wherein each disulfide paired heavy and light chain has a different antigen specificity.

In certain embodiments, an “scFv dimer” is a bivalent diabody or bivalent ScFv (BsFv) comprising V_{H}-V_{L} (linked by a peptide linker) dimerized with another V_{H}-V_{L} moiety such that V_{H}'s of one moiety coordinate with the V_{L}'s of the other moiety and form two binding sites which can target the same antigens (or epitopes) or different antigens (or epitopes). In other embodiments, an “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

The term “biological sample” or “sample” refers to any solid or liquid sample isolated from an individual or a subject. For example, it can refer to any solid (e.g., tissue sample) or liquid sample (e.g., blood) isolated from an animal (e.g., human), such as, without limitations, a biopsy material (e.g., solid tissue sample), or blood (e.g., whole blood). Such sample can be, for example, fresh, fixed (e.g., formalin-, alcohol- or acetone-fixed), paraffin-embedded or frozen prior to an analysis. In an embodiment, the biological sample is obtained from a tumor (e.g., a pancreatic cancer). A “test biological sample” is the biological sample that has been the subject of analysis, monitoring, or observation. A “reference biological sample,” containing the same type of biological sample (e.g., the same type of tissues or cells), is a control for the test biological sample.

The term “gene signature” refers to a hallmark gene signature publicly accessible through the Molecular Signatures Database (MSigDB) (V7.4) for gene set enrichment analysis (GSEA). Hallmark gene sets are coherently expressed signatures derived by aggregating many MSigDB gene sets to represent well-defined biological states or processes. For example, MYC hallmark gene set include genes belonging to tumor suppressors, oncogenes, translocated cancer genes, protein kinases, cell differentiation markers, homeodomain proteins, Transcription factors, and cytokine and growth factors.

As used herein, an “individual” or a “subject” includes animals, such as human (e.g., human individuals) and non-human animals. In some embodiments, an “individual” or “subject” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub combination was individually and explicitly disclosed herein.

### II. Methods of the Identifying a Subset of Cancer Patients

Provided herein are, inter alia, methods of identifying a subset of cancer patients for a treatment with a CD40 agonist, such as an anti-CD40 antibody (e.g., sotigalimab). In some embodiments, the treatment is combined with one or more chemotherapy drugs (e.g., gemcitabine and nab-paclitaxel). Provided herein are methods for treating cancer as well as methods of aiding cancer treatment in the identified subset of cancer patients. Also provided herein is a system and/or kit for identifying the subset of cancer patients amenable to the treatment.

To assess whether a subject will respond effectively to a combination therapy comprising a CD40 agonist (e.g., an anti-CD40 antibody such as sotigalimab or CD40 ligand-fusion protein) and chemotherapy (e.g., gemcitabine and nab-paclitaxel) or to evaluate continued treatment with this combination therapy, the following methods can be employed.

The term a “CD40 agonist” is an agent that specifically binds to CD40 to activate CD40 similar to the binding of CD40 ligand. A CD40 agonist can include a compound that binds to CD40 for receptor activation. A CD40 agonist can also be a compound that mimics the CD40 ligand that binds and activates CD40. The CD40 agonist can be an antibody, e.g. a monoclonal antibody or antigen-binding fragment thereof to CD40. When a specific biologic name is referring to herein, it also can include its biosimilar as well as the reference product biologic. Exemplary antibodies or antigen-binding fragments thereof include, without limitation, sotigalimab, selicrelumab, ChiLob7/4, ADC-1013, SEA-CD40, CP-870,893, dacetuzumab, and CDX-1140.

A test biological sample (e.g., a bulk tumor tissue and/or blood) and one or more reference or control biological samples can be obtained from a test subject having a particular type of cancer and one or more reference subjects having the same type of cancer as the test subject both prior to and after administration of the combination therapy. Exemplary biological samples for use in the methods of the present disclosure include, without limitation, tumor samples, blood samples, serum samples, surgical samples, and biopsy samples. In one example, a bulk tissue sample can be subjected to whole exome and transcriptome analysis using any of known techniques in the art (e.g., the ImmunoID NEXT platform, Personalis, Inc.). The resulting data can be used for gene expression quantification. Whole transcriptome sequencing results can be aligned using e.g., STAR, and normalized expression value in transcripts per million (TPM) can be calculated using e.g., Personalis' ImmunoID NeXT tool, Expressionist. A hallmark gene signature score (e.g., a TNFα gene signature score, an E2F gene signature score, an IFN-γ gene signature score, or a MYC gene signature score) can be calculated for e.g., MYC, E2F, or IFN-γ by averaging the log normalized expression value for each gene in the MYC E2F, or IFN-γ hallmark gene set. For survival analysis, the subjects were stratified based on the value of this MYC E2F, or IFN-γ gene signature, where “high” vs “low” was defined by the median signature value across all subjects including the test subject and the one or more reference subjects. In some embodiments, lower normalized expression values of the set of genes in e.g., the MYC hallmark gene set can be significantly associated with longer overall survival in e.g., metastatic pancreatic cancer patients treated with e.g., sotigalimab in combination with gemcitabine+nab-Paclitaxel. In other embodiments, lower normalized expression values of the set of genes in e.g., an E2F gene set can be significantly associated with longer overall survival in e.g., metastatic pancreatic cancer patients treated with e.g., sotigalimab in combination with gemcitabine+nab-Paclitaxel. In some embodiments, increased normalized expression values of the set of genes in e.g., an IFNγ gene set can be significantly associated with longer overall survival in e.g., metastatic pancreatic cancer patients treated with e.g., sotigalimab in combination with gemcitabine+nab-Paclitaxel

A biological sample, e.g., a peripheral blood sample, can be obtained from the test subject and the one or more reference subjects both prior to and after administration of the combination therapy. Peripheral blood mononuclear cells (PBMCs) can be isolated from the peripheral blood sample. The isolated PBMCs can be subjected to immune profiling using e.g., the X50 Platform. For example, a multiplex flow panel designed to evaluate T cell phenotype and function can be utilized. In some embodiments, PBMCs can be identified as live CD45⁺ cells. The patient PBMCs can be classified into different immune cell populations based on the presence of surface markers. CD8⁺ T cells can be selected from CD45⁺ cells by the presence of CD3 and CD8 surface markers. CD4⁺ T cells can be selected from CD45⁺ cells by the presence of CD3 and CD8 surface markers. CD8+ and CD4⁺ T cells can be further subdivided into numerous T cell subsets, such as Effector Memory Type 1 (EM1) cells. EM1 T cells can be classically defined as CD45RA⁻CD27+CCR7−. This cell population can be further categorized by CXCR5 expression into a CXCR5⁺ population or CD244 expression into a CD244⁺ (also referred to as 2B4) population. The ratio of cell counts in this CXCR5⁺ population and/or CD244⁺ population to the total EM1 T cell population count can be shown to be associated with overall survival. In some embodiments, lower ratios of circulating CXCR5⁺ Effector Memory (CD45RA⁻CD27⁺) CD8⁺ T cells to total Effector Memory (CD45RA⁻CD27⁺) CD8⁺ T cells can be significantly associated with longer overall survival in e.g., metastatic pancreatic cancer patients treated with e.g., sotigalimab in combination with gemcitabine+nab-Paclitaxel. In some embodiments, lower ratios of circulating CD244⁺ Effector Memory (CD45RA⁻CD27⁺) CD4⁺ T cells to total Effector Memory (CD45RA⁻−D27+) CD4⁺ T cells can be significantly associated with longer overall survival in e.g., metastatic pancreatic cancer patients treated with e.g., sotigalimab in combination with gemcitabine+nab-Paclitaxel.

In other embodiments, CD4 T cells can be further characterized into Type I helper CD4 T cells and antigen-experienced CD4 T cells. Type I helper CD4 T cells can be identified by the expression of Tbet+, Eomes+, and PD-1+, whereas antigen-experienced CD4 T cells can be identified by the expression of PD-1+, Tbet+, and TCF-1+. Both of these populations can be significantly associated with longer overall survival in e.g., metastatic pancreatic cancer patients treated with e.g., sotigalimab in combination with gemcitabine+nab-Paclitaxel.

B cell phenotype and function can also be analyzed. B cells can be identified based on CD19 expression and further distinguished into memory vs naïve vs plasmablast based on expression of CD38 vs CD27. In some embodiments, cell counts of circulating HLA-DR+CCR7+B cells can be determined from a biological sample from a subject. The cell counts of this circulating B cells population can be compared to a control or reference sample, and, in some embodiments, increased cell counts of HLA-DR+CCR7+B cells can be significantly associated with longer overall survival in e.g., metastatic pancreatic cancer patients treated with e.g., sotigalimab in combination with gemcitabine+nab-Paclitaxel.

In some embodiments, cell counts of circulating cross-presenting dendritic cells (DCs) can be determined from a biological sample from a subject. As used herein, a cross-presenting dendritic cell can be any dendritic cell that acquires exogenous antigens for presentation on MHC class I molecules. Cross-presenting dendritic cells can be identified, for example, as described in the Examples infra. In some embodiments, cross-presenting dendritic cells can be identified by HLA-DR+CD14-CD16-CD11c+CD141+ markers. In some embodiments, the cross-presenting DCs are CD1C+CD141+. The cell counts of circulating cross-presenting DCs are compared to a control or reference sample, and, in some embodiments, increased cell counts of cross-presenting DCs or CD1C+CD141+ DCs can be significantly associated with longer overall survival in e.g., metastatic pancreatic cancer patients treated with e.g., sotigalimab in combination with gemcitabine+nab-Paclitaxel.

In the above method, the pre-treatment biological sample can be taken at any time point prior to treatment with the combination therapy of a CD40 agonist (e.g., sotigalimab)+chemotherapy (e.g., gemcitabine and nab-paclitaxel). For example, the pre-treatment biological sample can be taken minutes, hours, days, weeks, or months before initiation of the treatment, or substantially at the same time as the initiation of the treatment. The post-treatment biological sample can also be taken from the subject at any time point after initiation of treatment. For example, the post-treatment biological sample can be taken minutes, hours, days, weeks, or months after treatment with the combination therapy of a CD40 agonist (e.g., sotigalimab)+chemotherapy (e.g., gemcitabine and nab-paclitaxel). Non-limiting examples of the time points when the post-treatment biological sample is taken includes but is not limited to: 1 week to 24 months after, 1 week to 18 months after, 1 week to 12 months after, 1 week to 9 months after, 1 week to 6 months after, 1 week to 3 months after, 1 week to 9 weeks after, 1 week to 8 weeks after, 1 week to 6 weeks after, 1 week to 4 weeks after, or 1 week to 2 weeks after initiation of treatment with the combination therapy of a CD-40 agonist (e.g., sotigalimab)+chemotherapy (e.g., gemcitabine and nab-paclitaxel). The time points when the post-treatment biological sample can be taken is determined based on the cycle of the combination therapy. Non-limiting examples of such time points are: after 1st, 2nd, 3rd, 4th, 5th, 6th, 7th, 8th, 9th, 10th, 12th, 16th, 18th, 20th, 24th, 30th, or 32nd cycles.

A subject having been diagnosed with cancer can be determined to respond to a combination therapy including a CD40 agonist (e.g., anti-CD40 antibody such as sotigalimab) and chemotherapy, if the subject shows a partial response post treatment with the therapy. “Partial Response” means at least 30% decrease in the sum of the longest diameter (LD) of target lesions, taking as reference the baseline summed LD. A subject also can be determined to respond to the combination therapy, if the subject shows tumor shrinkage post-treatment with the therapy. A subject can be determined to respond to the combination therapy, if the subject shows progression free survival. “Progression Free Survival” (PFS) refers to the period from start date of treatment to the last date before entering Progressive Disease (PD) status. “PD” means at least 20% increase in the sum of the LD of target lesions, taking as reference the smallest summed LD recorded since the treatment started, or the appearance of one or more new lesions.

The biological samples can be obtained from a subject, e.g., a subject having, suspected of having, or at risk of developing cancer selected from, but not limited to, a pancreatic cancer, an endometrial cancer, a non-small cell lung cancer (NSCLC), a renal cell carcinoma ((RCC), e.g. clear cell RCC, non-clear cell RCC), a urothelial cancer, a head and neck cancer (e.g. head and neck squamous cell cancer), a melanoma (e.g., advanced melanoma such as Stage III-IV high-risk melanoma, unresectable or metastatic melanoma), a bladder cancer, a hepatocellular carcinoma, a breast cancer (e.g., triple negative breast cancer, ER⁺/HER2⁻ breast cancer), an ovarian cancer, a gastric cancer (e.g. metastatic gastric cancer or gastroesophageal junction adenocarcinoma), a colorectal cancer, a glioblastoma, a biliary tract cancer, a glioma (e.g., recurrent malignant glioma with a hypermutator phenotype), Merkel cell carcinoma (e.g., advanced or metastatic Merkel cell cancer), Hodgkin lymphoma, non-Hodgkin lymphoma (e.g. primary mediastinal B-cell lymphoma (PMBCL)), a cervical cancer, an advanced or refractory solid tumor, a small cell lung cancer (e.g., stage IV non-small cell lung cancer), a non-squamous non-small cell lung cancer, desmoplastic melanoma, a pediatric advanced solid tumor or lymphoma, a mesothelin-positive pleural mesothelioma, an esophageal cancer, an anal cancer, a salivary cancer, a prostate cancer, a carcinoid tumor, a primitive neuroectodermal tumor (pNET), and a thyroid cancer.

### III. Methods of the Treating Cancer

The methods provided herein can enable the assessment of a subject for responsiveness to a combination therapy including a CD40 agonist (e.g., anti-CD40 antibody such as sotigalimab) and chemotherapy. A subject who is likely to respond to the combination therapy can be administered e.g., sotigalimab and at least one chemotherapy drug (e.g., gemcitabine and nab-paclitaxel).

The methods of present disclosure can also enable the classification of subjects into groups of subjects that are more likely to benefit, and groups of subjects that are less likely to benefit, from treatment with the combination therapy with a CD40 agonist and chemotherapy. The ability to select such subjects from a pool of subjects who are being considered for the combination therapy including a CD40 agonist (e.g., anti-CD40 antibody such as sotigalimab) and chemotherapy is beneficial for effective treatment.

The methods provided herein can also be used to determine whether to continue the combination therapy including a CD40 agonist (e.g., anti-CD40 antibody such as sotigalimab) and chemotherapy after administering this therapy for a short period of time and determining based on the MYC gene signature score, E2F gene signature, IFN-γ gene signature, ratios of the circulating CXCR5⁺ effector memory CD8⁺ T cell to the total effector memory CD8⁺ T cell post-treatment versus pre-treatment, baseline levels of exhausted CD244⁺ effector memory CD4⁺ T cells, baseline levels of CXCR5⁺ effector memory CD8⁺ T cells, baseline levels of circulating cross-presenting dendritic cells, baseline levels of CD1C+CD141+ dendritic cells, h baseline circulating HLA-DR+CCR7+B cells, baseline circulating PD-1+ T cells, circulating TCF-1+ T cells, and/or circulating Tbet+ T cells, levels of circulating T helper cells, or any combination thereof whether this therapy is more likely or less likely to benefit the patient.

If the subject is more likely to respond to the combination therapy including a CD40 agonist (e.g., anti-CD40 antibody such as sotigalimab) and chemotherapy, the subject can then be administered an effective amount of one or more chemotherapy drugs (e.g., gemcitabine, nab-paclitaxel) and a CD40 agonist (e.g., a CD40 antibody such as sotigalimab). An effective amount of each chemotherapy drug and the CD40 agonist can suitably be determined by a health care practitioner taking into account, for example, the characteristics of the patient (e.g., age, sex, weight, race, etc.), the progression of the disease, and prior exposure to the drug.

In some embodiments, a CD40 agonist is an anti-CD40 antibody. In some embodiments, the anti-CD40 antibody is selected from the group consisting of sotigalimab, selicrelumab, ChiLob7/4. ADC-1013, SEA-CD40, CP-870,893, dacetuzumab, and CDX-1140. In some embodiments, the anti-CD40 antibody is sotigalimab.

In some embodiments, the method can include administering 240 mg of sotigalimab to the patient about every two weeks.

In some embodiment, one or more chemotherapy drugs can be selected from the group consisting of gemcitabine, nab-paclitaxel, folfirionx, nitrogen mustard/oxazaphosphorine, nitrosourea, triazene, and alkyl sulfonates, anthracycline antibiotics such as doxorubicin and daunorubicin, taxanes such as Taxol brand and docetaxel, vinca alkaloids such as vincristine and vinblastine, 5-fluorouracil (5-FU), leucovorin, Irinotecan, idarubicin, mitomycin C, oxaliplatin, raltitrexed, pemetrexed, tamoxifen, cisplatin, carboplatin, methotrexate, a Tinomycin D, mitoxantrone, brenoxane, mitramycin, methotrexate, paclitaxel, 2-methoxyestradiol, purinomastert, batimastat, BAY 12-9656, carboxamidotriazole, CC-1088, dextromethorphan acetic acid, dimethylxanthenone acetic acid, Endostatin, IM-862, marimastat, penicillamine, PTK787/ZK 222584, RPI. 4610, squalamine lactate, SU5416, thalidomide, combretastatin, tamoxifen, COL-3, neobasstat, BMS-275291, SU6668, anti-VEGF antibody, Med-522 (Vitaxin II), CAI, interleukin 12, IM862, amiloride, Angiostatin, angiostatin Kl-3, angiostatin K1-5, captopril, DL-α-difluoromethylornithine, DL-α-difluoromethylornithine HCl, endostatin, fumagillin, herbimycin A, 4-hydroxyphenylretinamide, Juglone, laminin, laminin hexapeptide, laminin pentapeptide, labendustin A, medroxyprogesterone, minocycline, placental ribonuclease Inhibitors, suramin, thrombospondin, antibodies targeting pro-angiogenic factors, topoisomerase inhibitors, microtubule inhibitors, low-molecular-weight tyrosine kinase inhibitors of pro-angiogenic growth factors Agents, GTPase inhibitors, histone deacetylase inhibitors, AKT kinase or ATPase inhibitors, Win (Wnt) signal inhibitors, E2F transcription factor inhibitors, mTOR inhibitors Agents, α, β and γ interferons, IL-12, matrix metalloproteinase inhibitors, ZD6474, SU1248, vitaxin, PDGFR inhibitors, NM3 and 2-ME2, and sirengitide. In some embodiments, the one or more chemotherapy drugs can be a combination of gemcitabine and nab-paclitaxel.

After classifying or selecting a subject based on whether the subject will be more likely or less likely to respond to the combination therapy including a CD40 agonist (e.g., anti-CD40 antibody such as sotigalimab) and chemotherapy, a medical practitioner (e.g., a doctor) can administer the appropriate therapeutic modality to the subject. Methods of administering an anti-CD40 antibody (e.g., sotigalimab, selicrelumab, ChiLob7/4. ADC-1013, SEA-CD40, CP-870,893, dacetuzumab, and CDX-1140) are well known in the art, e.g. described in their product labels

It is understood that any therapy described herein (e.g., a combination therapy including a CD40 agonist (e.g., anti-CD40 antibody such as sotigalimab) and chemotherapy or a therapy other than the combination therapy) can include one or more additional therapeutic agents. That is, any therapy described herein can be co-administered (administered in combination) with one or more additional anti-tumor agents. Furthermore, any therapy described herein can include one or more agents for treating, for example, pain, nausea, and/or one or more side-effects of the combination therapy including a CD40 agonist (e.g., anti-CD40 antibody such as sotigalimab) and chemotherapy.

The combination therapy including a CD40 agonist (e.g., anti-CD40 antibody such as sotigalimab) and chemotherapy can be, e.g., simultaneous or successive. For example, one or more chemotherapy drugs and an anti-CD40 antibody can be administered at the same time or one or more chemotherapy drugs can be administered first in time and an anti-CD40 antibody administered second in time, or vice versa. The dosing frequency of the one or more chemotherapy drugs and the anti-CD40 antibody can be different or same. In one embodiment, the dosing frequency is different. An exemplary dosing frequency of the combination therapy including a CD40 agonist (e.g., anti-CD40 antibody such as sotigalimab) and chemotherapy can be once in a few weeks, for example, 1 week, 2 weeks, 3 weeks, 4 weeks or 1 month, or 6 weeks.

### IV. Therapeutic Applications

### A. Administration of Antibodies

The antibodies described herein are administered in an effective regime meaning a dosage, route of administration and frequency of administration that delays the onset, reduces the severity, inhibits further deterioration, and/or ameliorates at least one sign or symptom of a disorder. If a subject is already suffering from a disorder, the regime can be referred to as a therapeutically effective regime. If the subject is at elevated risk of the disorder relative to the general population but is not yet experiencing symptoms, the regime can be referred to as a prophylactically effective regime. In some instances, therapeutic or prophylactic efficacy can be observed in an individual subject relative to historical controls or past experience in the same subject. In other instances, therapeutic or prophylactic efficacy can be demonstrated in a preclinical or clinical trial in a population of treated subjects relative to a control population of untreated subjects.

In some instances, the subject is identified as PD-L1 positive, CD40 positive, having lower baseline levels exhausted CD244+ effector memory CD4+ T cells, lower baseline levels of CXCR5+ effector memory CD8+ T cells, higher baseline levels of circulating cross-presenting dendritic cells, higher baseline levels of CD1C+CD141+ dendritic cells, higher levels of baseline circulating HLA-DR+CCR7+B cells, higher levels of baseline circulating PD-1+ T cells, circulating TCF-1+ T cells, and/or circulating Thet+ T cells, higher levels of circulating T helper cells, or any combination thereof. In some embodiments, a patient is selected for treatment with the antibodies described herein based on low baseline expression of one or more genes from a MYC gene set, or an E2F gene set, relative to a reference population. In some instances, one or more genes is selected from the group consisting of ABCE1, ACP1, AIMP2, AP3S1, APEX1, BUB3, C1QBP, CAD, CANX, CBX3, CCNA2, CCT2, CCT3, CCT4, CCT5, CCT7, CDC20, CDC45, CDK2, CDK4, CLNS1A, CNBP, COPS5, COX5A, CSTF2, CTPS1, CUL1, CYC1, DDX18, DDX21, DEK, DHX15, DUT, EEF1B2, EIF1AX, EIF2S1, EIF2S2, EIF3B, EIF3D, EIF3J, EIF4A1, EIF4E, EIF4G2, EIF4H, EPRS1, ERH, ETF1, EXOSC7, FAM120A, FBL, G3BP1, GLO1, GNL3, GOT2, GSPT1, H2AZ1, HDAC2, HDDC2, HDGF, HNRNPA1, HNRNPA2B1, HNRNPA3, HNRNPC, HNRNPD, HNRNPR, HNRNPU, HPRT1, HSP90AB1, HSPD1, HSPE1, IARS1, IFRD1, ILF2, IMPDH2, KARS1, KPNA2, KPNB1, LDHA, LSM2, LSM7, MAD2L1, MCM2, MCM4, MCM5, MCM6, MCM7, MRPL23, MRPL9, MRPS18B, MYC, NAP1L1, NCBP1, NCBP2, NDUFAB1, NHP2, NME1, NOLC1, NOP16, NOP56, NPM1, ODC1, ORC2, PA2G4, PABPC1, PABPC4, PCBP1, PCNA, PGK1, PHB, PHB2, POLD2, POLE3, PPIA, PPM1G, PRDX3, PRDX4, PRPF31, PRPS2, PSMA1, PSMA2, PSMA4, PSMA6, PSMA7, PSMB2, PSMB3, PSMC4, PSMC6, PSMD1, PSMD14, PSMD3, PSMD7, PSMD8, PTGES3, PWP1, RACK1, RAD23B, RAN, RANBP1, RFC4, RNPS1, RPL14, RPL18, RPL22, RPL34, RPL6, RPLP0, RPS10, RPS2, RPS3, RPS5, RPS6, RRM1, RRP9, RSL1D1, RUVBL2, SERBP1, SET, SF3A1, SF3B3, SLC25A3, SMARCC1, SNRPA, SNRPA1, SNRPB2, SNRPD1, SNRPD2, SNRPD3, SNRPG, SRM, SRPK1, SRSF1, SRSF2, SRSF3, SRSF7, SSB, SSBP1, STARD7, SYNCRIP, TARDBP, TCP1, TFDP1, TOMM70, TRA2B, TRIM28, TUFM, TXNL4A, TYMS, U2AF1, UBA2, UBE2E1, UBE2L3, USP1, VBP1, VDAC1, VDAC3, XPO1, XPOT, XRCC6, YWHAE, and YWHAQ. In some embodiments, a patient is selected for treatment with the antibodies described herein based on high baseline expression of one or more genes from an IFNγ gene set, relative to a reference population. In some instances, one or more genes is selected from the group consisting of CD8A, CD274, LAG3, and STAT1.

In some instances, the gene set further comprises E2F1-3.

In some aspects, any of the methods described herein include the administration of a therapeutically effective amount of one or more of the anti-CD40 antibodies described herein to subjects in need thereof. As used herein, a “therapeutically effective amount” or “therapeutically effective dosage” of an anticancer therapy (such as any of the anti-CD40 antibodies described herein) is an amount sufficient to effect beneficial or desired results. For therapeutic use, beneficial or desired results include but are not limited to clinical results such as decreasing one or more symptoms resulting from cancer, increasing the quality of life of subjects suffering from cancer, decreasing the dose of other medications required to treat the cancer, enhancing the effect of another medication such as via targeting, delaying the progression of the disease, and/or prolonging survival. An effective dosage can be administered in one or more administrations. For purposes of this disclosure, an effective dosage of an anti-cancer therapy is an amount sufficient to accomplish therapeutic or prophylactic treatment either directly or indirectly. As is understood in the clinical context, a therapeutically effective dosage of an anti-cancer therapy may or may not be achieved in conjunction with another anti-cancer therapy.

Exemplary dosages for any of the antibodies described herein are about 0.1-20 mg/kg or 0.5-5 mg/kg body weight (e.g., about 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg) or 10-1600 mg (such as any of less than 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, or 1600 mg or greater, inclusive of values in between these numbers), as a fixed dosage. In one embodiment, the antibody described herein in given in an amount of about 300 to 1500 mg every three weeks. In another embodiment, the antibody described herein is given in an amount of about 300 to 1800 mg every four weeks. The dosage depends on the condition of the subject and response to prior treatment, if any, whether the treatment is prophylactic or therapeutic and whether the disorder is acute or chronic, among other factors.

Administration can be parenteral, intravenous, oral, subcutaneous, intra-arterial, intracranial, intrathecal, intraperitoneal, intratumoral, topical, intranasal or intramuscular. In some embodiments, administration into the systemic circulation is by intravenous or subcutaneous administration. Intravenous administration can be, for example, by infusion over a period such as 30-90 min.

The frequency of administration depends on the half-life of the antibody in the circulation, the condition of the subject and the route of administration among other factors. The frequency can be daily, weekly, monthly, quarterly, or at irregular intervals in response to changes in the subject's condition or progression of the disorder being treated. In an embodiment, the frequency can be in two-week cycles. In another embodiment, the frequency can be in three-week cycles. In another embodiment, the frequency is four-week cycles. In another embodiment, the frequency is six-week cycles. An exemplary frequency for intravenous administration is between weekly and quarterly over a continuous cause of treatment, although more or less frequent dosing is also possible. For subcutaneous administration, an exemplary dosing frequency is daily to monthly, although more or less frequent dosing is also possible.

The number of dosages administered depends on whether the disorder is acute or chronic and the response of the disorder to the treatment. For acute disorders or acute exacerbations of chronic disorders between 1 and 10 doses are often sufficient. Sometimes a single bolus dose, optionally in divided form, is sufficient for an acute disorder or acute exacerbation of a chronic disorder. Treatment can be repeated for recurrence of an acute disorder or acute exacerbation. For chronic disorders, an antibody can be administered at regular intervals, e.g., weekly, fortnightly, monthly, quarterly, every six months for at least 1, 5 or 10 years, or the life of the subject.

Treatment including an anti-CD40 antibody can alleviate a disease by increasing the median progression-free survival or overall survival time of subjects with cancer by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100%, compared to control subjects, or increase either of these times by 2 weeks, 1, 2 or 3 months, or by 4 or 6 months or even 9 months or a year. In addition or alternatively, treatment including the anti-CD40 antibody can increase the complete response rate, partial response rate, or objective response rate (complete+partial) of subjects by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% compared to the control subjects. Control subjects receive the same treatment as subjects receiving the anti-CD40 antibody except for the anti-CD40 antibody. Thus, control subjects can receive placebo alone or a combination of placebo and some chemotherapeutic agent other than the anti-CD40 antibody if such is also received by the subjects receiving the anti-CD40 antibody.

The anti-CD40 antibodies disclosed herein can enhance the number of activated effector memory T cells (Ki67+CD8+) relative to the amount of effector memory T cells (Ki67+CD8+) in the absence of one of the anti-CD40 antibodies disclosed herein. The anti-CD40 antibodies disclosed herein can also enhance the number of activated myeloid dendritic cells (CD86+) relative to the amount of activated myeloid dendritic cells (CD86+) in the absence of one of the anti-CD40 antibodies disclosed herein. The anti-CD40 antibodies disclosed herein can further increase the amount of tumoral CD80+M1 macrophages.

The anti-CD40 antibodies can also decrease bacteroidia and increase clostridia as well as gammaproteobacteria in stool samples of subjects as compared to control subjects.

Typically, in a clinical trial (e.g., a phase II, phase II/III or phase III trial), increases in median progression-free survival and/or response rate of the subjects treated with the anti-CD40 antibody, relative to the control group of subjects are statistically significant, for example, at the p=0.05 or 0.01 or even 0.001 level. The complete and partial response rates are determined by objective criteria commonly used in clinical trials for cancer, e.g., as listed or accepted by the National Cancer Institute and/or Food and Drug Administration and can include for example, tumor volume, number of tumors, metastasis, survival time, and quality of life measures, among others.

Pharmaceutical compositions for parenteral administration can be sterile and substantially isotonic and manufactured under GMP conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, such as in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively, antibodies can be in lyophilized form for constitution with a suitable vehicle, e.g., sterile pyrogen-free water, before use. The concentration of antibody in liquid formulations can vary from e.g., about 10-150 mg/ml. In some formulations the concentration is about 20-80 mg/ml.

### B. Combination Therapies

The present disclosure contemplates the use of anti-CD40 antibody alone or in combination with one or more active therapeutic agents. The additional active therapeutic agents can be small chemical molecules; macromolecules such as proteins, antibodies, peptibodies, peptides, DNA, RNA or fragments of such macromolecules; or cellular or gene therapies. The combination therapy can target different, but complementary, mechanisms of action and thereby have a synergistic therapeutic or prophylactic effect on the underlying disease, disorder, or condition. In addition, or alternatively, the combination therapy can allow for a dose reduction of one or more of the agents, thereby ameliorating, reducing or eliminating adverse effects associated with one or more of the agents.

The active therapeutic agents in such combination therapy can be formulated as a single composition or as separate compositions. If administered separately, each therapeutic agent in the combination can be given at or around the same time, or at different times. Furthermore, the therapeutic agents are administered “in combination” even if they have different forms of administration (e.g., oral capsule and intravenous), they are given at different dosing intervals, one therapeutic agent is given at a constant dosing regimen while another is titrated up, titrated down or discontinued, or each therapeutic agent in the combination is independently titrated up, titrated down, increased or decreased in dosage, or discontinued and/or resumed during a patient's course of therapy. If the combination is formulated as separate compositions, in some embodiments, the separate compositions are provided together in a kit.

In certain embodiments, any of the anti-CD40 antibodies disclosed herein are administered or applied sequentially to one or more of the additional active therapeutic agents, e.g., where one or more of the additional active therapeutic agents is administered prior to or after the administration of the anti-CD40 antibody according to this disclosure. In other embodiments, the antibodies are administered simultaneously with one or more of the additional active therapeutic agents, e.g., where the anti-CD40 antibody is administered at or about the same time as one or more of the additional therapeutic agents; the anti-CD40 antibody and one or more of the additional therapeutic agents can be present in two or more separate formulations or combined into a single formulation (i.e., a co-formulation). Regardless of whether the additional agent(s) are administered sequentially or simultaneously with the anti-CD40 antibody, they are considered to be administered in combination for purposes of the present disclosure.

The antibodies of the present disclosure can be used in combination with at least one other (active) agent in any manner appropriate under the circumstances. In one embodiment, treatment with the at least one active agent and at least one anti-CD40 antibody of the present disclosure is maintained over a period of time. In another embodiment, treatment with the at least one active agent is reduced or discontinued (e.g., when the subject is stable), while treatment with an anti-CD40 antibody of the present disclosure is maintained at a constant dosing regimen. In a further embodiment, treatment with the at least one active agent is reduced or discontinued (e.g., when the subject is stable), while treatment with an anti-CD40 antibody of the present disclosure is reduced (e.g., lower dose, less frequent dosing or shorter treatment regimen). In yet another embodiment, treatment with the at least one active agent is reduced or discontinued (e.g., when the subject is stable), and treatment with the anti-CD40 antibody of the present disclosure is increased (e.g., higher dose, more frequent dosing or longer treatment regimen). In yet another embodiment, treatment with the at least one active agent is maintained and treatment with the anti-CD40 antibody of the present disclosure is reduced or discontinued (e.g., lower dose, less frequent dosing or shorter treatment regimen). In yet another embodiment, treatment with the at least one active agent and treatment with the anti-CD40 antibodies of the present disclosure are reduced or discontinued (e.g., lower dose, less frequent dosing or shorter treatment regimen).

Treatment with antibodies of the present disclosure can be combined with other treatments effective against the disorder being treated. When used in treating a proliferative condition, cancer, tumor, or precancerous disease, disorder or condition, the antibodies of the present disclosure can be combined with chemotherapy, radiation (e.g., localized radiation therapy or total body radiation therapy), stem cell treatment, surgery or treatment with other biologics.

Antibodies of the present disclosure can be administered with vaccines eliciting an immune response against a cancer. Such immune response is enhanced by the antibody of the present disclosure. The vaccine can include an antigen expressed on the surface of the cancerous cell and/or tumor of a fragment thereof effective to induce an immune response, optionally linked to a carrier molecule.

In some embodiments, one or more of the additional therapeutic agents is an immunomodulatory agent. Suitable immunomodulatory agents that can be used in the present disclosure include CD40L, B7, and B7RP1; activating monoclonal antibodies (mAbs) to stimulatory receptors, such as, anti-CD38, anti-ICOS, and 4-IBB ligand; dendritic cell antigen loading (in vitro or in vivo); anti-cancer vaccines such as dendritic cell cancer vaccines; cytokines/chemokines, such as, IL1, IL2, IL12, IL18, ELC/CCL19, SLC/CCL21, MCP-1, IL-4, IL-18, TNF, IL-15, MDC, IFNα/β, M-CSF, IL-3, GM-CSF, IL-13, and anti-IL-10; bacterial lipopolysaccharides (LPS); indoleamine 2,3-dioxygenase 1 (IDO1) inhibitors and immune-stimulatory oligonucleotides.

In certain embodiments, the present disclosure provides methods for suppression of tumor growth including administration of an anti-CD40 antibody described herein in combination with a signal transduction inhibitor (STI) to achieve additive or synergistic suppression of tumor growth. As used herein, the term “signal transduction inhibitor” refers to an agent that selectively inhibits one or more steps in a signaling pathway. Signal transduction inhibitors (STIs) contemplated by the present disclosure include: (i) bcr/abl kinase inhibitors (e.g., imatinib mesylate, GLEEVEC®); (ii) epidermal growth factor (EGF) receptor inhibitors, including kinase inhibitors (e.g., gefitinib, erlotinib, afatinib and osimertinib) and antibodies; (iii) her-2/neu receptor inhibitors (e.g., HERCEPTIN®); (iv) inhibitors of Akt family kinases or the Akt pathway (e.g., rapamycin); (v) cell cycle kinase inhibitors (e.g., flavopiridol); and (vi) phosphatidyl inositol kinase inhibitors. Agents involved in immunomodulation can also be used in combination with the anti-TIGIT antibody described herein for the suppression of tumor growth in cancer patients.

In some embodiments, one or more of the additional therapeutic agents is a chemotherapeutic agent. Examples of chemotherapeutic agents include, but are not limited to, gemcitabine, nab-paclitaxel, folfirionx, nitrogen mustard/oxazaphosphorine, nitrosourea, triazene, and alkyl sulfonates, anthracycline antibiotics such as doxorubicin and daunorubicin, taxanes such as Taxol brand and docetaxel, vinca alkaloids such as vincristine and vinblastine, 5-fluorouracil (5-FU), leucovorin, Irinotecan, idarubicin, mitomycin C, oxaliplatin, raltitrexed, pemetrexed, tamoxifen, cisplatin, carboplatin, methotrexate, a Tinomycin D, mitoxantrone, brenoxane, mitramycin, methotrexate, paclitaxel, 2-methoxyestradiol, purinomastert, batimastat, BAY 12-9656, carboxamidotriazole, CC-1088, dextromethorphan acetic acid, dimethylxanthenone acetic acid, Endostatin, IM-862, marimastat, penicillamine, PTK787/ZK 222584, RPI. 4610, squalamine lactate, SU5416, thalidomide, combretastatin, tamoxifen, COL-3, ncobasstat, BMS-275291, SU6668, anti-VEGF antibody, Med-522 (Vitaxin II), CAI, interleukin 12, IM862, amiloride, Angiostatin, angiostatin Kl-3, angiostatin K1-5, captopril, DL-α-difluoromethylornithine, DL-α-difluoromethylornithine HCl, endostatin, fumagillin, herbimycin A, 4-hydroxyphenylretinamide, Juglone, laminin, laminin hexapeptide, laminin pentapeptide, labendustin A, medroxyprogesterone, minocycline, placental ribonuclease Inhibitors, suramin, thrombospondin, antibodies targeting pro-angiogenic factors, topoisomerase inhibitors, microtubule inhibitors, low-molecular-weight tyrosine kinase inhibitors of pro-angiogenic growth factors Agents, GTPase inhibitors, histone deacetylase inhibitors, AKT kinase or ATPase inhibitors, Win (Wnt) signal inhibitors, E2F transcription factor inhibitors, mTOR inhibitors Agents, α, β and γ interferons, IL-12, matrix metalloproteinase inhibitors, ZD6474, SU1248, vitaxin, PDGFR inhibitors, NM3 and 2-ME2, and sirengitide; and pharmaceutically acceptable salts, acids or derivatives of any of the above.

Chemotherapeutic agents also include anti-hormonal agents that act to regulate or inhibit hormonal action on tumors such as anti-estrogens, including, for example, tamoxifen, raloxifene, aromatase inhibiting 4(5)-imidazoles, 4-hydroxytamoxifen, trioxifene, keoxifene, onapristone, and toremifene; and antiandrogens such as abiraterone, enzalutamide, apalutamide, darolutamide, flutamide, nilutamide, bicalutamide, leuprolide, and goserelin; and pharmaceutically acceptable salts, acids or derivatives of any of the above. In certain embodiments, combination therapy includes a chemotherapy regimen that includes one or more chemotherapeutic agents. In certain embodiments, combination therapy includes administration of a hormone or related hormonal agent.

Additional treatment modalities that can be used in combination with an anti-CD40 antibody include radiotherapy, an antibody against a tumor antigen, a complex of an antibody and toxin, a T cell adjuvant, bone marrow transplant, or antigen presenting cells (e.g., dendritic cell therapy), including TLR agonists which are used to stimulate such antigen presenting cells.

In certain embodiments, the present disclosure contemplates the use of the anti-CD40 antibody described herein in combination with RNA interference-based therapies to silence gene expression. RNAi begins with the cleavage of longer double-stranded RNAs into small interfering RNAs (siRNAs). One strand of the siRNA is incorporated into a ribonucleoprotein complex known as the RNA-induced silencing complex (RISC), which is then used to identify mRNA molecules that are at least partially complementary to the incorporated siRNA strand. RISC can bind to or cleave the mRNA, both of which inhibits translation.

In certain embodiments, the present disclosure contemplates the use of the anti-CD40 antibody described herein in combination with agents that modulate the level of adenosine. Such therapeutic agents can act on the ectonucleotides that catalyze the conversion of ATP to adenosine, including ectonucleoside triphosphate diphosphohydrolase 1 (ENTPD1, also known as CD39 or Cluster of Differentiation 39), which hydrolyzes ATP to ADP and ADP to AMP, and 5′-nucleotidase, ecto (NT5E or 5NT, also known as CD73 or Cluster of Differentiation 73), which converts AMP to adenosine. In one embodiment, the present disclosure contemplates combination with CD73 inhibitors such as those described in WO 2017/120508, WO 2018/094148 and WO 2018/067424. In one embodiment, the CD73 inhibitor is AB680. In another approach, adenosine A2a and A2b receptors are targeted. Combination with antagonists of the A2a and/or A2b receptors is also contemplated. In one embodiment, the present disclosure contemplates combination with the adenosine receptor antagonists described in WO/2018/136700 or WO 2018/204661. In one embodiment, the adenosine receptor antagonist is AB928 (etrumadenant).

In certain embodiments, the present disclosure contemplates the use of the anti-CD40 antibody described herein in combination with inhibitors of phosphatidylinositol 3-kinases (PI3Ks), particularly the PI3Kγ isoform. PI3Kγ inhibitors can stimulate an anti-cancer immune response through the modulation of myeloid cells, such as by inhibiting suppressive myeloid cells, dampening immune-suppressive tumor-infiltrating macrophages or by stimulating macrophages and dendritic cells to make cytokines that contribute to effective T cell responses leading to decreased cancer development and spread. Exemplary PI3Kγ inhibitors that can be combined with the anti-CD40 antibody described herein include those described in WO 2020/0247496A1. In one embodiment, the PI3Kγ inhibitor is IPI-549.

In certain embodiments, the present disclosure contemplates the use of the anti-CD40 antibody described herein in combination with inhibitors of arginase, which has been shown to be either responsible for or to participate in inflammation-triggered immune dysfunction, tumor immune escape, immunosuppression and immunopathology of infectious disease. Exemplary arginase compounds can be found, for example, in PCT/US2019/020507 and WO/2020/102646.

In certain embodiments, the present disclosure contemplates the use of the anti-CD40 antibody according to this disclosure with inhibitors of HIF-2α, which plays an integral role in cellular response to low oxygen availability. Under hypoxic conditions, the hypoxia-inducible factor (HIF) transcription factors can activate the expression of genes that regulate metabolism, angiogenesis, cell proliferation and survival, immune evasion, and inflammatory response. HIF-2α overexpression has been associated with poor clinical outcomes in patients with various cancers; hypoxia is also prevalent in many acute and chronic inflammatory disorders, such as inflammatory bowel disease and rheumatoid arthritis.

The present disclosure also contemplates the combination of the anti-CD40 antibody described herein with one or more RAS signaling inhibitors. Oncogenic mutations in the RAS family of genes, e.g., HRAS, KRAS, and NRAS, are associated with a variety of cancers. For example, mutations of G12C, G12D, G12V, G12A, G13D, Q61H, G13C and G12S, among others, in the KRAS family of genes have been observed in multiple tumor types. Direct and indirect inhibition strategies have been investigated for the inhibition of mutant RAS signaling. Indirect inhibitors target effectors other than RAS in the RAS signaling pathway, and include, but are not limited to, inhibitors of RAF, MEK, ERK, PI3K, PTEN, SOS (e.g., SOS1), mTORC1, SHP2 (PTPN11), and AKT. Non-limiting examples of indirect inhibitors under development include RMC-4630, RMC-5845, RMC-6291, RMC-6236, JAB-3068, JAB-3312, TNO155, RLY-1971, BI1701963. Direct inhibitors of RAS mutants have also been explored, and generally target the KRAS-GTP complex or the KRAS-GDP complex. Exemplary direct RAS inhibitors under development include, but are not limited to, sotorasib (AMG510), MRTX849, mRNA-5671 and ARS1620. In some embodiments, the one or more RAS signaling inhibitors are selected from the group consisting of RAF inhibitors, MEK inhibitors, ERK inhibitors, PI3K inhibitors, PTEN inhibitors, SOS1 inhibitors, mTORC1 inhibitors, SHP2 inhibitors, and AKT inhibitors. In other embodiments the one or more RAS signaling inhibitors directly inhibit RAS mutants.

In some embodiments, this disclosure is directed to the combination of the anti-CD40 antibody according to this disclosure with one or more inhibitors of anexelekto (i.e., AXL). The AXL signaling pathway is associated with tumor growth and metastasis, and is believed to mediate resistance to a variety of cancer therapies. There are a variety of AXL inhibitors under development that also inhibit other kinases in the TAM family (i.e., TYRO3, MERTK), as well as other receptor tyrosine kinases including MET, FLT3, RON and AURORA, among others. Exemplary multikinase inhibitors include gilteritinib, merestinib, cabozantinib, BMS777607, and forctinib. AXL specific inhibitors have also been developed, e.g., SGI-7079, TP-0903 (i.e., dubermatinib), BGB324 (i.e., bemcentinib) and DP3975.

In certain embodiments, the present disclosure contemplates the use of the anti-TIGIT antibody described herein in combination with adoptive cell therapy, a new and promising form of personalized immunotherapy in which immune cells with anti-tumor activity are administered to cancer patients. Adoptive cell therapy is being explored using tumor-infiltrating lymphocytes (TIL) and T cells engineered to express, for example, chimeric antigen receptors (CAR) or T cell receptors (TCR). Adoptive cell therapy generally involves collecting T cells from an individual, genetically modifying them to target a specific antigen or to enhance their anti-tumor effects, amplifying them to a sufficient number, and infusion of the genetically modified T cells into a cancer patient. T cells can be collected from the patient to whom the expanded cells are later reinfused (e.g., autologous) or can be collected from donor patients (e.g., allogeneic).

T cell-mediated immunity includes multiple sequential steps, each of which is regulated by counterbalancing stimulatory and inhibitory signals in order to optimize the response. While nearly all inhibitory signals in the immune response ultimately modulate intracellular signaling pathways, many are initiated through membrane receptors, the ligands of which are either membrane-bound or soluble (cytokines). While co-stimulatory and inhibitory receptors and ligands that regulate T cell activation are frequently not overexpressed in cancers relative to normal tissues, inhibitory ligands and receptors that regulate T cell effector functions in tissues are commonly overexpressed on tumor cells or on non-transformed cells associated with the tumor microenvironment. The functions of the soluble and membrane-bound receptor (ligand immune checkpoints) can be modulated using agonist antibodies (for co-stimulatory pathways) or antagonist antibodies (for inhibitory pathways). Thus, in contrast to most antibodies currently approved for cancer therapy, antibodies that block or agonize immune checkpoints do not target tumor cells directly, but rather target lymphocyte receptors or their ligands in order to enhance endogenous antitumor activity. [See Pardoll, (April 2012) Nature Rev. Cancer 12:252-64].

Examples of immune checkpoints (ligands and receptors), some of which are selectively upregulated in various types of tumor cells, that are candidates for blockade include PD-1 (programmed cell death protein 1); PD-L1 (programmed cell death 1 ligand 1); BTLA (B and T lymphocyte attenuator); CTLA4 (cytotoxic T-lymphocyte associated antigen 4); TIM-3 (T cell immunoglobulin mucin protein 3); LAG-3 (lymphocyte activation gene 3); TIGIT (T cell immunoreceptor with Ig and ITIM domains); and Killer Inhibitory Receptors, which can be divided into two classes based on their structural features: i) killer cell immunoglobulin-like receptors (KIRs), and ii) C-type lectin receptors (members of the type II transmembrane receptor family). Other less well-defined immune checkpoints have been described in the literature, including both receptors (e.g., the 2B4 (also known as CD244) receptor) and ligands (e.g., certain B7 family inhibitory ligands such B7-H3 (also known as CD276) and B7-H4 (also known as B7-S1, B7× and VCTN1)). [See Pardoll, (April 2012) Nature Rev. Cancer 12:252-64].

The present disclosure contemplates the use of the anti-CD40 antibody described herein in combination with inhibitors of the aforementioned immune-checkpoint receptors and ligands, as well as yet-to-be-described immune-checkpoint receptors and ligands. Certain modulators of immune checkpoints are currently approved, and many others are in development. When it was approved for the treatment of melanoma in 2011, the fully humanized CTLA4 monoclonal antibody ipilimumab (e.g., YERVOY®; Bristol Myers Squibb) became the first immune checkpoint inhibitor to receive regulatory approval in the US. Fusion proteins including CTLA4 and an antibody (CTLA4-Ig; abatcept (e.g., ORENCIA®; Bristol Myers Squibb)) have been used for the treatment of rheumatoid arthritis, and other fusion proteins have been shown to be effective in renal transplantation patients that are sensitized to Epstein Barr Virus. The next class of immune checkpoint inhibitors to receive regulatory approval were against PD-1 and its ligands PD-L1 and PD-L2. Approved anti-PD-1 antibodies include nivolumab (e.g., OPDIVO®; Bristol Myers Squibb) and pembrolizumab (e.g., KEYTRUDA®; Merck) for various cancers, including squamous cell carcinoma, classical Hodgkin lymphoma and urothelial carcinoma. Approved anti-PD-L1 antibodies include avelumab (e.g., BAVENCIO®; EMD Serono & Pfizer), atezolizumab (e.g., TECENTRIQ®; Roche/Genentech), and durvalumab (e.g., IMFINZI®; AstraZeneca) for certain cancers, including urothelial carcinoma. In some combinations provided herein, the immune checkpoint inhibitor is selected from MEDI-0680 nivolumab, pembrolizumab, avelumab, atezolizumab, budigalimab, BI-754091, camrelizumab, cosibelimab, durvalumab, dostarlimab, cemiplimab, sintilimab, tislelizumab, toripalimab, retifanlimab, sasanlimab, and zimberelimab (AB122). In some embodiments, the immune checkpoint inhibitor is MEDI-0680 (AMP-514; WO2012/145493) or pidilizumab (CT-011). Another approach to target the PD-1 receptor is the recombinant protein composed of the extracellular domain of PD-L2 (B7-DC) fused to the Fc portion of IgG1, called AMP-224. In one embodiment, the present disclosure contemplates the use of an anti-CD40 antibody according to this disclosure with a PD-1 antibody. In one particular embodiment, the PD-1 antibody is nivolumab.

In another aspect, the present disclosure contemplates combination with a cytokine that inhibits T cell activation (e.g., IL-6, IL-10, TGF-B, VEGF, and other immunosuppressive cytokines) or a cytokine that stimulates T cell activation, for stimulating an immune response.

In yet another aspect, T cell responses can be stimulated by a combination of the disclosed anti-CD40 antibody and one or more of (i) an antagonist of a protein that inhibits T cell activation (e.g., immune checkpoint inhibitors) such as CTLA-4, PD-1, PD-L1, PD-L2, LAG-3, TIM-3, PVRIG, Galectin 9, CEACAM-1, BTLA, CD69, Galectin-1, CD113, GPR56, VISTA, 2B4, CD48, GARP, PD1H, LAIR1, TIM-1, and TIM-4, and/or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD2. Other agents that can be combined with the anti-CD40 antibody of the present disclosure for the treatment of cancer include antagonists of inhibitory receptors on NK cells or agonists of activating receptors on NK cells. For example, the anti-CD40 antibody described herein can be combined with antagonists of KIR, such as lirilumab.

Yet other agents for combination therapies include agents that inhibit or deplete macrophages or monocytes, including but not limited to CSF-IR antagonists such as CSF-IR antagonist antibodies including RG7155 (WO11/70024, WO11/107553, WO11/131407, WO13/87699, WO13/119716, WO13/132044) or FPA-008 (WO11/140249; WO13169264; WO14/036357).

In another aspect, the disclosed anti-CD40 antibody can be combined with one or more of: agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), or reverse/prevent T cell anergy or exhaustion), and agents that trigger innate immune activation and/or inflammation at tumor sites.

In one aspect, the immuno-oncology agent is a CTLA-4 antagonist, such as an antagonistic CTLA-4 antibody. Suitable CTLA-4 antibodies include, for example, ipilimumab (e.g., YERVOY®; Bristol Myers Squibb) or tremelimumab. In another aspect, the immuno-oncology agent is a PD-L1 antagonist, such as an antagonistic PD-L1 antibody. Suitable PD-L1 antibodies include, for example, atezolizumab (MPDL3280A; WO2010/077634) (e.g., TECENTRIQ®; Roche/Genentech), durvalumab (MEDI4736), BMS-936559 (WO2007/005874), and MSB0010718C (WO2013/79174). In another aspect, the immuno-oncology agent is a LAG-3 antagonist, such as an antagonistic LAG-3 antibody. Suitable LAG-3 antibodies include, for example, BMS-986016 (WO10/19570, WO14/08218), or IMP-731 or IMP-321 (WO08/132601, WO09/44273). In another aspect, the immuno-oncology agent is a CD137 (4-1BB) agonist, such as an agonistic CD137 antibody. Suitable CD137 antibodies include, for example, urelumab and PF-05082566 (WO12/32433). In another aspect, the immuno-oncology agent is a GITR agonist, such as an agonistic GITR antibody. Suitable GITR antibodies include, for example, BMS-986153, BMS-986156, TRX-518 (WO06/105021, WO09/009116) and MK-4166 (WO11/028683). In another aspect, the immuno-oncology agent is an OX40 agonist, such as an agonistic OX40 antibody. Suitable OX40 antibodies include, for example, MEDI-6383 or MEDI-6469. In another aspect, the immuno-oncology agent is an OX40L antagonist, such as an antagonistic OX40 antibody. Suitable OX40L antagonists include, for example, RG-7888 (WO06/029879). In another aspect, the immuno-oncology agent is a CD27 agonist, such as an agonistic CD27 antibody. Suitable CD27 antibodies include, for example, varlilumab. In another aspect, the immuno-oncology agent is MGA271 (to B7H3) (WO11/109400). In still another embodiment, combination of anti-CD40 antibodies according to this disclosure with an agent directed at Trop-2, e.g., the antibody drug conjugate, sacituzumab govitecan-hziy, is contemplated. In yet another embodiment, combination of the anti-CD40 antibodies described herein with an agent that inhibits the CD47-SIRPα pathway is contemplated. An example of an anti-CD47 antibody is magrolimab.

In some embodiments, a combination is an antibody of the present disclosure with a second antibody directed at a surface antigen preferentially expressed on the cancer cells relative to control normal tissue. Some examples of antibodies that can be administered in combination therapy with antibodies of the present disclosure for treatment of cancer include Herceptin® (trastuzumab) against the HER2 antigen, Avastin® (bevacizumab) against VEGF, or antibodies to the EGF receptor, such as (Erbitux®, cetuximab), and Vectibix® (panitumumab). Other agents that can be administered include antibodies or other inhibitors of any of PD-1, PD-L1, CTLA-4, 4-1BB, BTLA, PVRIG, VISTA, TIM-3 and LAG-3; or other downstream signaling inhibitors, e.g., mTOR and GSK3ß inhibitors; and cytokines, e.g., interferon-γ, IL-2, and IL-15. Some specific examples of additional agents include: ipilimumab, pazopanib, sunitinib, dasatinib, pembrolizumab, INCR024360, dabrafenib, trametinib, atezolizumab (MPDL3280A), erlotinib (e.g., TARCEVA®), cobimetinib, nivolumab, and zimberelimab. The choice of a second antibody or other agent for combination therapy depends on the cancer being treated. Optionally, the cancer is tested for expression or preferential expression of an antigen to guide selection of an appropriate antibody. In some embodiments, the isotype of the second antibody is human IgG1 to promote effector functions, such as ADCC, CDC and phagocytosis.

The present disclosure encompasses pharmaceutically acceptable salts, acids or derivatives of any of the above.

### V. Kits

Antibodies against CD40 can be combined with any of the second antibodies or agents described for use in co-therapies as components of a kit. The disclosure disclosed herein provides one or more kits containing one or more of the antibodies disclosed herein as well as one or more pharmaceutically acceptable excipients or carriers (such as, without limitation, phosphate buffered saline solutions, water, sterile water, polyethylene glycol, polyvinyl pyrrolidone, lecithin, arachis oil, sesame oil, emulsions such as oil/water emulsions or water/oil emulsions, microemulsions, nanocarriers and various types of wetting agents). Additives such as alcohols, oils, glycols, preservatives, flavoring agents, coloring agents, suspending agents, and the like can also be included in the kits of the present disclosure along with the carrier, diluent, or excipient. In one embodiment, a pharmaceutically acceptable carrier appropriate for use in the antibody compositions disclosed herein is sterile, pathogen free, and/or otherwise safe for administration to a subject without risk of associated infection and other undue adverse side effects. In a kit, the respective agents can be provided in separate vials with instructions for combination followed by administration or instructions for separate administration. The kit can also include written instructions for proper handling and storage of any of the anti-CD40 antibodies disclosed herein.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All patent filings, websites, other publications, accession numbers and the like cited above or below are incorporated by reference in their entirety for all purposes to the same extent as if each individual item were specifically and individually indicated to e so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the disclosure can be used in combination with any other unless specifically indicated otherwise.

Although the present disclosure has been described in some detail by way of illustration and example for purposes of clarity and understanding, it will be apparent that certain changes and modifications can be practiced within the scope of the appended claims.

### EXAMPLES

These examples are provided for illustrative purposes only and not to limit the scope of the claims provided herein.

### Example 1. Clinical Trial Phase 2 Study Design

Results from a Phase 1b trial evaluating gemcitabine and nab-paclitaxel with or without sotigalimab demonstrated promising clinical activity in patients with untreated metastatic pancreatic ductal adenocarcinoma (mPDAC) (O'Hara et al. Lancet Oncol. 2021; 22(1): 118-131). The Phase 1b trial was a dose-ranging study to assess safety and clinical activity and to determine the recommended Phase 2 dose of sotigalimab in combination with gemcitabine (Gem) and nab-paclitaxel (NP) with or without nivolumab. Presented herein are results from the follow-on, randomized phase 2 trial (NCT03214250) evaluating gemcitabine and nab-paclitaxel with or without sotigalimab.

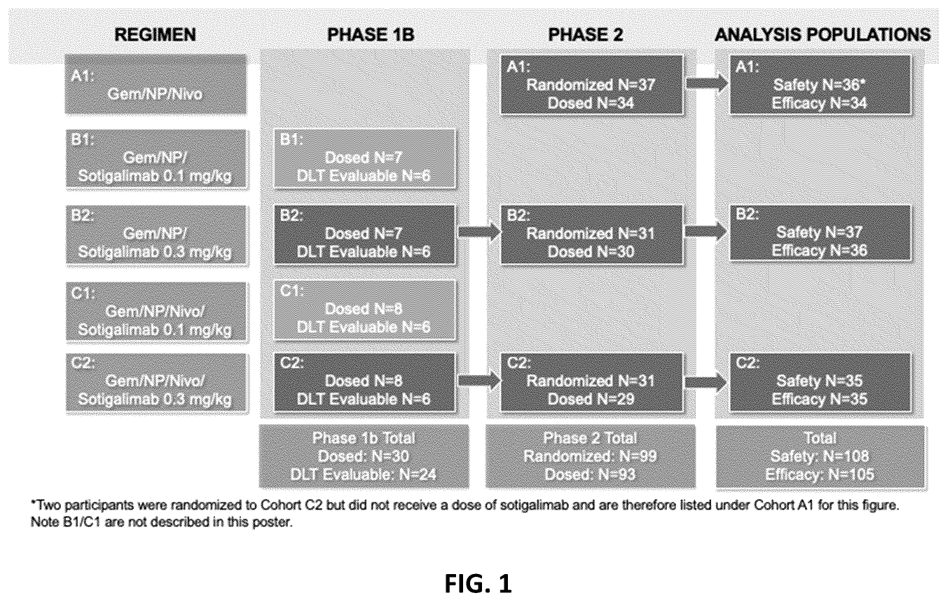

The first 12 participants were randomized 4:1:1 to A1 (Gem+NP+Nivolumab), B2 (Gem+NP+Sotigalimab 0.3 mg/kg), or C2 (Gem+NP+Nivolumab+Sotigalimab 0.3 mg/kg). The remaining participants were randomized in a 1:1:1 allocation. The 12 dose-limiting toxicity (DLT)-evaluable participants from Phase 1b (6 in B2 and 6 in C2) were included in Phase 2 efficacy analyses. (FIG. 1)

Primary endpoint was 1-year overall survival (OS) rate compared with a 35% historical rate for Gem+NP (Von Hoff et al. N Engl J Med. 2013; 369(18): 1691-1703). Secondary endpoint was safety (adverse events [AEs], treatment-related adverse events [TRAEs]), objective response rate (ORR), disease control rate (DCR), progression-free survival (PFS), and duration of response (DOR). Exploratory endpoint was immune pharmacodynamics, associations between immune biomarkers and clinical outcomes, and baseline and on-treatment microbiome profiles.

Participants were eligible for enrollment if they had histological or cytological diagnosis of metastatic pancreatic adenocarcinoma and Eastern Cooperative Oncology Group (ECOG) 0, or 1; no prior treatment for metastatic disease was permitted, nor was prior CD40, PD-1, PD-L1, CTLA-4 treatment in any setting. The enrollment period for Phase 2 was from Aug. 30, 2018 to Jun. 10, 2019.

Dosing schedule was on day 1, day 8, and day 15 chemotherapy for each 28-day cycle. Gemcitabine (1000 mg/m²)+nab-paclitaxel (125 mg/m²) were administered. Both were starting doses. For cohorts A1 and C2, on day 1 and day 15, nivolumab 240 mg was administered.

Tumor biopsies were collected at screening and cycle 2 day 4 (cohorts with sotigalimab) or day 8 (cohorts without sotigalimab) and end of treatment (optional). Baseline (cycle 1 day 1 or at screening) and on-treatment blood, tumor tissue, and stool samples were collected and analyzed for tumor and immune biomarkers using a variety of technologies known in the art. Planned enrollment of 35 patients/arm provided 81% power for testing the alternative of 58% OS rate vs. 35%, using a 1-sided, 1-sample Z test with 5% type I error. Trial was not powered for cross-arm comparison.

### Example 2. Clinical Trial Phase 2 Study Population

All participants had a minimum follow-up of 15 months at the time of the data snapshot presented as Table. 1 (March 2021).

TABLE 1
Demographics and Baseline Characteristics (Safety Population)

| | Cohort A1 | | Cohort B2 | | Cohort C2 | | Total | |
|---|---|---|---|---|---|---|---|---|
| Demographics | (N = 34) | | (N = 37) | | (N = 37) | | (N = 108) | |
| Age (yr) | | | | | | | | |
| Median (IQR) | 62.5 | (54-67) | 61.0 | (55-69) | 62.0 | (57-69) | 62.0 | (65-68.5) |
| Sex | | | | | | | | |
| Male | 20 | (58.8%) | 24 | (64.9%) | 20 | (54.1%) | 64 | (59.3%) |
| Race or ethnic group | | | | | | | | |
| Asian | 3 | (8.8%) | 4 | (10.8%) | 1 | (2.7%) | 8 | (7.4%) |
| Black | 0 | | 3 | (8.1%) | 2 | (5.4%) | 5 | (4.6%) |
| Other | 2 | (5.9%) | 1 | (2.7%) | 2 | (5.4%) | 5 | (4.6%) |
| White | 29 | (85.3%) | 29 | (78.4%) | 32 | (86.5%) | 90 | (83.3%) |
| Hispanic or Latino | 1 | (2.9%) | 1 | (2.7%) | 1 | (2.7%) | 3 | (2.8%) |
| ECOG Performance | | | | | | | | |
| Score at Screening | | | | | | | | |
| 0 | 15 | (44.1%) | 20 | (54.1%) | 17 | (45.9%) | 52 | (48.1%) |
| 1 | 19 | (55.9%) | 17 | (45.9%) | 20 | (54.1%) | 56 | (51.9%) |
| Pancreatic Tumor Location | | | | | | | | |
| Body | 12 | (35.3%) | 10 | (27.0%) | 10 | (27.0%) | 32 | (29.6%) |
| Head | 14 | (41.2%) | 17 | (45.9%) | 20 | (54.1%) | 51 | (47.2%) |
| Tail | 8 | (23.5%) | 10 | (27.0%) | 7 | (18.9%) | 25 | (23.1%) |
| Neutrophil-Lymphocyte | | | | | | | | |
| Ratio (NLR) at Screening | | | | | | | | |
| <5 | 26 | (76.5%) | 23 | (62.2%) | 23 | (62.2%) | 72 | (66.7%) |
| ≥5 | 8 | (23.5%) | 14 | (37.8%) | 14 | (37.8%) | 36 | (33.3%) |
| CA19-9 (U/mL) at Cycle 1, Day 1 | | | | | | | | |
| n | 25 | | 26 | | 31 | | 82 | |
| <100 | 3 | (12.0%) | 4 | (15.4%) | 3 | (9.7%) | 10 | (12.2%) |
| 100-1000 | 4 | (16.0%) | 7 | (26.9%) | 9 | (29.0%) | 20 | (24.4%) |
| ≥1000 | 18 | (72.0%) | 15 | (57.7%) | 19 | (61.3%) | 52 | (63.4%) |
| Number of Evaluable Participants | 23 | | 19 | | 20 | | 62 | |
| KRAS Mutations | | | | | | | | |
| Gly12D | 9 | (39.1%) | 7 | (36.8%) | 6 | (30.0%) | 22 | (35.5%) |
| Gly12V | 7 | (30.4%) | 5 | (26.3%) | 5 | (25.0%) | 17 | (27.4%) |
| Gly12R | 2 | (8.7%) | 3 | (15.8%) | 1 | (5.0%) | 6 | (9.7%) |
| Other | 1 | (4.4%) | 3 | (15.8%) | 1 | (5.0%) | 5 | (8.1%) |

| | | | | | | |
|---|---|---|---|---|---|---|
| MSI-H | 1 | (4.4%) | 0 | 0 | 1 | (1.6%) |

Note:
Cohort A1: Gem + NP + Nivolumab, Cohort B2: Gem + NP + Soligalimab. Cohort C2: Gem + NP + Nivolumab + Soligalimab

Baseline characteristics were generally balanced across arms, inclusive of tumor burden, presence of liver metastases (25 [73.5%], 28 [75.7%], 27 [73.0%] for A1, B2, and C2, respectively) and stage at initial diagnosis (stage 1-3 versus stage 4 [stage 4: 27 (79.4%), 28 (75.5%), 27 (73.0%) for A1, B2, and C2, respectively]) (Table 1).

### Example 3. Efficacy

Median time on treatment was 5.2, 5.1, and 4.7 months for cohorts A1, B2, and C2, respectively. One year OS rate was 57.3% (1-sided p=0.007, 95% lower CI bound=41%) for A1, 48.1% (p=0.062, 95% lower CI bound=34%) for B2, and 41.3% (p=0.236, 95% lower CI bound=27%) for C2 vs. 35% historical rate. The single MSI-H patient in A1 had an OS of 249 days and therefore does not meaningfully impact interpretation of the primary endpoint. Median OS and secondary endpoints are listed in Table 2.

TABLE 2
Overall Survival And Secondary Endpoints for Efficacy Population.

| % (n) [95% CI] | A1 (n = 34) | | B2 (n = 36) | | C2 (n = 35) | |
|---|---|---|---|---|---|---|
| ORR* | 50 (17) | [32-68] | 33 (12) | [19-51] | 31 (11) | [17-49] |
| ORR (confirmed)* | 35 (12) | [20-54] | 33 (12) | [19-51] | 26 (9) | [13-43] |
| DCR | 74 (25) | [56-87] | 78 (28) | [61-90] | 69 (24) | [51-83] |
| Median DOR, mos | 7.3 | [2.1-NE] | 5.5 | [3.7-7.9] | 7.9 | [1.9-NE] |
| Median PFS, mos | 6.3 | [5.2-8.8] | 7.2 | [5.3-9.2] | 6.7 | [4.1-9.8] |
| Median OS, mos | 16.7 | [9.8-18.4] | 11.4 | [7.2-20.1] | 10.1 | [7.9-13.2] |
| 1-year OS, % [p] | 57.3 | [0.007] | 48.1 | [0.062] | 41.3 | [0.236] |

*1 CR observed in A1; NE = not estimable.

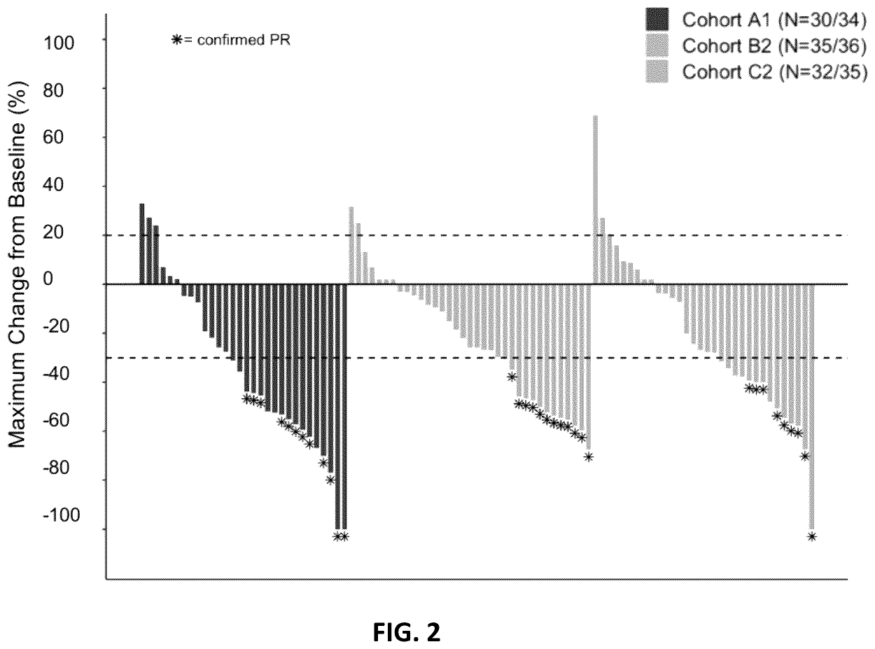

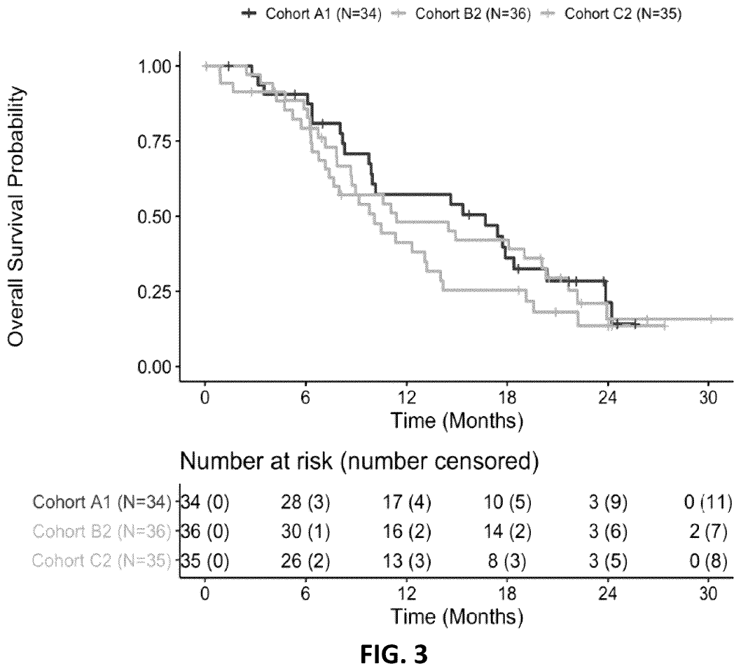

FIG. 2 shows the percentage changes in the sum of target lesions, and FIG. 3 shows OS.

### Example 3. Safety

Rates of treatment-related adverse events (TRAEs) were overall similar and consistent across cohorts and with Phase 1b portion of the study. Eight participants (7%) experienced an adverse event (AE) leading to treatment discontinuation, of which seven were from A1 (peripheral neuropathy, myocarditis, pneumonitis, thrombotic microangiography (2), and hyperbilirubinemia, one from B2 (pneumonitis), one from C2 (pyrexia). 98.1% of participants experienced a TRAE, with at least one having a grade 3 or 4 event (66.7%, 86.5%, 80.0% for A1, B2, and C2, respectively). The top 5 TRAEs occurring in 10% or more of participants by preferred term are shown in Table 3.

TABLE 3
Most Frequent TRAEs by Medical Dictionary for Regulatory Activities (MedDRA) Preferred Term.

| | Cohort A1 | | | Cohort B2 | | | Cohort C2 | | |
|---|---|---|---|---|---|---|---|---|---|
| | (N = 36) | | | (N = 37) | | | (N = 35) | | |
| MedDRA Preferred Term | Any Grade | | Grade 3-4 | Any Grade | Grade 3-4 | | Any Grade | Grade 3-4 | |
| Nausea | 25 | (69.4%) | 0 | 32 (86.5%) | 0 | | 28 (80.0%) | 0 | |
| Fatigue | 25 | (69.4%) | 9 (25.0%) | 27 (73.0%) | 5 | (13.5%) | 27 (77.1%) | 5 | (14.3%) |
| Pyrexia | 11 | (30.6%) | 0 | 28 (75.7%) | 1 | (2.7%) | 24 (68.6%) | 1 | (2.9%) |
| Aspartate aminotransferase increased | 18 | (50.0%) | 7 (19.4%) | 24 (64.9%) | 14 | (37.8%) | 20 (57.1%) | 9 | (25.7%) |

| | | | | | | | | |
|---|---|---|---|---|---|---|---|---|
| Chills | 3 | (8.3%) | 0 | 30 (81.1%) | 3 | (8.1%) | 27 (77.1%) | 0 |

Note:
Cohort A1: Gem + NP + Nivolumab, Cohort B2: Gem + NP + Sotigalimab, Cohort C2: Gem + NP + Nivolumab + Sotigalimab

39 participants (36%) experienced a serious TRAE (13, 15, and 11 in A1, B2, and C2, respectively) and 2 participants died due to TRAEs; 1 each in B2 (acute hepatic failure possibly related to all study drugs) and C2 (intracranial hemorrhage possibly related to all study drugs). Cytokine release syndrome occurred in 0, 9 (24.3%), and 12 (34.3%) participants in A1, B2, and C2, respectively, with 0, 3 (8.1%), and 2 (5.7%) participants at grade 3-4 in A1, B2, and C2, respectively.

### Example 4. Pharmacodynamic Effects

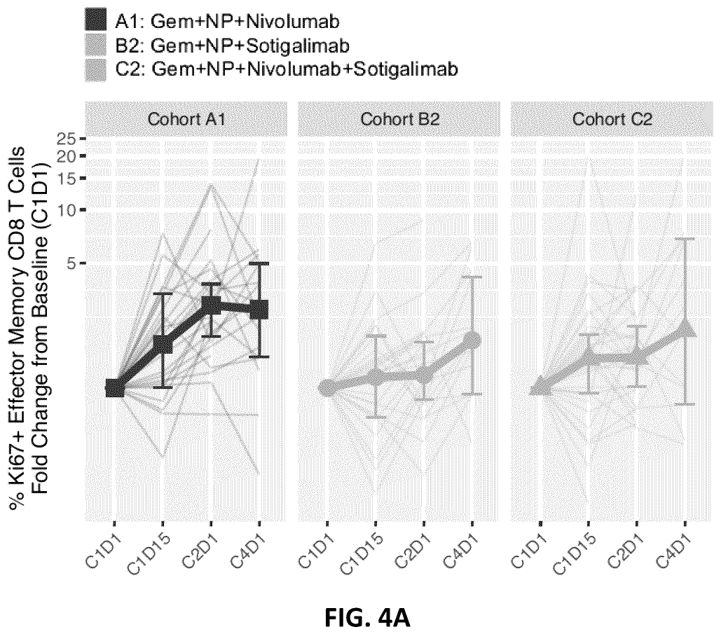

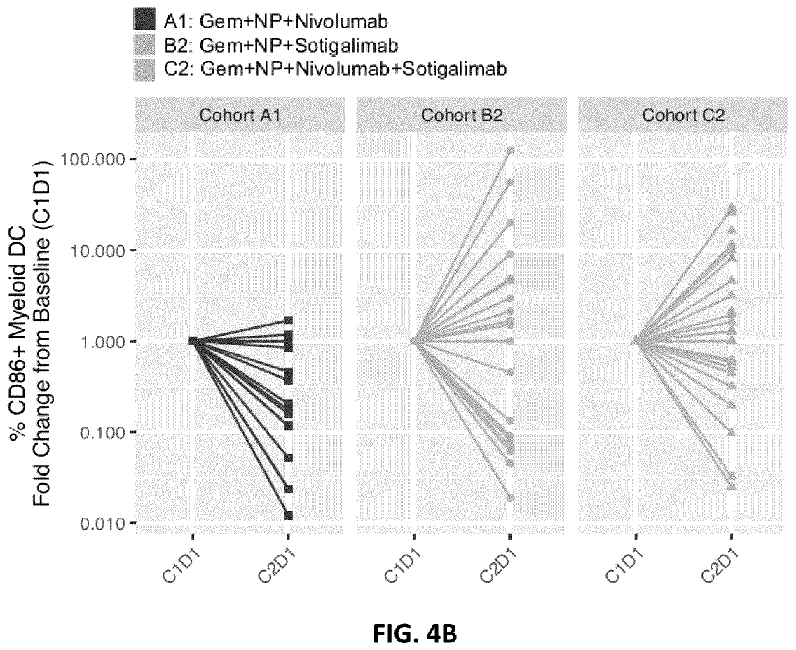

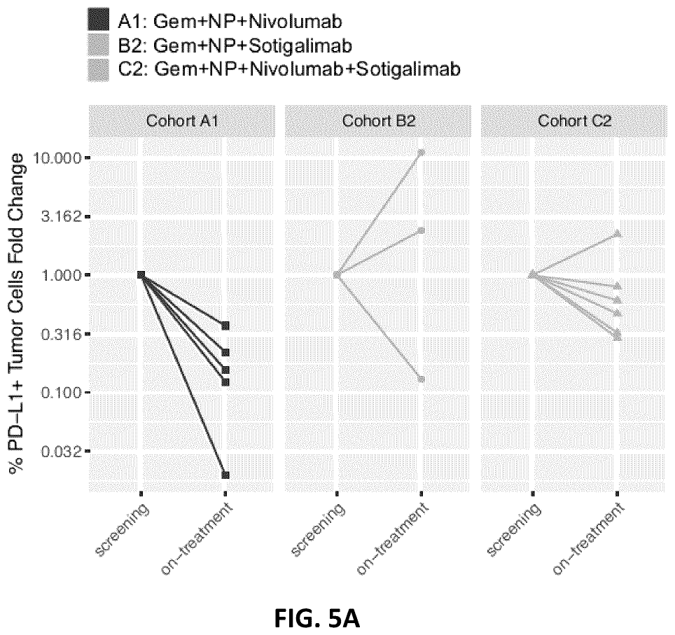

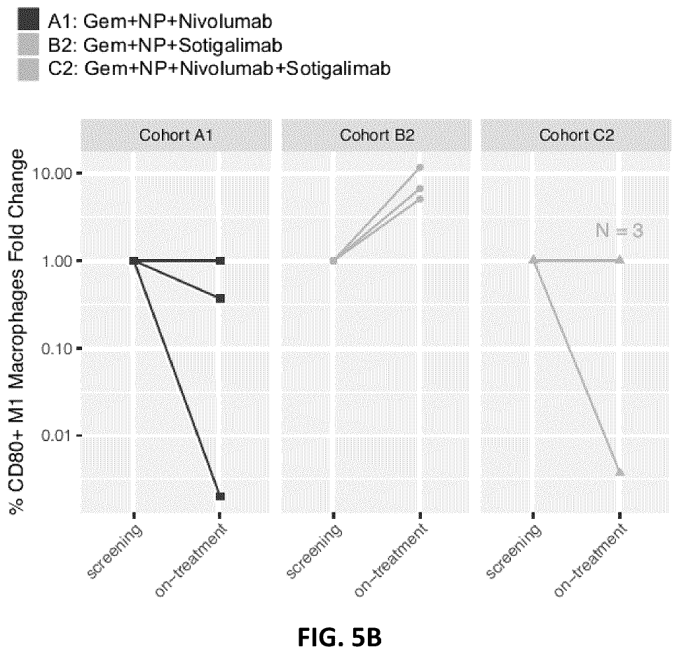

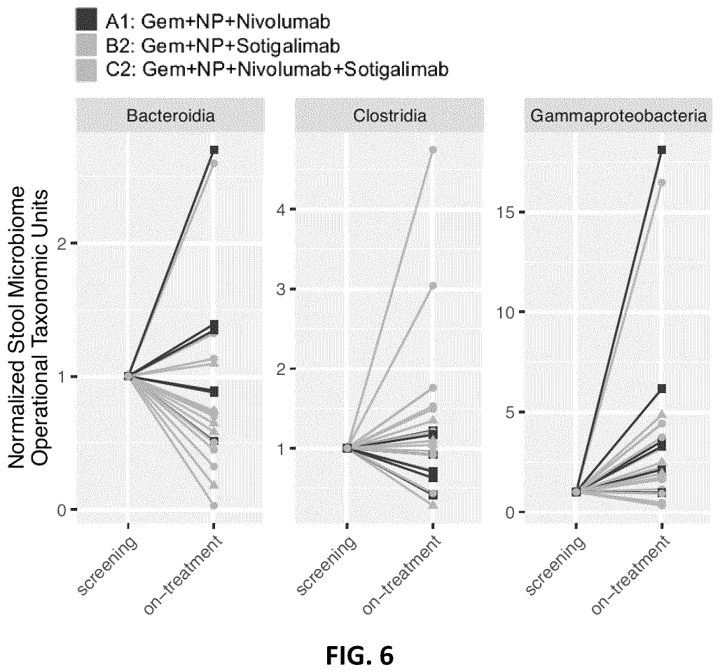

Immune pharmacodynamic effects consistent with the immunotherapy mechanism of action were observed with the treatment in blood, tumor, and stool (FIGS. 4A, 4B, 5A, and 5B). All 3 cohorts showed an increase in activated effector memory (EM) T cells (Ki67+CD8+ cells (FIG. 4A)/CD4+EM cells (data not shown)), with nivolumab+chemotherapy (cohort A1) inducing the most pronounced effect. An increase in activated myeloid dendritic cells (CD86⁺+mDC) occurred in the majority of participants in cohort B2 (sotigalimab+chemotherapy) and frequently in cohort C2 (nivolumab+sotigalimab+chemotherapy) as an expected pharmacodynamic effect of sotigalimab, whereas nivolumab+chemotherapy (A1) treatment predominantly resulted in a decrease (FIG. 4B). A decrease in the percentage of tumor cells expressing PD-L1 was observed in response to treatment with nivolumab (A1, n=5; and C2, n=6) in most tumors, whereas sotigalimab+chemotherapy (B2, n=3) showed mixed changes in PD-L1 expression (FIG. 5A). Sotigalimab+chemotherapy (B2, n=2) treatment increased in tumoral CD80⁺ M1 macrophages, whereas nivolumab-containing treatments decreased (A1, n=2; and C2, n=1) (FIG. 5B). Nivolumab+chemotherapy (A1) treatment increased bacteroidia and decreased clostridia, whereas sotigalimab+chemotherapy (B2) showed the opposite effect. All 3 treatment arms displayed increases in gammaproteobacteria consistent with a chemotherapy effect (FIG. 6).

### Example 5. Baseline Immune and Tumor Biomarkers Associated with Clinical Outcomes

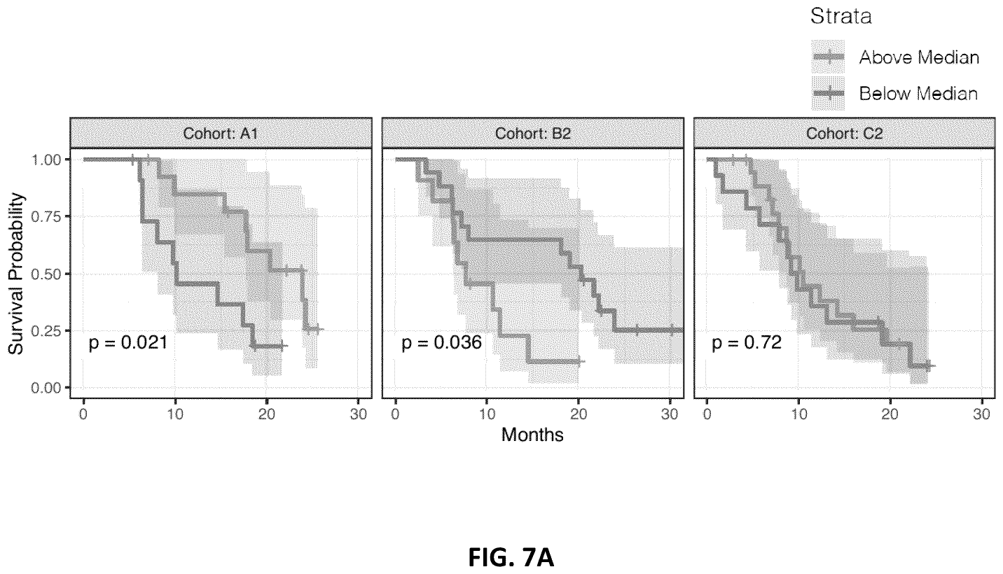

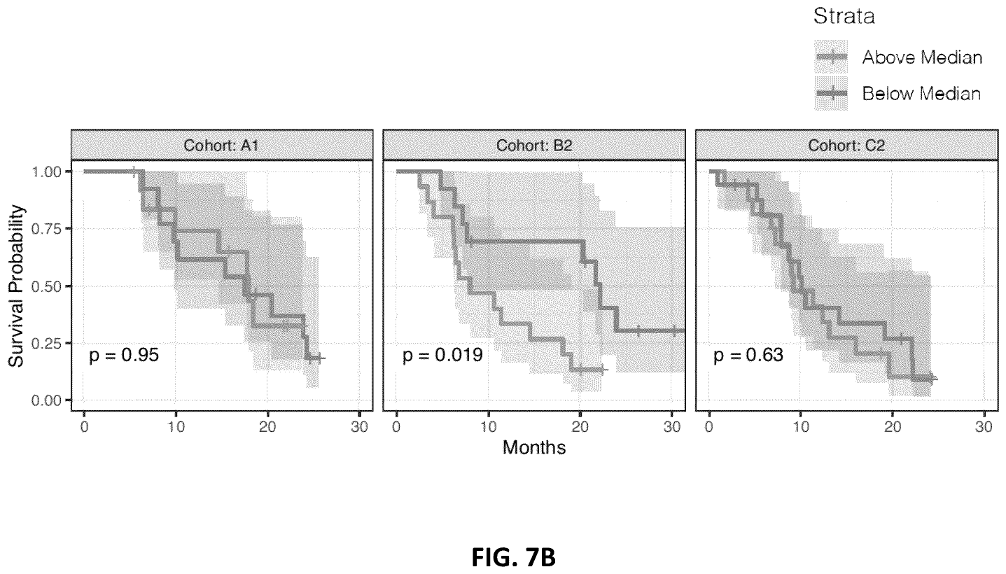

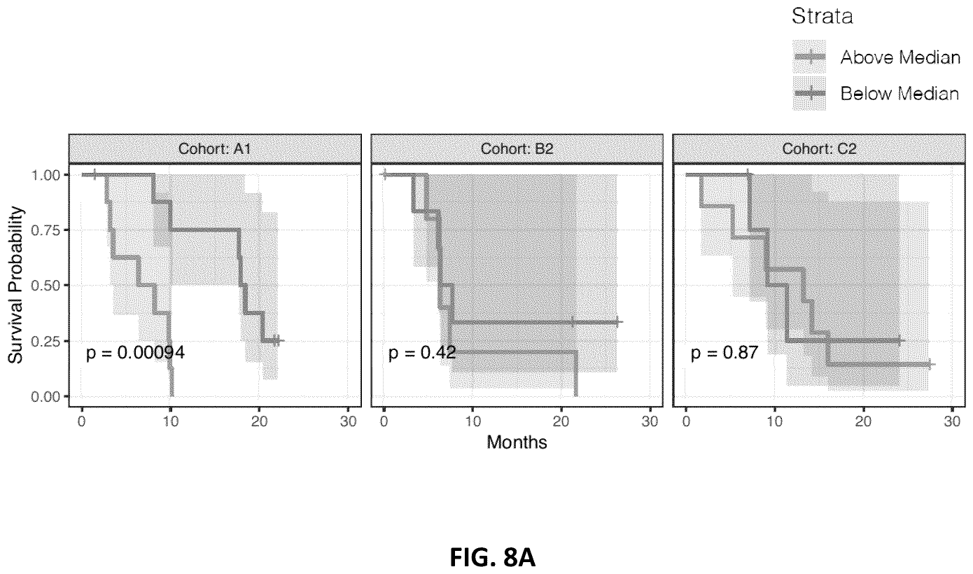

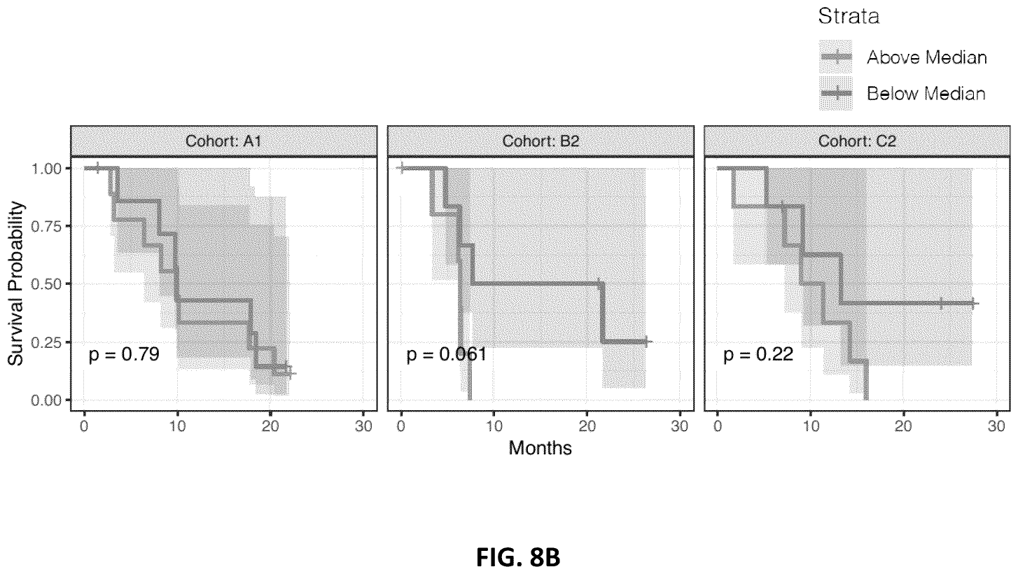

Baseline blood, tumor, and stool biomarkers defined different subsets of PDAC participants that were associated with improved overall survival with nivolumab+chemotherapy and/or sotigalimab+chemotherapy treatment but not the immunotherapy combination. Higher baseline levels of CXCR5⁺ EM CD8⁺ T cells (FIG. 7A) were associated with improved survival in response to nivolumab+chemotherapy (A1) treatment, whereas lower baseline levels were associated with improved survival with sotigalimab+chemotherapy (B2) but not the nivolumab+sotigalimab combination (C2). Lower baseline levels of exhausted (CD244⁺) EM CD4⁺ T cells were associated with improved survival in response to sotigalimab+chemotherapy (B2) treatment, but no difference in survival outcomes was observed in the cohorts containing nivolumab treatment (FIG. 7B). Lower baseline levels of inflammatory gene signature (TNFα) were associated with improved survival in response to nivolumab+chemotherapy (A1, n=17) treatment, but no difference in survival outcomes was observed in the sotigalimab-containing arms (B2, n=12; C2, n=12) (FIG. 8A). Lower level of MYC gene signatures (FIG. 8B) were associated with improved survival in response to sotigalimab+chemotherapy (B2, n=12) treatment, but no difference in survival outcomes was observed in the nivolumab-containing arms (A1, n=17; C2, n=12).

The primary endpoint of 1-year OS rate >35% was met in A1 (nivolumab+chemotherapy) in contrast with previously reported data in this setting (O'Hara et al. Lancet Oncol. 2021; 22(1): 118-131). The primary endpoint was not met in B2 or C2, although moderate clinical activity was observed in B2 (sotigalimab+chemotherapy). Safety profiles of the IO+chemotherapy treatments across the 3 cohorts were manageable and consistent with previously reported Phase 1b data. Comprehensive multi-omic analyses of pre- and on-treatment blood, tissue, and stool samples revealed expected pharmacodynamic effects and immune activation in A1 and B2. Moreover, biomarker signature that associate with patient subsets with clinical benefit in response to nivolumab+chemotherapy (A1) do not overlap with signatures associated with benefit to sotigalimab+chemotherapy (B2). Such signatures were associated with use of immunotherapy but not chemotherapy. The combination of sotigalimab, nivolumab, and chemotherapy treatment (C2) exhibited mixed pharmacodynamic effects and did not have a clear biomarker subset that showed benefit, raising the potential hypothesis of IO-10 drug antagonism in this setting. Given observed clinical activity and hypothesis-generating biomarker results, further exploration and prospective testing of baseline biomarkers is warranted to improve clinical precision of IO+chemotherapy in PDAC, and a platform study (REVOLUTION, NCT04787991), has been initiated to build on these data.

### Example 6. Identification of Circulating Immune Cells and Hallmark Gene Signature Analysis

### Whole Exome and RNA Sequencing of Bulk Tumor Tissue

For each patient, a single paired formalin fixed, paraffin embedded or fresh frozen tumor and normal peripheral blood mononuclear cell (PBMC) sample was collected and profiled using the ImmunoID NEXT platform (Personalis, Inc) for whole exome and transcriptome analysis. The resulting data were used for gene expression quantification. Whole-transcriptome sequencing results were aligned using STAR and normalized expression values in transcripts per million (TPM) were calculated using Personalis' ImmunoID NEXT tool, Expressionist.

### Immune Profiling of Patient PBMCs Using the X50 Platform

Peripheral blood was collected via venipuncture into EDTA vacutainer tubes and PBMC samples were processed at baseline, C1D1 (before treatment). A multiplex flow panel designed to evaluate T cell phenotype & function was utilized. All samples were thawed, stained for viability and antibodies, and run under uniform protocols at the University of Pennsylvania.

### Whole Exome and RNA Sequencing of Bulk Tumor Tissue

For each patient, a single paired formalin fixed, paraffin embedded or fresh frozen tumor and normal peripheral blood mononuclear cell (PBMC) sample was collected and profiled using the ImmunoID NEXT platform (Personalis, Inc) for whole exome and transcriptome analysis. The resulting data were used for gene expression quantification. Whole-transcriptome sequencing results were aligned using STAR and normalized expression values in transcripts per million (TPM) were calculated using Personalis' ImmunoID NeXT tool, Expressionist.

### Immune Profiling of Patient PBMCs Using the X50 Platform

Peripheral blood was collected via venipuncture into EDTA vacutainer tubes and PBMC samples were processed at baseline, C1D1 (before treatment). A multiplex flow panel designed to evaluate T cell phenotype & function was utilized. All samples were thawed, stained for viability and antibodies, and run under uniform protocols at the University of Pennsylvania.

### Identification of Circulating Immune Cells

Patient peripheral blood mononuclear cells (PBMCs) were identified as live CD45⁺ cells. Patient PBMCs were classified into different immune cell populations based on the presence of surface markers. CD8⁺ T cells were selected from CD45⁺ cells by the presence of CD3 and CD8 surface markers. CD4⁺ T cells were selected from CD45⁺ cells by the presence of CD3 and CD4 surface markers. CD8⁺ T cells were further subdivided into numerous T cell subsets, such as Effector Memory Type 1 (EM1) cells. EM1 T cells are classically defined as CD45RA⁻CD27⁺. This cell population was further categorized by CXCR5 expression into a CXCR5⁺ population. The ratio of cell counts in this CXCR5⁺ population to the total EM1 T cell population count was shown to be associated with overall survival. CD4⁺ T cells were further subdivided into numerous T cell subsets, such as Effector Memory Type 3 (EM3) cells. EM3 T cells are classically defined as CD45RA⁻CD27⁻. This cell population was further categorized by CD244 expression into a CD244⁺ population. The ratio of cell counts in this CD244⁺ population to the total EM3 T cell population count was shown to be associated with overall survival.

For overall survival analysis, patients were stratified based on the percentage of CXCR5 expression on EM1 CD8⁺ T cells, where “high” vs “low” frequencies were defined by the median ratio across all subjects in the cohort. Overall survival analysis indicated lower ratios of CXCR5⁺ EM1 CD8⁺ to total EM1 CD8⁺ were associated with longer survival rates in patients treated with nivolumab in combination with gemcitabine+nab-Paclitaxel and shorter survival rates in patients treated with sotigalimab in combination with gemcitabine+nab-Paclitaxel. (FIGS. 4-8).

### Hallmark Gene Signature Analyses

Hallmark gene signatures are publicly accessible through the Molecular Signatures Database (V7.4) for gene set enrichment analysis (GSEA). The gene sets below include genes belonging to the following gene families: (1) tumor suppressors; (2) oncogenes; (3) translocated cancer genes; (4) protein kinases; (5) cell differentiation markers; (6) homeodomain proteins; (7) transcription factors; and (8) cytokine and growth factors.

The MYC hallmark geneset is comprised of a total of 200 genes known to be regulated by MYC. A total “score” was calculated for MYC by averaging the log normalized expression values for each gene in the geneset and determining the median. For survival analysis, patients were stratified based on the value of this MYC gene signature, where “high” vs “low” was defined by the median signature value across all patients in all cohorts. The individual gene list for the MYC hallmark gene signature is listed on Table 4.

TABLE 4
The Genes of the MYC Hallmark Gene Signature.

ABCE1
ACP1
AIMP2
AP3S1
APEX1
BUB3
C1QBP
CAD
CANX
CBX3
CCNA2
CCT2
CCT3
CCT4
CCT5
CCT7
CDC20
CDC45
CDK2
CDK4
CLNS1A
CNBP
COPS5
COX5A
CSTF2
CTPS1
CUL1
CYC1
DDX18
DDX21
DEK
DHX15
DUT
EEF1B2
EIF1AX
EIF2S1
EIF2S2
EIF3B
EIF3D
EIF3J
EIF4A1
EIF4E
EIF4G2
EIF4H
EPRS1
ERH
ETF1
EXOSC7
FAM120A
FBL
G3BP1
GLO1
GNL3
GOT2
GSPT1
H2AZ1
HDAC2
HDDC2
HDGF
HNRNPA1
HNRNPA2B1
HNRNPA3
HNRNPC
HNRNPD
HNRNPR
HNRNPU
HPRT1
HSP90AB1
HSPD1
HSPE1
IARS1
IFRD1
ILF2
IMPDH2
KARS1
KPNA2
KPNB1
LDHA
LSM2
LSM7
MAD2L1
MCM2
MCM4
MCM5
MCM6
MCM7
MRPL23
MRPL9
MRPS18B
MYC
NAP1L1
NCBP1
NCBP2
NDUFAB1
NHP2
NME1
NOLC1
NOP16
NOP56
NPM1
ODC1
ORC2
PA2G4
PABPC1
PABPC4
PCBP1
PCNA
PGK1
PHB
PHB2
POLD2
POLE3
PPIA
PPM1G
PRDX3
PRDX4
PRPF31
PRPS2
PSMA1
PSMA2
PSMA4
PSMA6
PSMA7
PSMB2
PSMB3
PSMC4
PSMC6
PSMD1
PSMD14
PSMD3
PSMD7
PSMD8
PTGES3
PWP1
RACK1
RAD23B
RAN
RANBP1
RFC4
RNPS1
RPL14
RPL18
RPL22
RPL34
RPL6
RPLP0
RPS10
RPS2
RPS3
RPS5
RPS6
RRM1
RRP9
RSL1D1
RUVBL2
SERBP1
SET
SF3A1
SF3B3
SLC25A3
SMARCC1
SNRPA
SNRPA1
SNRPB2
SNRPD1
SNRPD2
SNRPD3
SNRPG
SRM
SRPK1
SRSF1
SRSF2
SRSF3
SRSF7
SSB
SSBP1
STARD7
SYNCRIP
TARDBP
TCP1
TFDP1
TOMM70
TRA2B
TRIM28
TUFM
TXNL4A
TYMS
U2AF1
UBA2
UBE2E1
UBE2L3
USP1
VBP1
VDAC1
VDAC3
XPO1
XPOT
XRCC6
YWHAE
YWHAQ

### Methods for Examples 7-15

### Study Design and Safety Monitoring

The following examples represent a further analysis of data obtained in Examples 1-6. In this Phase Ib/II study, patients ≥18 years of age with mPDAC were enrolled from 7 academic hospitals in the US which are part of the Parker Institute for Cancer Immunotherapy pancreas cancer consortium. Prior treatment for metastatic disease was not allowed, though prior adjuvant and neoadjuvant chemo/radiotherapy was allowed if completed >4 months prior to enrollment. Patients were required to have archival or fresh tumor specimens available before treatment or be able to undergo a biopsy to acquire tissue. Additional key eligibility criteria included Eastern Cooperative Oncology Group (ECOG) performance status score of 0-1, adequate organ function, and the presence of at least one measurable lesion per Response Evaluation Criteria in Solid Tumors version 1.1 (RECIST v. 1.1). Patients were excluded if they had previous exposure to agonistic CD40, anti-PD-1, anti-PD-L1 monoclonal antibodies, or any other immunomodulatory anticancer agent. Patients were also excluded if they had ongoing or recent autoimmune disease requiring systemic immunosuppressive therapy, had undergone solid-organ transplantation, or had a concurrent cancer, unless indolent or not considered to be life-threatening (e.g., basal-cell carcinoma).

The Phase Ib trial was an open-label, multicenter, four-cohort, dose ranging study that aimed to identify the recommended phase 2 dose (RP2D) of anti-CD40 sotigalimab (sotiga) in combination with chemo (gemcitabine [gem] and nab-Paclitaxel [NP]), with or without anti-PD1 nivolumab (nivo)¹³. The Phase II trial was a randomized, open-label, multicenter, three-arm, study of chemo combined with nivo, sotiga or both immune modulating agents. A RP2D of 0.3 mg/kg sotiga was defined during the Phase Ib portion of the study by a Data Review Team (DRT) comprised of investigators and sponsor clinical staff. During Phase II, the DRT met to review all safety data for each study arm on a quarterly basis. A Bayesian termination rule was employed to monitor toxicity and determine whether enrollment or dosing in a study arm(s) needed to be halted.

The protocol and all amendments were approved by the lead Institutional Review Board at the University of Pennsylvania and were accepted at all participating sites. The study was conducted in accordance with the principles of the Declaration of Helsinki and the International Conference on Harmonisation Good Clinical Practice guidelines. All patients provided written informed consent before enrollment.

### Randomization and Blinding

The Phase II trial was open label with no blinding. Patients were randomly assigned to one of three arms: nivo/chemo, sotiga/chemo, or sotiga/nivo/chemo. Twelve dose limiting toxicity (DLT)-evaluable patients (6 each on sotiga/chemo and sotiga/nivo/chemo) from Phase Ib were included in analyses of Phase II efficacy (see Statistical Analysis section for details on analysis population definitions). To achieve balance in the total number of patients enrolled in each arm, the first 12 patients enrolled in Phase II were randomly allocated in a 4:1:1 ratio to nivo/chemo, sotiga/chemo or sotiga/nivo/chemo, respectively (because nivo/chemo did not accrue patients in Phase Ib, more patients needed to be enrolled in that arm). The remaining patients were randomly allocated in a 1:1:1 ratio. Randomization was managed by the Parker Institute for Cancer Immunotherapy using an interactive voice/web response system (IxRS). A permuted block design, without stratification by baseline patient or tumor characteristics, was used for randomization. Patients who were randomized but did not receive any study drug were replaced via randomization of additional patients.

### Procedures

For each 28-day cycle, gem/NP at 1,000 and 125 mg/m², respectively, were administered intravenously (iv) on days 1, 8, and 15 for each arm. Nivo was administered at 240 mg iv on days 1 and 15. Sotiga was administered 0.3 mg/kg iv on day 3, two days after chemo. Alternatively, sotiga could be administered on day 10 if not administered on day 3, provided patients received chemo on day 8. Investigators were also given the option to utilize 21-day chemo cycles, in which case the day 15 dose was not administered. Up to 2 dose reductions were permitted for sotiga and gem, and up to 3 dose reductions were permitted for NP for management of toxicity. Nivo was allowed to be withheld, but dose reductions were not permitted. A maximum interruption of 4 weeks was permitted per protocol before study discontinuation was required.

Patients were assessed radiographically every 8 weeks for the first year and every 3 months thereafter, regardless of dose delays. Disease assessments were collected until radiographic progression or initiation of subsequent therapy, whichever occurred first. Patients were subsequently followed for survival. Safety assessments included vital signs, physical examinations, electrocardiograms, and laboratory tests. Adverse events were graded according to the National Cancer Institute Common Terminology Criteria for Adverse Events, version 4.03. Adverse event terms were coded using the Medical Dictionary for Regulatory Activities (MedDRA) version 23.0.

Blood samples for isolation of peripheral blood mononuclear cells (PBMC) were collected longitudinally at participating clinical sites, shipped overnight and processed at a central location (Infinity Biologix, Piscataway, NJ, USA) over a ficoll gradient and cryopreserved. Serum was processed within 2 hours of collection at each site and frozen immediately at −80° C., then batch shipped to a central biorepository. Blood sampling for immune biomarkers occurred during screening, at cycle 1 days 1 and 15, cycles 2-4 day 1 and at treatment discontinuation. If a patient began any new anti-cancer therapy prior to their end of treatment (EOT) visit, samples were not collected. For patients who remained on treatment for at least 1 year, blood was collected at 1 year and every six months thereafter.

Baseline or archival as well as post-treatment tumor specimens were collected for biomarker analyses. Fresh tumor biopsies were immediately snap frozen or formalin fixed and paraffin embedded. Any medically feasible post-treatment tumor samples were accepted; however, the preference was for a sample during cycle 2, after the second dose of sotiga or third dose of nivo depending on the assigned treatment arm. Additional biopsies were allowed for patients who had prolonged stable disease, defined as more than two consecutive disease assessments demonstrating response via RECIST v1.1, as well as at the time of disease progression. Ad hoc biopsy collection was permitted with the approval of the medical monitor.

### Outcomes

The primary endpoint was the 1-year OS rate of each treatment arm, compared to the historical rate of 35% for gem/NP14. Secondary endpoints were progression-free survival (PFS), duration of response (DOR), objective response rate (ORR), disease control rate (DCR), and the incidence of adverse events. Key exploratory endpoints included the evaluation of immune pharmacodynamic (PD) effects and tumor and immune biomarker analyses.

### Statistical Analysis

This study did not include a control arm of gem/NP (chemo). Therefore, the 1-year OS rate for each arm was estimated and compared with a historical value of 35%14. This study was not powered for statistical comparison between arms and no adjustment for multiple comparisons was performed for the clinical endpoints.

The null hypothesis was a 1-year OS rate of 35% and the alternative hypothesis was a 1-year OS rate of 55%. Planned enrollment was 105 patients (35 per arm), which included 12 DLT-evaluable patients from Phase Ib. A sample size of 35 patients per arm provided 81% power to test this hypothesis, using a 1-sample Z test with 5% type I error rate.

Efficacy analyses were conducted on the efficacy population, defined as (1) all patients who were randomized in Phase II and received at least one dose of any study drug and (2) the 12 DLT-evaluable patients (6 on sotiga/chemo and 6 on sotiga/nivo/chemo; defined as experiencing a dose limiting toxicity or receiving at least 2 doses of chemo and one dose of sotiga during cycle 1 who were enrolled in Phase Ib¹³. For efficacy analyses, patients were grouped according to the treatment arm assigned at randomization. Safety analysis was conducted on all Phase Ib (DLT- and non-DLT-evaluable) and Phase II patients who received at least 1 dose of any study drug at the RP2D (defined as the safety population). For safety analyses, patients were grouped according to the study treatment actually received. Two Phase II patients were randomly allocated to sotiga/nivo/chemo but only received doses of chemo and nivo (i.e., sotiga was not received); these patients were grouped as sotiga/nivo/chemo for efficacy analyses and as nivo/chemo for safety and biomarker analyses.

OS was defined as the time from treatment initiation until death due to any cause. Patients who were not reported as having died at the time of analysis were censored at the most recent contact date. OS was estimated by the Kaplan-Meier method for each treatment arm. The 1-year OS rate and corresponding 1-sided, 95% confidence interval (CI) were calculated, to determine whether the lower bound of the CI excludes the assumed historical value of 35%. P-values were calculated using a 1-sided, one-sample Z test against the historical rate of 35%. ORR was defined as the proportion of patients with an investigator-assessed partial response (PR) or complete response (CR) per RECIST version 1.1—confirmation of response was not required; DCR as the proportion of patients with a PR, CR, or stable disease lasting at least 7 weeks as best response; DOR as the time from the first tumor assessment demonstrating response until the date of radiographic disease progression; and PFS as the time from treatment initiation until radiographic disease progression or death (whichever occurred first). Confidence intervals (CI) for ORRs were calculated using the Clopper-Pearson method. The Kaplan-Meier method was used to estimate DOR and PFS and the corresponding CIs. Safety and tolerability were summarized descriptively in terms of adverse events. Statistical analyses were performed using R version 4.1.0 or greater.

### Interim Analysis

Two pre-specified interim analyses (IA) of Phase II clinical data were performed. These IAs were strictly meant to support decision marking for future studies. No adaptations to the study design or conduct were planned based on the interim results and no control of type I error was applied for any of the endpoints at the interim or final analysis. The IAs were performed by the Parker Institute for Cancer Immunotherapy and results were shared with the study investigators and pharmaceutical partners (Apexigen and Bristol Myers Squibb).

The first IA occurred approximately 4 months after the last patient was randomized in Phase II and the second IA occurred approximately 9 months after the last patient was randomized. Both IAs assessed safety and all efficacy endpoints (ORR, DCR, DOR, OS, PFS) for patients enrolled in Phase Ib. In addition, the first IA included Phase II analysis of ORR and DCR and the second IA included Phase II analysis of all efficacy endpoints excluding OS (i.e., ORR, DCR, DOR, PFS). Phase II OS data was not analyzed during any IA.

### Immunophenotyping by Mass Cytometry Time of Flight (CyTOF)

A broad immunophenotyping panel was used on cryopreserved PBMC by CyTOF analysis run under uniform protocols (PMID: 31315057) at Primity Bio (Fremont, CA, USA) in a blinded fashion. Cryopreserved PBMC were thawed in 37° C. prewarmed RPMI-1640 containing 10% FBS and 25 U/mL of benzonase. Samples were washed once more in RPMI-1640 containing 10% FBS and 25 U/mL of benzonase and a third time in 37° C. prewarmed RPMI-1640 containing 10% FBS. Samples were resuspended in 1000 nM of cisplatin for viability discrimination, prepared in PBS containing 0.1% BSA, for 5 minutes at room temperature and then washed with staining buffer. Human BD Fc block was added to the cells for 10 minutes at 4° C. followed by the surface antibody cocktail. The surface staining cocktail was incubated for 30 minutes at 4° C. Samples were washed out of the stain twice with staining buffer. The cells were then resuspended in FoxP3 Transcription Factor 1× Fix/Perm buffer (eBioscience), for 1 hour at room temperature to prepare the cells for intracellular staining. The fixation was then followed by a wash in 1× permeabilization buffer. The intracellular staining cocktail was prepared in the permeabilization buffer and added to the samples and incubated at room temperature for 1 hour. Following the intracellular stain, the samples were washed twice with the permeabilization buffer and once with staining buffer. Prior to acquisition on the CyTOF, samples were resuspended in an iridium (Ir)-intercalating solution for at least 24 hours and stored at 4° C. On the day of acquisition, the samples were washed five times in cell culture grade water (HyClone) and run on the CyTOF Helios instrument (Fluidigm). Details on the CyTOF panel are displayed in Table 5.

TABLE 5

| Mass | Element | Target | Clone | Source | Cat # | Biology | Staining |
|---|---|---|---|---|---|---|---|
| 89 | Y | CD45 | HI30 | Fluidigm | 3089003B | Pan | Surface |
| 113 | In | CD66cd | YTH71.3 | Invitrogen | Custom | Granulocytes | Surface |
| 115 | In | CD7 | M-T701 | BD Biosciences | Custom | T cell subset/NK/monocyte | Surface |
| 140 | Ce | CD86 | IT2.2 | BioLegend | Custom | T cell cos [symbol] /inhibitory | Surface |
| 141 | P [symbol] | CD3 | UCHTI | Fluidigm | 3141019B | Pan T cells | Surface |
| 142 | Nd | CD19 | HIB19 | Fluidigm | 3142001B | Pan B cells | Surface |
| 143 | Nd | CD117 | 104D2 | Fluidigm | 3143001B | Mast cells/p [symbol] | Surface |
| | | (c-kit) | | | | immune cells | |
| 144 | Nd | CD11b | IRCF44 | Fluidigm | 3144001B | Macrophage/monocyte | Surface |
| 145 | Nd | CD4 | RPA-T4 | Fluidigm | 3145001B | T cell subset/monocyte | Surface |
| 146 | Nd | CD8 [symbol] | RPA-T8 | Fluidigm | 3146001B | T cell subset/NK | Surface |
| 147 | Sm | CD11 [symbol] | BU15 | Fluidigm | 3147008B | DC/macrophage/monocyte | Surface |
| 148 | Nd | CD14 | RMO52 | Fluidigm | 3148010B | Macrophage/monocyte | Surface |
| 149 | Sm | CD1 [symbol] | LI61 | BioLegend | Custom | DC | Surface |
| | | (BDCA1) | | | | | |
| 150 | Nd | FcER1 | AER-37 | Fluidigm | 3150027B | DC/pDC/Baso | Surface |
| 151 | E [symbol] | CD123 | 6H6 | Fluidigm | 3151001B | pDC/Base | Surface |
| | | (IL-3Rs) | | | | | |
| 152 | Sm | gdTCR | 11P2 | Fluidigm | 3152008B | gd T cell | Surface |
| 153 | E [symbol] | CD45RA | HI100 | Fluidigm | 3153001B | T cell naïve/memory | Surface |
| 154 | Sm | CD366 | F38-2E2 | Fluidigm | 3154010B | Checkpoint | Surface |
| | | (TIM3) | | | | | |
| 155 | Gd | CD64 | I0.1 | BioLegend | Custom | [symbol] gammaRI | Surface |
| 156 | Gd | CD274 | 29E [symbol] A3 | Fluidigm | 3156026B | Checkpoint | Surface |
| | | (PD-L1) | | | | | |
| 157 | Gd | CD206 | 15-2 | BioLegend | Custom | Macrophage | Surface |
| 158 | Gd | CD17 | L128 | Fluidigm | 3158010B | B/T cell memory | Surface |
| 159 | T [symbol] | CD141 | IA4 | BioLegend | Custom | mDC | Surface |
| 160 | Gd | Tbet | 4B10 | Fluidigm | 3160010B | T [symbol] polarization/NK | Intra- |
| | | | | | | | cellular |
| 161 | Dy | CD152 | 14D3 | Fluidigm | 3161004B | Checkpoint | Intra- |
| | | (CTLA-4) | | | | | cellular |
| 162 | Dy | Foxp3 | PCH101 | Fluidigm | 3162011A | Treg | Intra- |
| | | | | | | | cellular |
| 163 | Dy | CD33 | WM53 | Fluidigm | 3163023B | Pan myeloid | Surface |
| 164 | Dy | CD45RO | UCHL1 | Fluidigm | 3164007B | T cell naïve/memory | Surface |
| 165 | H [symbol] | CD127 | A019D5 | Fluidigm | 3165008B | T cell subset/Treg | Surface |
| | | (IL-7Ra) | | | | | |
| 166 | E [symbol] | CD154 | 24-31 | BioLegend | Custom | T cell activation | Surface |
| | | (CD40L) | | | | | |
| 167 | E [symbol] | CD197 | G043H7 | Fluidigm | 3167009A | T call subset (eff/memory) | Surface |
| | | (CCR7) | | | | | |
| 168 | E [symbol] | K [symbol] 67 | B56 | Fluidigm | 3168007B | Proliferation | Intra- |
| | | | | | | | cellular |
| 169 | Tm | CD25 | 2A3 | Fluidigm | 3169003B | Treg | Surface |
| 170 | E [symbol] | TCR V [symbol] 24- | 6B11 | Fluidigm | 3170015B | iNKT | Surface |
| | | [symbol] 8 | | | | | |
| 171 | Yb | CD40 | 5C3 | BioLegend | Custom | APC | Surface |
| 172 | Yb | CD38 | H [symbol] T2 | Fluidigm | 3172007B | B cell/NK/plasma cell | Surface |
| 173 | Yb | CD192 | K036C2 | BioLegend | Custom | Chemokine Receptor | Surface |
| | | (CCR2) | | | | | |
| 174 | Yb | HLA-DR | L243 | Fluidigm | 3 [symbol] 74001B | APC | Surface |
| 175 | Lu | PD-1 | HP6025 | Selleck Chem/ | Custom | Checkpoint | Surface |
| | | (Ni [symbol] | | South [symbol] Bio | | | |
| | | IgG4 | | | | | |
| 176 | Yb | CD [symbol] 6 | NCAM16.2 | Fluidigm | 3176008B | NK | Surface |
| 2 [symbol] | Bi | CD16 | 3G8 | Fluidigm | 3209002B | [symbol] Receptor/NK/ | Surface |
| | | | | | | Ne [symbol] ophil/M [symbol] | |

[symbol]

indicates data missing or illegible when filed

Data were analyzed using CellEngine™ cloud-based flow cytometry analysis software (CellCarta, Montreal, Quebec, Canada).

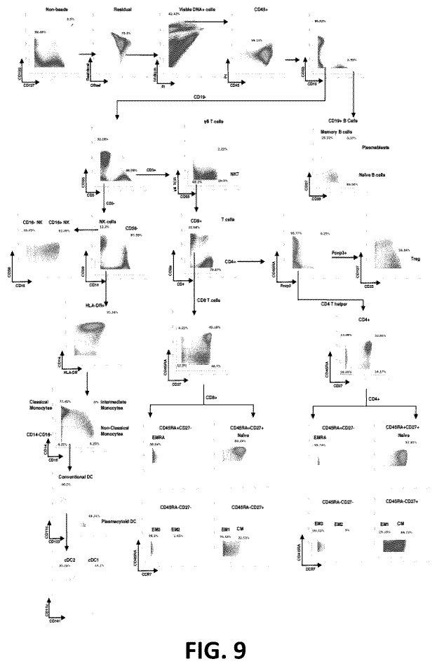

Supervised gating was performed manually by a scientist without reference to clinical outcome. High level gates were tailored per sample. Single marker gates were drawn uniformly for analysis across patients and time points, with example gating strategy provided in FIG. 9. After gating for live singlets, immune populations were defined as following, as shown in FIG. 9. CD4 and CD8 T naïve, effector and memory populations were identified based on CD45RA, CD27 and CCR7 expression. Tregs were identified based on Foxp3, CD25 and CD127 expression. B cells were identified based on CD19 expression and further distinguished into memory vs naïve vs plasmablast based on expression of CD38 vs CD27. NK cells were identified based on CD56 expression and further subdivided based on CD56 vs CD16 expression. Monocytes were identified based on expression of CD14 and HLA-DR and further subdivided in classical, non-classical and intermediate based on the expression of CD14 vs CD16. Dendritic cells were defined as HLA−DR+CD14−CD16− non-lymphocytes and further distinguished between myeloid and plasmacytoid based on expression of CD11c vs CD123, respectively. Myeloid dendritic cells were further subdivided on the basis of CD141 expression into conventional dendritic cells type 1 (cDC1; CD141+) and conventional dendritic cells type 2 (cDC2; CD141−).

In addition to manual gating of defined populations, data was analyzed in an unsupervised fashion. To do this, all samples for all patients and all timepoints were combined together and run through a clustering algorithm^{35,36}. After clustering, clusters were visualized using a force-directed graph layout^{35,36} and colored by association with overall survival. Using this visualization, clusters of interest were identified and then the relevant populations were added to the manual gating hierarchy. All timeseries and survival analyses shown in the results are derived from gated populations, whether discovered by manual gating or unsupervised analysis.

High Parameter Flow Cytometry of T lymphocytes

Cryopreserved PBMC samples for fluorescent flow cytometry were analyzed in the Translational Cytometry Laboratory of the Penn Cytomics and Cell Sorting Shared Resource (University of Pennsylvania, Philadelphia, PA, USA) on an extensively pre-qualified 28-color BD Symphony A5 cytometer (BD Biosciences). Staff were blinded to treatment cohort and clinical outcome. At the time of analysis, cryopreserved PBMC samples were thawed in 37° C. prewarmed RPMI-1640 medium (Gibco) containing 10% FBS and 100 U/ml of penicillin-streptomycin (Gibco). Samples were washed, counted, and resuspended in medium containing 1 mg/mL DNase I (Roche) and 5 mM magnesium chloride, and incubated at 37° C. for 1 h. After resting, cells were washed with PBS without additives (Corning) and transferred to staining tubes. PBMC were incubated with 1 uL (0.2 μg) of 0.2 mg/mL nivolumab antibody (Selleck Chemicals) for 5 min at RT, followed by the addition of a Fixable Viability Stain 510 for 10 min at RT in the dark. Cells were then washed twice with FACS wash buffer (PBS, 1% BSA, 2 mM EDTA). A surface antibody cocktail (T cell phenotyping antibody panel, Table 6 was prepared daily and used to stain up to 1×10⁷ cells per tube.

TABLE 6

| Fluorophore | Target | Clone | Source | Cat # | Category | Staining |
|---|---|---|---|---|---|---|
| BUV395 | CD45RA | HL100 | BD | 740298 | Differentiation | Surface |
| BUV496 | CD8a | RPA-T8 | BD | 612942 | Lineage | Surface |
| BUV583 | CD185 (CXCR5) | RF8B2 | BD | 741316 | Lineage | Surface |
| BUV615 | CD25 | 2A3 | BD | 612996 | Lineage/activation | Surface |
| BUV661 | CD226 (DNAM-1) | DX11 | BD | 749934 | Activation | Surface |
| BUV737 | CD27 | L [symbol] 28 | BD | 612829 | Differentiation | Surface |
| BUV805 | CD4 | SK3 | BD | 612887 | Lineage | Surface |
| BV421 | CD197 (CCR7) | G043H7 | BioLegend | 353208 | Differentiation | Surface |
| BV480 | CD223 (LAG3) | T47-530 | BD | 746609 | Exhaustion | Surface |
| BV510 | Fixable Viability | n/a | BD | 564406 | Dump | Surface |
| | S [symbol] (FV [symbol] ) | | | | | |
| BV510 | CD14 | M5E2 | BD | 740163 | Dump | Surface |
| BV510 | CD19 | SJ25C1 | BD | 562947 | Dump | Surface |
| BV510 | CD4 [symbol] | HIP8 | BD | 563250 | Dump | Surface |
| BV570 | CD3 | UCHT1 | Biolegend | 300436 | Lineage | Surface |
| BV605 | CD137 (4-1BB) | 4B4-1 | BD | 745256 | Activation | Surface |
| BV650 | CD244 (2B4) | 2B4 | BD | 740487 | Activation/ | Surface |
| | | | | | exhaustion | |
| BV711 | CD366 (T [symbol] 3) | 7D3 | BD | 565566 | Exhaustion | Surface |
| BV750 | CD39 | TU66 | BD | 747079 | Exhaustion | Surface |
| BV786 | CD28 | CD28.2 | BD | 740996 | Differentiation | Surface |
| BB515 | CD279 (PD-1) ← | nivolumab, | Selleck Chem; | A2002; | Activation/ | Surface |
| | anti-Human IgG4 | G17-4 | BD | custom | exhaustion | |
| BB660 | CD278 (ICOS) | DX29 | BD | custom | Activation | Surface |
| BB700 | CD127 (IL-7Ra) | HIL-7R-M21 | BD | 566398 | Differentiation | Surface |
| BB790 | CD38 | HIT2 | BD | custom | Differentiation/ | Surface |
| | | | | | activation | |
| PE | TIGIT | MBSA43 | eBioscience | 12-9500- | Exhaustion | Surface |
| | | | | 42 | | |
| PE-eFluor610 | Eomes | WD1928 | eBioscience | 61-4877- | Differentiation/ | Intra-cellular |
| | | | | 42 | exhaustion | |
| PE-Cy5 | CD1 [symbol] 2 (CTLA-4) | BNI3 | BD | 555854 | Exhaustion | Intra-cellular |
| PE-Cy5.5 | FoxP3 | PCH101 | eBioscience | 35-4776- | Lineage | Intra-cellular |
| | | | | 42 | | |
| PE-Cy7 | T-bet | O4-46 | BD | custom | Lineage/activation | Intra-cellular |
| AF647 | TCF-1 (TCF7) | S33-966 | BD | 566693 | Differentiation | Intra-cellular |
| AF700 | Ki-67 | B56 | BD | 561277 | Proliferation | Intra-cellular |
| APC-Fire750 | KLRG1 | SA231A2 | Biolegend | 367718 | Exhaustion | Surface |

[symbol]

indicates data missing or illegible when filed

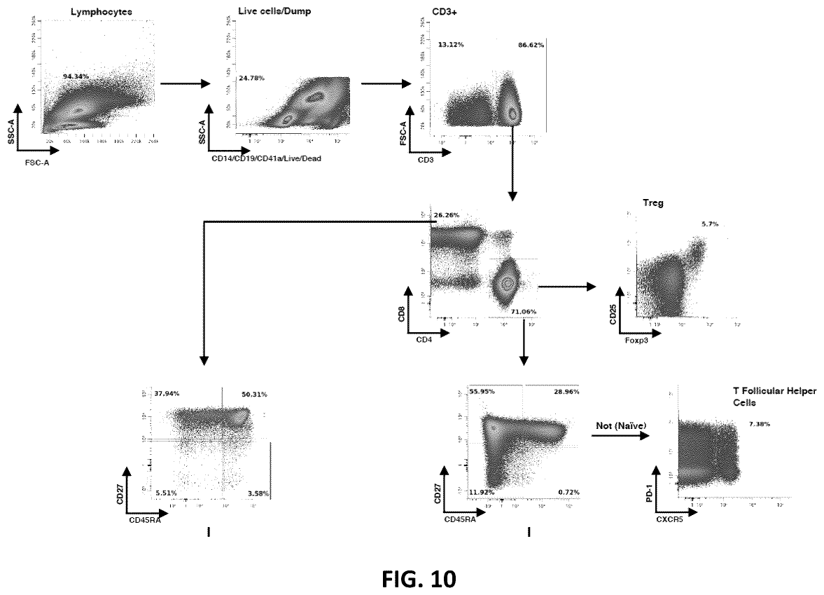

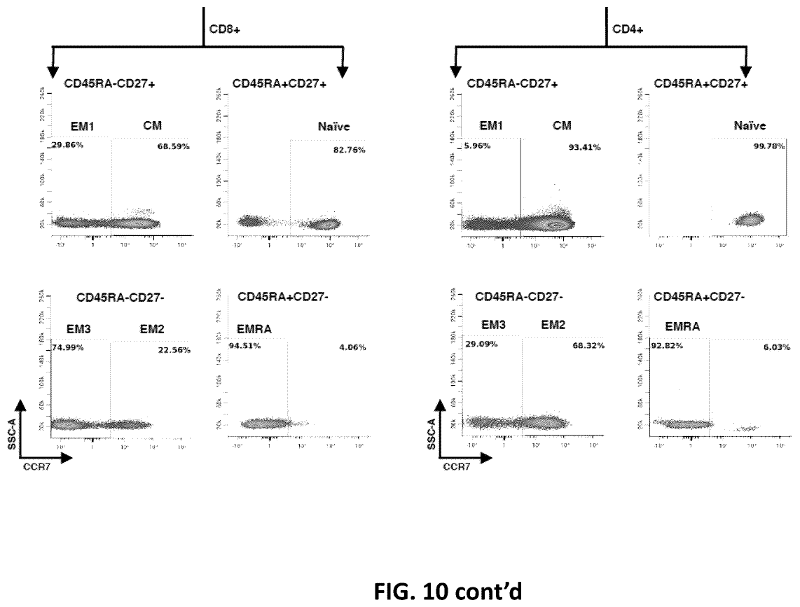

Cells were incubated for 20 minutes at RT followed by washing twice with FACS staining buffer. The cells were resuspended in FoxP3 Transcription Factor Staining Buffer Fix/Perm solution (eBiosciences) and incubated for 1 hour at RT to prepare the cells for intracellular staining. Post fixation, the samples were washed with Foxp3 permeabilization buffer. A freshly prepared cytoplasmic/intracellular staining cocktail master mix was added to the samples and incubated overnight at 4° C. The following day, the samples were washed with permeabilization buffer and resuspended in FACS wash buffer. Cells were stored at 4° C. in the dark and acquired within 2 hours. Following daily QC the instrument was standardized by setting hard dyed beads (BD Biosciences, Cytometer Setup and Tracking Beads (CS&T)) to predetermined target channels. Compensation controls (Invitrogen UltraComp eBeads or cells for Live/Dead stain) were prepared daily along with a frozen PBMC process control. The compensation matrix was calculated in Diva software (BD Biosciences) and used only for that day's run. Data were analyzed using CellEngine™ cloud-based flow cytometry analysis software (CellCarta, Montreal, Quebec, Canada). High level gates were tailored per patient across all time points by at least two investigators blinded to patient outcome. Single marker gates were drawn uniformly for analysis across patients and time points, with representative gating strategy provided in FIG. 10.

After gating for live cells and the CD3+ population, T cell populations were defined as following, as shown in FIG. 10, a combination of CD45RA, CD27 and CCR7 expression on CD4+ and CD8+ T cells was used to define naïve (CD45RA+CD27+CCR7+), T central memory (CM; CD45RA−CD27+CCR7+), T effector memory 1 (EM1; CD45RA−CD27+CCR7−), T effector memory 2 (EM2; CD45RA−CD27−CCR7+), T effector memory 3 (EM3; CD45RA−CD27−CCR7−), and Terminally Differentiated Effector Memory (EMRA) (CD45RA+CD27⁻CCR7−) subpopulations. CD4+ regulatory T cells were defined as Foxp3+CD25^{hi}CD127^{-/low}. The non-naïve CD4+ and CD8⁺ T cell populations used in timeseries and survival analyses included the defined effector memory, central memory, and TEMRA populations defined above. Expression of additional differentiation, activation and inhibitory markers were evaluated within each of these compartments.

In addition to manual gating of defined populations, data was analyzed in an unsupervised fashion. To do this, all samples for all patients and all timepoints were combined together and run through a clustering algorithm as described in^{35,36}. After clustering, clusters were visualized using a force-directed graph layout^{35,36} and colored by association with overall survival. Using this visualization, clusters of interest were identified and then the relevant populations were added to the manual gating hierarchy. All timeseries and survival analyses shown in the results are derived from gated populations, whether discovered by manual gating or unsupervised analysis.

### Serum Proteomics Profiling

Serum proteins were quantified using Olink multiplex proximity extension assay (PEA) panels (Olink Proteomics; www.olink.com) according to the manufacturer's instructions and as described before³⁷. The assay was performed at the Olink Analysis Service Center (Boston, MA, USA). The basis of PEA is a dual-recognition immunoassay, where two matched antibodies labelled with unique DNA oligonucleotides simultaneously bind to a target protein in solution. This brings the two antibodies into proximity, allowing their DNA oligonucleotides to hybridize, serving as template for a DNA polymerase-dependent extension step. This creates a double-stranded DNA “barcode” which is unique for the specific antigen and quantitatively proportional to the initial concentration of target protein. The hybridization and extension are immediately followed by PCR amplification and the amplicon is then finally quantified by microfluidic qPCR using Fluidigm BioMark HD system (Fluidigm Corporation, South San Francisco, CA). Data were normalized using internal controls in every single sample, inter-plate control and negative controls and correction factor, and expressed as Log2-scale which is proportional to the protein concentration. The final assay readout is reported as normalized protein expression (NPX) values, which is an arbitrary unit on a log 2-scale where a higher value corresponds to a higher protein expression. One NPX difference equals to the doubling of the protein concentration. In this study, two Olink panels (Target96 Immuno-Oncology and Target96 Immune Response) were used which consist of 172 unique analytes. Additional details about the analytes, detection range, data normalization and standardization are available at https://www.olink.com/resources-support/document-download-center/.

### Unbiased Mass Spectrometry Serum Profiling

Serum samples were profiled using a high-throughput quantitative proteomics workflow for over 1600 quantifiable proteins at Biognosys (Schlieren-Zurich, Switzerland). All samples were handled and thawed equally. During the aliquoting, a small amount of each sample was pooled and used as a quality control sample for subsequent library generation and to assess quality and batch effects throughout the sample preparation and acquisition. Three processing batches were block randomized for treatment and site (samples coming from one patient were kept within the same batch but randomized across it). The automated depletion pipeline composed of sequential depletion, parallel digestion and liquid chromatography (LC)-mass spectrometry (MS) acquisition was performed as previously reported³⁸. Quality control samples were depleted within each processing batch. Both data-independent acquisition (DIA) LC-MS and data-dependent acquisition (DDA) LC-MS/MS measurements were acquired. DIA and DDA mass spectrometric data were analyzed using the software SpectroMine (version 3.0.2101115.47784, Biognosys) using the default settings, including a 1% false discovery rate control at PSM, peptide and protein level, allowing for 2 missed cleavages and variable modifications (N-term acetylation and methionine oxidation). The human UniProt.fasta database (Homo sapiens, 2020-01-01, 20,367 entries) was used and for the library generation, the default settings were used.

Raw mass spectrometric data were analyzed using the software Spectronaut (version 14.7.201007.47784, Biognosys) with the default settings, but Qvalue sparse filtering was enabled with a global imputing strategy and a hybrid library comprising all DIA and DDA runs conducted in this study³⁹. Default settings include peptide and protein level false discovery rate control at 1% and cross-run normalization using global normalization on the median. Protein wise mean normalization based on the 80% quantile of the QC samples between batches 1 and 2-3 removed the identified batch effect by both principal component analysis (PCA, ‘stats’ R-package) or hierarchical clustering.

### Whole Exome and Transcriptome Sequencing

FFPE tumor and normal PBMC samples were profiled using ImmunoID NeXT (Personalis, Inc., Menlo Park, CA, USA); an augmented exome/transcriptome platform and analysis pipeline, which produces comprehensive tumor mutation information, gene expression quantification, neoantigen characterization, HLA typing and allele specific HLA loss of heterozygosity data (HLA LOH), TCR repertoire profiling and tumor microenvironment profiling. Whole exome library preparation and sequencing was performed as previously described⁴⁰. DNA extracted from tumor and PBMCs was used to generate whole-exome capture libraries using the KAPA HyperPrep Kit and Agilent's SureSelect Target Enrichment Kit, according to manufacturers' recommendations, with the following amendments: 1) Target probes were used to enhance coverage of biomedically and clinically relevant genes. 2) Protocols were modified to yield an average library insert length of approximately 250 bp. 3) KAPA HiFi DNA Polymerase (Kapa Biosystems) was used in place of Herculase II DNA polymerase (Agilent). Paired-end sequencing was performed on NovaSeq instrumentation (Illumina, San Diego, CA, USA). Paired-end sequencing was performed on NovaSeq instrumentation (Illumina, San Diego, CA, USA).

Whole-transcriptome sequencing results were aligned using STAR⁴¹ and normalized expression values in transcripts per million (TPM) calculated using Personalis' ImmunoID NeXT tool, Expressionist. For RNA sequencing and alignment quality control, the following metrics were evaluated: average read length, average mapped read pair length, percentage of uniquely mapped reads, number of splice sites, mismatch rate per base, deletion/insertion rate per base, mean deletion/insertion length, and anomalous read pair alignments including inter-chromosomal and orphaned reads. The ImmunoID NEXT DNA and RNA Analysis Pipeline aligns reads to the hs37d5 reference genome build. The pipeline performs alignment, duplicate removal, and base quality score recalibration using best practices outlined by the Broad Institute^{42,43}. The pipeline uses Picard to remove duplicates and Genome Analysis Toolkit (GATK) to improve sequence alignment, and correct base quality scores (BQSR). Aligned sequence data is returned in BAM format according to SAM specification. Raw read counts from were also normalized using R to get weighted trimmed mean of the log expression ratios (trimmed mean of M values (TMM)).

To calculate gene expression signatures on a given geneset, scores were determined via geometric mean of the normalized count values of respective gene signatures. Patient tumor samples were collected from a range of primary tumors and metastatic sites. Due to the impact on tissue of origin on bulk RNAseq, we chose to limit all gene expression analyses to the most common biopsy site, liver metastases, which constituted roughly 64% of biopsies. All gene expression analyses thus include only biopsies from liver metastases, see Table 7 for counts.

TABLE 7

| Assay | Treatment Arm: Timepoint (# of samples) |
|---|---|
| X50 | nivo/chemo: C1D1 (n = 26), C1D15 (n = 21), C2D1 (n = 25), C4D1 (n = 19) |
| | sotiga/chemo: C1D1 (n = 28), C1D15 (n = 23), C2D1 (n = 27), C4D1 (n = 18) |
| | sotiga/nivo/chemo: C1D1 (n = 32), C1D15 (n = 27), C2D1 (n = 29), C4D1 (n = 14) |
| CyTOF | nivo/chemio: C1D1 (n = 25), C1D15 (n = 20), C2D1 (n = 23), C4D1 (n = 13) |
| | sotiga/chemo: C1D1 (n = 29), C1D15 (n = 23), C2D1 (n = 24), C4D1 (n = 22) |
| | sotiga/nivo/chemo: C1D1 (n = 26), C1D15 (n = 20), C2D1 (n = 26), C4D1 (n = 13) |
| Olink Platform | nivo/chemo: C1D1 (n = 32), C1D15 (n = 25), C2D1 (n = 27), C3D1 (n = 25), C4D1 (n = 23) |
| | sotiga/chemo: C1D1 (n = 36), C1D15 (n = 29), C2D1 (n = 31), C3D1 (n = 25), C4D1 (n = 27) |
| | sotiga/nivo/chemo: C1D1 (n = 35), C1D15 (n = 27), C2D1 (n = 32), C3D1 (n = 26), C4D1 (n = 25) |
| Biognosys | nivo/chemo: C1D1 (n = 30), C1D15 (n = 25), C2D1 (n = 19), C3D1 (n = 18) |
| | sotiga/chemo: C1D1 (n = 32), C1D15 (n = 27), C2D1 (n = 28), C3D1 (n = 22) |
| RNAseq | nivo/chemo: pretreatment (n = 25), liver biopsies (n = 17) |
| | sotiga/chemo: pretreatment (n = 18), liver biopsies (n = 12) |
| | sotiga/nivo/chemo: pretreatment (n = 23), liver biopsies (n = 12) |

### Multiplex Tissue Staining and Imaging

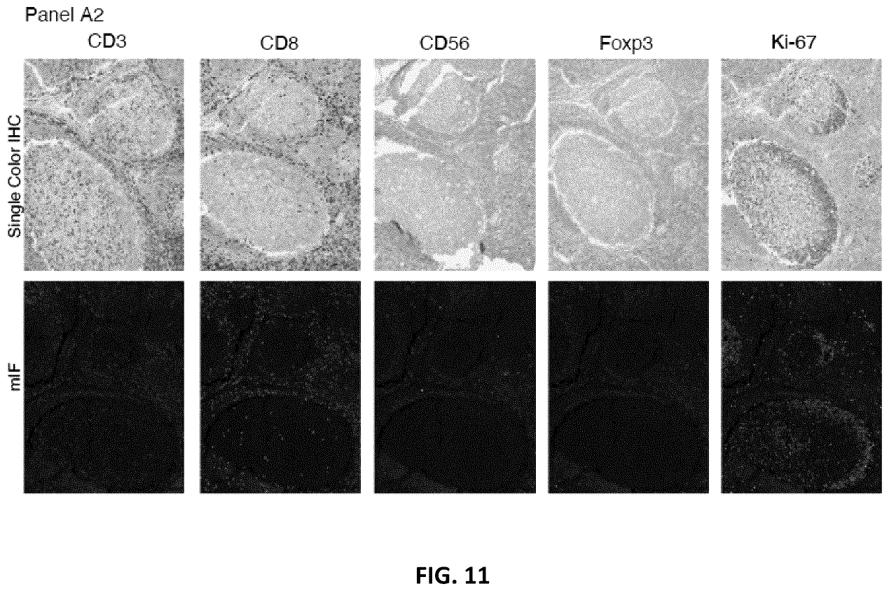

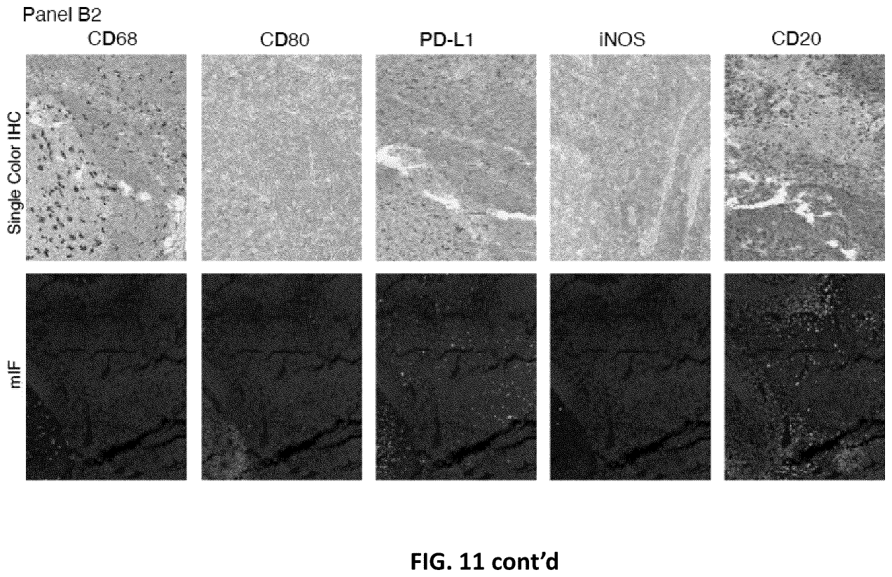

Tumor tissue was collected prior to treatment (fresh baseline biopsy or archival tissue), on-treatment (during cycle 2), and optionally at the end of treatment. Tissues were fixed in formalin followed by paraffin-embedding. All tissue imaging was performed under the guidance of an expert pathologist (TJH) in the Advanced Immunomorphology Platform Laboratory at Memorial Sloan Kettering Cancer Center (New York, NY). Primary antibody staining conditions were optimized using standard immunohistochemical staining on the Leica Bond RX automated research stainer with DAB detection (Leica Bond Polymer Refine Detection DS9800). Using 4 μm tissue sections and serial antibody titrations on control tonsil tissue, the optimal antibody concentration was determined followed by transition to a seven-color multiplex assay with equivalency (see FIG. 11 for control staining). Four antibody panels were utilized for staining. Panels A1 and B1 were used for tissues collected in Phase 1b. Panels A2 and B2 were further optimized for distribution of cellular markers and were used for tissues collected in Phase 2. Multiplex assay antibodies and conditions are described in Table 8.

TABLE 8

| | | | | |
|---|---|---|---|---|
| | Antibody | | | Detection |
| Marker | clone | Source | Dilution | Dye (cycle) |

Panel A1

| CD8 | C8/114B | CST | 0.042 | ug/mL | Opal 520 (1) |
|---|---|---|---|---|---|
| Ki67 | SP6 | Biocare | 1:100 | | Opal 540 (2) |
| CD68 | PG-M1 | Dako | 0.15 | ug/mL | Opal 570 (3) |
| FOXP3 | 236A/E7 | Biocare | 1:2 | | Opal 620 (4) |
| PDL1 | 73-10 | Abcam | 0.18 | ug/mL | Opal 650 (5) |
| PanCK | AE1/AE3 | DAKO | 0.665 | ug/mL | Opal 690 (6) |

Panel B1

| CD3 | BC33 | Biocare | 1:200 | | Opal 520 (1) |
|---|---|---|---|---|---|
| INOS | 13F5.1 | Millipore | 1:2500 | | Opal 540 (2) |
| Granzyme B | EPR8260 | Abcam | 1:100 | | Opal 570 (3) |
| CD20 | E7B7T | CST | 0.055 | ug/mL | Opal 620 (4) |
| CD56 | MRQ-42 | CellMarque | 1:2 | | Opal 650 (5) |
| panCK | AE1/AE3 | DAKO | 0.665 | ug/mL | Opal 690 (6) |

Panel A2

| CD3 | BC33 | Biocare | 1:200 | | Opal 520 (1) |
|---|---|---|---|---|---|
| Ki67 | SP6 | Biocare | 1:100 | | Opal 540 (2) |
| CD56 | MRQ-42 | CellMarque | 1:2 | | Opal 570 (3) |
| FOXP3 | 236A/E7 | Biocare | 1:2 | | Opal 620 (4) |
| CD8 | C8/114B | CST | 0.042 | ug/mL | Opal 650 (5) |
| panCK | AE1/AE3 | DAKO | 0.665 | ug/mL | Opal 690 (6) |

Panel B2

| CD80 | EPR1157(2) | Abcam | 1:500 | | Opal 520 (1) |
|---|---|---|---|---|---|
| INOS | 13F5.1 | Millipore | 1:2500 | | Opal 540 (2) |
| CD68 | PG-M1 | Dako | 0.15 | ug/mL | Opal 570 (3) |
| CD20 | E7B7T | CST | 0.055 | ug/mL | Opal 620 (4) |
| PDL1 | 73-10 | Abcam | 0.18 | ug/mL | Opal 650 (5) |
| panCK | AE1/E3 | DAKO | 0.665 | ug/mL | Opal 690 (6) |

Seven color multiplex imaging assay. FFPE tissue sections were baked for 3 h at 62° C. in a vertical slide orientation with subsequent deparaffinization performed on the Leica Bond RX followed by 30-min of antigen retrieval with Leica Bond ER2 followed by 6 sequential cycles of staining with each round including a 30-min combined block and primary antibody incubation (Akoya antibody diluent/block). For Ki67 and panCK, detection was performed using a secondary horseradish peroxidase (HRP)-conjugated polymer (Akoya Opal polymer HRP Ms+Rb; 10-minute incubation). Detection of all other primary antibodies was performed using a goat anti-mouse Poly HRP secondary antibody or goat anti-rabbit Poly HRP secondary antibody (Invitrogen; 10-min incubation). The HRP-conjugated secondary antibody polymer was detected using fluorescent tyramide signal amplification using Opal dyes 520, 540, 570, 620, 650 and 690 (Akoya Biosciences, Marlborough, MA). The covalent tyramide reaction was followed by heat induced stripping of the primary/secondary antibody complex using Akoya AR9 buffer and Leica Bond ER2 (90% AR9 and 10% ER2) at 100° C. for 20 min preceding the next cycle. After 6 sequential rounds of staining, sections were stained with Hoechst 33342 (Invitrogen) to visualize nuclei and mounted with ProLong Gold antifade reagent mounting medium (Invitrogen).

Multispectral imaging and spectral unmixing. Seven color multiplex stained slides were imaged using the Vectra Multispectral Imaging System version 3 (Akoya). Scanning was performed at 20× (200× final magnification). Filter cubes used for multispectral imaging were DAPI, FITC, Cy3, Texas Red and Cy5. A spectral library containing the emitted spectral peaks of the fluorophores in this study was created using the Vectra image analysis software (Akoya). Using multispectral images from single-stained slides for each marker, the spectral library was used to separate each multispectral cube into individual components (spectral unmixing) allowing for identification of the seven marker channels of interest using Inform 2.4 image analysis software.

mIF image analysis. Individual region of interest (ROI) images were exported to TIFF files and run through a pipeline for multiplexed imaging quality control and processing under the supervision of an expert pathologist. A machine-learning cell segmentation algorithm was used to segment individual whole cells along the membrane border using nuclear as well as multiple membrane markers to enable drawing borders for all cell types. For each cell segment, pixel values within each region were averaged to give a single intensity value per cell and per marker. Using these single-cell intensity values, cell type assignments were made manually by a scientist determining cutoff points for positive marker expression for each sample. To do this manual thresholding, the distribution of single-cell marker values and the appearance of fluorescence on the images themselves were simultaneously inspected using the CellEngine™ software (CellCarta) alongside Mantis Viewer, a custom in-house open-source software used for fluorescent image visualization (http://doi.org/10.5281/zenodo.4009579) and thresholds for each marker were drawn per sample. Using these individual marker thresholds, cell types were defined by positivity of combined associated markers in the panel as described in Table 9.

TABLE 9

| Cell Population | Marker Expression |
|---|---|
| B Cells | CD68−, CD20+ |
| Macrophages | CD68+ |
| iNOS+ Macrophages | CD68+, iNOS+ |
| CD80+ Macrophages | CD68+, CD80+ |
| iNOS+ CD80+ Macrophages | CD68+, iNOS+, CD80+ |
| PD-L1+ Macrophages | CD68+, PD-L1+ |
| NK Cells | CD3−, CD56+ |
| T Cells | CD3+ |
| CD8 T Cells | CD3+, CD8+ |
| CD4 T Cells | CD3+, CD8− |
| Ki-67- CD4 T cells | CD3+, CD8−, FoxP3−, Ki-67− |
| T Regulatory Cells | CD3+, CD8−, FoxP3+ |
| Ki-67+ T Regulatory Cells | CD3+, CD8−, FoxP3+, Ki-67+ |
| Tumor Cells | PanCK+ |
| PD-L1+ Tumor Cells | PanCK+, PD-L1+ |

Once cell types were defined, the percentage out of total cells and out of the parent population was calculated for each ROI. Then, for each sample, the median across ROIs was taken for percent of total cells, percent of parent population, and occasionally percent of other relevant populations.

Analysis of all Data for Association with Survival and Pharmacodynamic Changes

Data storage and structure All processed biomarker data was combined with cleaned clinical data and loaded into a proprietary in-house database named the Cancer Data & Evidence Library (CANDEL)⁴⁴. CANDEL uses the database technology Datomic™ (www.datomic.com) and a suite of tools built to enable storage of molecular and clinical data and fast query and visualization from the R programming language.

Data analysis in R All molecular data was analyzed for association with outcomes and treatment using the R programming language (R Foundation for Statistical Computing)⁴⁵ with the packages and versions listed in Table 10.

TABLE 10

| Package Name | Version |
|---|---|
| Ggplot2 | 3.3.5 |
| wick | 1.1 |
| suvminer | 0.4.9 |
| dplyr | 1.0.7 |
| plyr | 1.8.6 |
| Reshape2 | 1.4.4 |
| Data.table | 1.14.0 |
| survival | 3.2-13 |
| tidyr | 1.1.4 |
| tidyverse | 1.3.1 |
| ggpubr | 0.4.0 |
| limma | 3.48.3 |
| readxl | 1.3.1 |
| msigdbr | 7.4.1 |
| stringr | 1.4.0 |
| venn | 1.10 |
| mixOmics | 6.16.3 |
| pheatmap | 1.0.12 |
| readr | 2.0.1 |

Association with survival was analyzed for cell population percentages, protein values, and gene expression signatures by separating patients into two groups based on the median value across all patients in all cohorts. Between these two groups, for each cohort, Kaplan-Meier plots were created and log-rank p-value significance was determined using the survminer and survival packages. To visualize differences between any defined groups or visualize changes on treatment, Ggplot2 and base R plotting were used. To determine differences between pretreatment and on-treatment values as well as differences between survival groups (>1 year and <1 year) at any given timepoint, a Wilcoxon sign-rank test with a significance cutoff of p=0.05 was used. Median log fold change was calculated to determine additional pharmacodynamic differences seen from pretreatment to on-treatment. Heatmaps and circus plots for multi-omic analysis were generated using the DIABLO method in the mixOmics R package. Heatmaps were generated using pheatmap and correlations across data types were calculated using the Spearman method.

Associative analysis of mass spectrometry data Due to the large number of proteins analyzed, additional methods were used for mass spectrometry data. Initial univariate candidate filtering was performed using pairwise Wilcoxon test applied per protein across cohorts with Holmes-Bonferroni correction (within-group). Proteins with a p-value below or equal 0.05 from randomly selected 80% of observations were used for further optimization using the sparse partial least square discriminant analysis (sPLS-DA) approach with zero as a threshold for absolute feature importance⁴⁶. The ratio between C1D1 and C1D15, C2D1, and C3D1 was calculated and further used downstream. Randomly selected 80% of observations were used for sPLS-DA for all cohort arms. A leave-one-out algorithm was used for optimal component and protein selection. sPLSDA training and testing were performed using the R-package ‘mixOmics’⁴⁷. The remaining 20% of observations were used for validation. Accuracy of prediction for all three groups, C1D15, C2D1, and C3D1, were calculated as the ratio of the true positive and negative-sum to all observations (R-package ‘caret’). Unsupervised hierarchical analysis was done with Manhattan distance and Ward's clustering on centered and normalized data (xij-x̅ j/sj, i-th observation with j-th protein) using R-package ‘ComplexHeatmap’. PCA analysis was done using R-package ‘stats’. Correlation analysis was done using Pearson correlation with R-packages ‘stats’ and ‘corrplot’. Correlation significance was tested using a two-sided t-test at 0.05 alpha. All analyses were performed using log 2 transformed data.

### Example 7. Baseline Demographics and Disease Characteristics

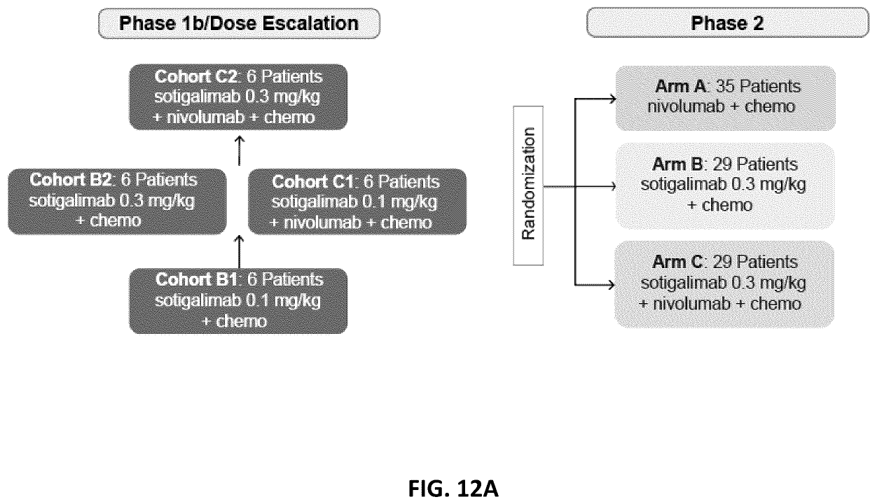

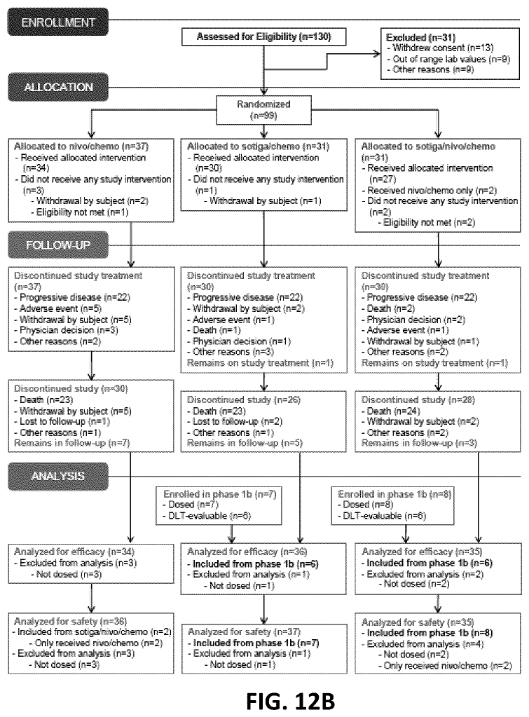

From Aug. 30, 2018, through Jun. 10, 2019, 99 patients were randomly allocated into one of three treatment arms (N=37, 31, and 31 to nivo/chemo, sotiga/chemo, sotiga/nivo/chemo, respectively; FIG. 12 (Six patients (N=3, 1, and 2, respectively) were randomized but not dosed and were excluded from analysis FIG. 12. Efficacy was assessed for 105 patients (N=34, 36, 35), which included 93 patients randomized and dosed in Phase II and 12 DLT-evaluable patients from the Phase Ib study¹³ (6 each on sotiga/chemo and sotiga/nivo/chemo). Safety was assessed for 108 patients (N=36, 37, 35, respectively), which included the 105 patients assessed for efficacy plus 3 non-DLT-evaluable patients from phase 1b. The clinical snapshot data for analysis was Mar. 24, 2021.

Baseline characteristics for the efficacy population were generally balanced across arms, including age, sex, race/ethnicity, primary pancreatic tumor location, site of metastatic spread, stage of diagnosis, and tumor burden (Table 11, Table 12).

TABLE 11
Extended Table 1. Demographic and Baseline Disease Characteristics
for Patients in the Efficacy Population.

| | | |
|---|---|---|
| Nivolumab + | Sotigalimab + | Sotigalimab + |
| Chemo | Chemo | Nivolumab + Chemo |
| (N = 34) | (N = 36) | (N = 35) |

Characteristic
Age - years

| Median (range) | 62.5 | (47-75) | 60.5 | (35-78) | 62.0 | (41-78) |
|---|---|---|---|---|---|---|
| ≥65 years, n (%) | 14 | (41) | 14 | (39) | 13 | (37) |
| Sex, n (%) | | | | | | |
| Female | 14 | (41) | 13 | (36) | 16 | (46) |
| Male | 20 | (59) | 23 | (64) | 19 | (54) |
| Race and ethnic group, n (%) | | | | | | |
| Asian | 3 | (9) | 4 | (11) | 0 | |
| Black | 0 | | 3 | (8) | 2 | (6) |
| White | 29 | (85) | 28 | (78) | 31 | (69) |
| Other | 2 | (6) | 1 | (3) | 2 | (6) |
| Hispanic | 1 | (3) | 1 | (3) | 1 | (3) |
| ECOG performance status, n (%) | | | | | | |
| 0 | 15 | (44) | 20 | (56) | 15 | (43) |
| 1 | 19 | (56) | 16 | (44) | 20 | (57) |
| Pancreatic tumor location, n (%) | | | | | | |
| Head | 14 | (41) | 17 | (47) | 19 | (54) |
| Body | 12 | (35) | 9 | (25) | 10 | (29) |
| Tail | 8 | (24) | 10 | (28) | 6 | (17) |
| Select sites of metastatic disease, n (%) | | | | | | |
| Liver | 28 | (82) | 29 | (81) | 27 | (77) |
| Lung | 10 | (29) | 10 | (28) | 11 | (31) |
| Peritoneum | 8 | (24) | 9 | (25) | 11 | (31) |
| Stage at initial PDAC diagnoses, n (%) | | | | | | |
| Stages I-III | 7 | (21) | 9 | (25) | 9 | (26) |
| Stage IV | 27 | (79) | 27 | (75) | 26 | (74) |
| Time from diagnosis to first dose - | 1.1 | (0.4-69.8) | 1.0 | (0.4-39.1) | 1.1 | (0.4-45.3) |
| months, median (range)^{a} | | | | | | |
| Prior cancer treatment, n (%) | | | | | | |
| Chemotherapy | 9 | (27) | 7 | (19) | 6 | (17) |
| Radiation therapy | 7 | (21) | 1 | (3) | 4 | (11) |
| Surgery | 11 | (32) | 11 | (31) | 8 | (23) |

| | | | |
|---|---|---|---|
| Tumor burden, mm^{b} | | | |
| Median | 78.5 | 68.5 | 79.0 |
| Range | 18-180 | 19-214 | 10-194 |

Abbreviations:
chemo = chemotherapy; ECOG = Eastern Cooperative Oncology Group; mm = millimeter; PDAC = pancreatic ductal adenocarcinoma.
* Includes all randomized and dosed patients in phase 2 and DLT-evaluable patients from phase 1b enrolled at the recommended phase 2 dose of sotigalimab.
^{a}Calculations exclude one participant from nivo/chemo who did not report a date of diagnosis.
^{b}Tumor burden is the sum of the largest diameters of all target lesions (shortest diameter for lymph nodes).

TABLE 12

| | | |
|---|---|---|
| Nivolumab + | Sotigalimab + | Sotigalimab + |
| Chemo | Chemo | Nivolumab + Chemo |
| (N = 36) | (N = 37) | (N = 35) |

Characteristic
Age - years

| Median (range) | 61.5 | (41-75) | 61.0 | (35-78) | 62.0 | (39-78) |
|---|---|---|---|---|---|---|
| ≥65 years, n (%) | 14 | (39) | 15 | (41) | 14 | (40) |
| Sex, n (%) | | | | | | |
| Female | 14 | (39) | 13 | (35) | 17 | (49) |
| Male | 22 | (61) | 24 | (65) | 18 | (51) |
| Race and ethnic group, n (%) | | | | | | |
| Asian | 3 | (8) | 4 | (11) | 1 | (3) |
| Black | 0 | | 3 | (8) | 2 | (6) |
| White | 31 | (86) | 29 | (78) | 30 | (86) |
| Other | 2 | (6) | 1 | (3) | 2 | (6) |
| Hispanic | 1 | (2) | 1 | (3) | 1 | (3) |
| ECOG performance status, n (%) | | | | | | |
| 0 | 16 | (44) | 20 | (54) | 16 | (46) |
| 1 | 20 | (56) | 17 | (46) | 19 | (54) |
| Pancreatic tumor location, n (%) | | | | | | |
| Head | 15 | (42) | 17 | (46) | 19 | (54) |
| Body | 13 | (36) | 10 | (27) | 9 | (26) |
| Tail | 8 | (22) | 10 | (27) | 7 | (20) |
| Select sites of metastatic disease, n (%) | | | | | | |
| Liver | 29 | (81) | 30 | (81) | 27 | (77) |
| Lung | 11 | (31) | 11 | (30) | 11 | (31) |
| Peritoneum | 9 | (25) | 10 | (27) | 11 | (31) |
| Stage at initial PDAC diagnosis, n (%) | | | | | | |
| Stages I-III | 8 | (22) | 9 | (24) | 9 | (26) |
| Stage IV | 28 | (78) | 28 | (76) | 26 | (74) |
| Time from diagnosis to first dose - | 1.3 | (0.4-69.8) | 1.0 | (0.2-29.1) | 1.1 | (0.4-29.6) |
| months, median (range)° | | | | | | |
| Prior cancer treatment, n (%) | | | | | | |
| Chemotherapy | 10 | (28) | 7 | (19) | 6 | (17) |
| Radiation therapy | 7 | (19) | 1 | (3) | 5 | (14) |
| Surgery | 12 | (33) | 11 | (30) | 8 | (23) |

Abbreviations:
chemo = chemotherapy; ECOG = Eastern Cooperative Oncology Group; PDAC = pancreatic ductal adenocarcinoma.
* Includes all phase 1b and phase 2 patients who received at least 1 dose of any study drug. For safety analyses, patients were grouped according to the study treatment actually received.
°Calculations exclude one participant from nivo/chemo who did not report a date of diagnosis.

A higher proportion of patients on sotiga/chemo had an ECOG score of 0 at screening (44%. 56%, and 43% in nivo/chemo, sotiga/chemo, and sotiga/nivo/chemo, respectively). Across arms. 74-79% of patients had de novo Stage IV disease.

Pretreatment PD-L1+ tumor percentages by multiplex immunofluorescent imaging (mIF) were similar between the nivo/chemo and sotiga/nivo/chemo arms but less in the sotiga/chemo arm Table 13.

TABLE 13

| | Nivolumab + | | Sotigalimab + | | Sotigalimab + | |
|---|---|---|---|---|---|---|
| | Chemo | | Chemo | | Nivolumab + Chemo | |
| Characteristic | (N = 34) | | (N = 36) | | (N = 35) | |
| PD−L1+ tumor percentage^{a} | | | | | | |
| >1%^{b}, n/number evaluable (%) | 10/18 | (56) | 7/19 | (37) | 14/24 | (58) |
| Tumor mutation data available^{b}, n (%) | 27 | (79) | 25 | (69) | 22 | (63) |
| KRAS, n/number evaluable^{c} (%) | 19/27 | (70) | 14/25 | (64) | 13/22 | (59) |
| G12D mutation | 9/27 | (33) | 6/25 | (24) | 3/22 | (14) |
| G12R mutation | 3/27 | (11) | 1/25 | (4) | 2/22 | (9) |
| G12V mutation | 6/27 | (22) | 6/25 | (24) | 6/22 | (27) |
| Q61H mutation | 1/27 | (4) | 2/25 | (8) | 1/22 | (5) |
| Q61R mutation | 0/27 | (0) | 1/25 | (4) | 1/22 | (5) |
| MSI-high, n/number evaluable (%) | 1/27 | (1) | 0/25 | (0) | 1/22 | (5) |
| BRCA1, n/number evaluable (%) | 3/27 | (11) | 0/25 | (0) | 3/22 | (14) |
| BRCA2, n/number evaluable (%) | 0/27 | (0) | 1/25 | (4) | 0/22 | (0) |
| EGFR, n/number evaluable (%) | 3/27 | (11) | 0/25 | (0) | 0/22 | (0) |
| SMAD, n/number evaluable (%) | 4/27 | (15) | 3/25 | (12) | 222 | (9) |
| TP53, n/number evaluable (%) | 19/27 | (70) | 7/25 | (28) | 14/22 | (64) |

Abbreviations: chemo = chemotherapy; MSI = microsatellite instability.
*Includes all randomized and dosed patients in phase 2 and DLT-evaluable patients from phase 1b enrolled at the recommended phase 2 dose of sotigalimab.
^{a}PD−L1+ was assayed with a multiplex research assay and timor percentage was calculated in a method most similar to the Combined Positive Score (CPS). However, PD−L1+ tumor percentages were assessed by multiplex IHC on multiple regions of interest on a single FFPE tumor sample slide analyzed by computational methods and, thus, are not directly comparable to single-marker IHC assays assessed by a trained pathologist.
^{b}Data are unavailable for honor mutation analysis due to not having a pre-treatment tumor sample of sufficient quality for DNA sequencing for 11, 17, and 14 patients in the nivo/chemo, sotiga/chemo, and sotiga/nivo/chemo arms, respectively.
^{c}No other KRAS mutations detected.

Seventy-four (70%) patients had pretreatment tumor tissue of high enough quality for Whole Exome Sequencing (WES) available. By WES, treatment arms were balanced for oncogene frequencies in KRAS, BRCA1/2, SMAD4, and TP53 in mPDAC (Supplementary Table 2). Most patients (62%) had KRAS-mutant tumors. The tumor tissue for 1 patient (in nivo/chemo) was microsatellite instability-high. Seven patients (3 in nivo/chemo, 1 in sotiga/chemo, 3 in sotiga/nivo/chemo) had BRCA mutations detected in the tumor. Additionally, the arms were relatively balanced for gene expression signatures in pre-treatment tumor tissues and had similar baseline frequencies of immune cell populations within circulation.

### Example 8. Follow-Up Drug Exposure

At the time of analysis, the median duration of follow-up for patients in the efficacy population was 24.2 months (interquartile range [IQR] 20.5-26.3) with minimum follow-up of 15 months. Two patients remain on treatment, one each on sotiga/chemo and sotiga/nivo/chemo. Median time on treatment was similar between the 3 arms (median (IQR), months: 5.2 (1.9-8.1), 5.1 (3.4-8.9), 4.7 (2.4-7.9) months for nivo/chemo, sotiga/chemo, sotiga/nivo/chemo, respectively). Exposure to each drug in the combination was also similar between the 3 arms (Table 14).

TABLE 14

| | Nivolumab + | | Sotigalimab + | | Sotigalimab + | |
|---|---|---|---|---|---|---|
| | Chemo | | Chemo | | Nivolumab + Chemo | |
| | (N = 34) | | (N = 36) | | (N = 35) | |
| Treatment exposure | | | | | | |
| Treatments duration (month), median (range) | 5.2 | (0-19) | 5.1 | (0-20) | 4.7 | (0-24) |
| Chemotherapy treatment cycles, median (range) | 6.0 | (1-23) | 6.0 | (1-22) | 6.0 | (1-25) |
| Patients who received ≥1 dose, n (%) | | | | | | |
| Sotigalimab | 0 | | 34 | (94) | 33 | (94) |
| Nivolumab | 34 | (100) | 0 | | 35 | (100) |
| Gemcitabine | 34 | (100) | 36 | (100) | 35 | (100) |
| nab-Paclitaxel | 34 | (100) | 36 | (100) | 35 | (100) |
| Relative dose intensity, median (IQR), % | | | | | | |
| Sotigalimab | — | | 100 | (81-100) | 100 | (100-100) |
| Nivolumab | 89 | (74-100) | — | | 100 | (83-100) |
| Gemcitabine | 76 | (58-95) | 80 | (64-89) | 68 | (52-88) |
| nab-Paclitaxel | 69 | (52-89) | 71 | (60-84) | 68 | (51-88) |
| Cumulative dose, median (IQR) | | | | | | |
| Sotigalimab, mg/kg | — | | 1.7 | (1.2-2.6) | 1.5 | (1.2-2.4) |
| Nivolumab, mg | 2,280 | (960-3,300) | — | | 2,400 | (960-3,120) |
| Gemcitabine, mg/m² | 14,200 (6,200- | | 14,400 (9,480- | | 11,080 (5,800- | |
| | 18,000) | | 21,320) | | 16,600) | |
| nab-Paclitaxel, mg/m² | 1,388 | (768-2,108) | 1,788 | (1,020-2,444) | 1,385 | (728-2,063) |
| Dose modifications | | | | | | |
| Patients with ≥1 dose reduction, n (%) | 21 | (62) | 25 | (69) | 22 | (63) |
| Sotigalimab, mg/kg | — | | 7 | (19) | 1 | (3) |
| Nivolumab, mg | 0 | | — | | 0 | |
| Gemcitabine, mg/m² | 21 | (62) | 25 | (69) | 22 | (63) |
| nab-Paclitaxel, mg/m² | 25 | (74) | 26 | (72) | 22 | (63) |
| Patients with ≥1 dose not administered, n (%) | | | | | | |
| Sotigalimab, mg/kg | — | | 17 | (47) | 13 | (37) |
| Nivolumab, mg | 22 | (65) | — | | 17 | (49) |
| Gemcitabine, mg/m² | 22 | (65) | 27 | (75) | 25 | (71) |
| nab-Paclitaxel, mg/m² | 23 | (68) | 28 | (78) | 24 | (69) |
| Patient with ≥1 dose interrupted, n (%) | | | | | | |

| | | | | | |
|---|---|---|---|---|---|
| Sotigalimab, mg/kg | — | 21 | (58) | 13 | (37) |

| | | | | | |
|---|---|---|---|---|---|
| Nivolumab, mg | 2 | (6) | — | 2 | (6) |
| Gemcitabine, mg/m² | 1 | (3) | 0 | 2 | (6) |

| | | | | | |
|---|---|---|---|---|---|
| nab-Paclitaxel, mg/m² | 1 | (3) | 2 | (6) | 0 |

Abbreviations: chemo = chemotherapy; IQR = interquartile range; kg = kilogram; m² = meters squared; mg = milligram.
*Includes all randomized and dosed patients in phase 2 and DLT-evaluable patients from phase 1b enrolled at the recommended phase 2 dose of sotigalimab.

### Example 9. Clinical Activity

The primary endpoint was 1-year OS rate versus a historical control rate of 35%14. This study was not powered for comparison between arms.

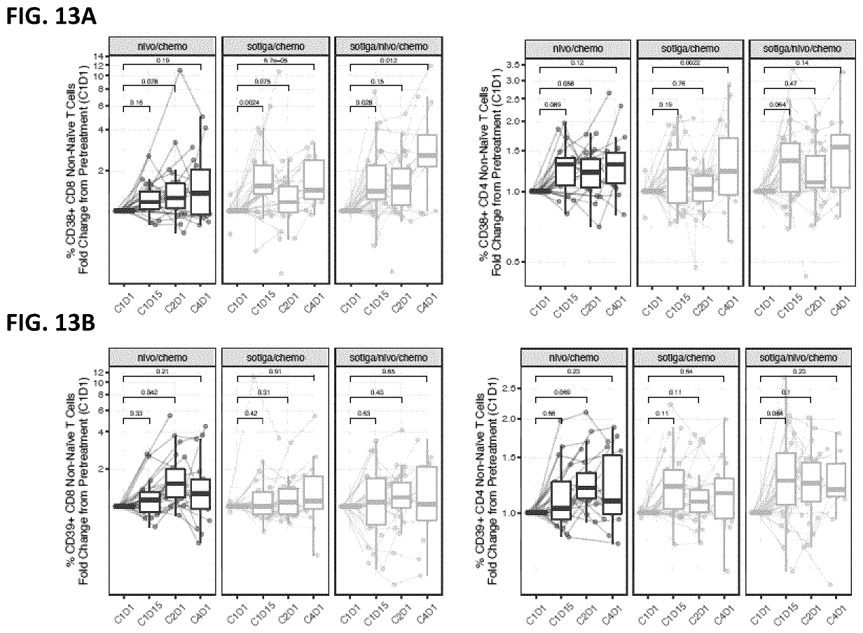

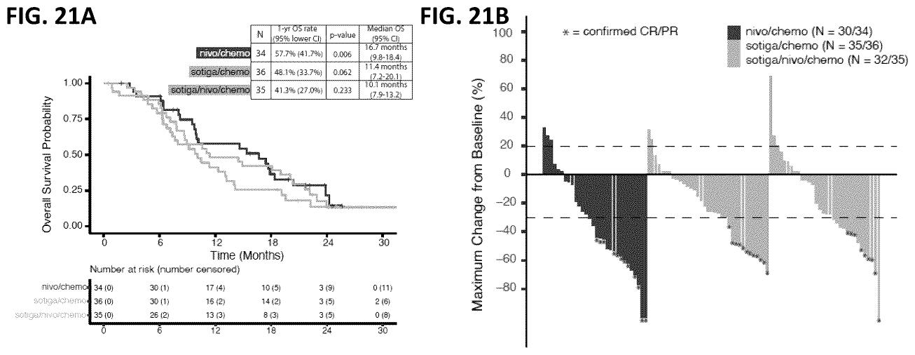

For nivo/chemo, the 1-year OS rate was 57.7% (1-sided p=0.006; 1-sided 95% lower confidence bound=41.7%) and median OS was 16.7 months (95% CI: 9.8-18.4) (FIG. 21 FIG. 1). The median progression-free survival (PFS) was 6.4 months (95% CI: 5.2-8.8), investigator-assessed objective response rate (ORR) was 50.0% (95% CI: 32.4-67.6), disease control rate (DCR) was 73.5% (95% CI: 55.6-87.1), and median duration of response (DOR) was 7.4 months (95% CI: 2.1—not estimable) (FIG. 13, Table 15).

TABLE 15

| | Nivolumab + | | Sotigalimab + | | Sotigalimab + | |
|---|---|---|---|---|---|---|
| | Chemo | | Chemo | | Nivolumab + Chemo | |
| | (N = 34) | | (N = 36) | | (N = 35) | |
| 1-year overall survival | 57.7 | | 48.1 | | 41.3 | |
| p-value vs. 35% from Von Hoff (2013) | 0.006 | | 0.062 | | 0.233 | |
| 1-sided 95% lower confidence bound, % | 41.7 | | 33.7 | | 27.0 | |
| Overall survival, months | | | | | | |
| Median | 16.7 | | 11.4 | | 10.1 | |
| 95% CI | 9.8-18.4 | | 7.2-20.1 | | 7.9-13.2 | |
| Best response, n (%) | | | | | | |
| Complete response | 1 | (3) | 0 | | 0 | |
| Partial response | 16 | (47) | 12 | (33) | 11 | (31) |
| Confirmed partial response, n | 11 | | 12 | | 9 | |
| Unconfirmed partial response, n | 5 | | 0 | | 2 | |
| Stable disease | 8 | (34) | 16 | (44) | 13 | (37) |
| Progressive disease | 5 | (15) | 5 | (14) | 7 | (20) |
| Could not be evaluated^{a} | 4 | (12) | 3 | (8) | 4 | (11) |
| Objective response rate, n (%) | 17 | (50) | 12 | (33) | 11 | (31) |
| 95% CI | 32-68 | | 19-51 | | 17-49 | |
| Disease control rate^{b}, n (%) | 25 | (74) | 28 | (78) | 24 | (69) |
| 95% CI | 56-87 | | 61-90 | | 51-83 | |
| Duration of response, months | | | | | | |
| Median | 7.4 | | 5.6 | | 7.9 | |
| 95% CI | 2.1-NE | | 3.8-8.0 | | 1.9-NE | |
| Progression-free survival, months | | | | | | |

| Median | 6.4 | 7.3 | 6.7 |
|---|---|---|---|
| 95% CI | 8.3-8.8 | 5.4-9.2 | 4.2-9.8 |

Abbreviations: chemo = chemotherapy; CI = confidence interval; NE = not estimable.
* Includes all randomized and dosed patients in phase 2 and DLT-evaluable patients in phase 1b enrolled at the recommended phase 2 dose of sotigalimab.
^{a}Not evaluable included patients who only had one tumor assessment with overall response of Not Evaluable (1 in nivo/chemo) or who did not have any post-baseline tumor assessments due to: initiation of another systemic cancer therapy after treatment discontinuation (1 in nivo/chemo, 2 in sotiga/chemo, 1 in sotiga/nivo/chemo), death (2 in sotiga/nivo/chemo), withdrawal of consent/last to follow-up (1 each in nivo/chemo and sotiga/chemo), or inability due to clinical deterioration (1 each in nivo/chemo and sotiga/chemo).
^{b}Disease control rate is defined as the proportion of patients with a best overall response of complete or partial response or stable disease at least 7 weeks after study drug initiation.

For sotiga/chemo, the 1-year OS rate was 48.1% (1-sided p=0.062; 1-sided 95% lower confidence bound=33.7%) and median OS was 11.4 months (95% CI: 7.2-20.1). The median PFS was 7.3 months (95% CI: 5.4-9.2), investigator-assessed ORR was 33.3% (95% CI: 18.6-51.0), DCR was 77.8% (95% CI: 60.9-89.9), and median DOR was 5.6 months (95% CI: 3.8-8.0).

For sotiga/nivo/chemo, the 1-year OS rate was 41.3% (1-sided p=0.233; lower confidence bound=27.0%) and median OS was 10.1 months (95% CI: 7.9-13.2). The median PFS was 6.7 months (95% CI: 4.2-9.8), investigator-assessed ORR was 31.4% (95% CI: 16.9-49.3), DCR was 68.6% (95% CI: 50.7-83.2), and median DOR was 7.9 months (95% CI: 1.9—not estimable).

### Example 10. Adverse Events

The spectrum, frequency, and severity of treatment-related adverse events (TRAEs) were similar across the arms and consistent with the safety profile observed in Phase Ib¹³. Overall, 106 (98%) patients reported at least one TRAE. The most common nonhematologic TRAEs of any grade were nausea, fatigue, pyrexia, and chills (Table 16).

| Nivolumab + | | Sotigalimab + | | Sotigalimab + | |
|---|---|---|---|---|---|
| Chemo | | Chemo | | Nivolumab + Chemo | |
| (N = 36) | | (N = 37) | | (N = 35) | |
| Any | Grade | Any | Grade | Any | Grade |
| Grade | 3-4 | Grade | 3-4 | Grade | 3-4 |

Treatment-related adverse events with incidence ≥20% in the arm.

| MedDRA ( [symbol] 23.0) Preferred Terms, n (%) | | | | | | | | | | | | |
|---|---|---|---|---|---|---|---|---|---|---|---|---|
| Nausea | 2 [symbol] | ( [symbol] 9) | 0 | | 32 | (87) | 0 | | 2 [symbol] | ( [symbol] 0) | 0 | |
| Fatigue | 2 [symbol] | ( [symbol] 9) | 9 | (25) | 27 | (73) | 5 | (14) | 27 | (77) | [symbol] | (14) |
| Py [symbol] | 11 | (31) | 0 | | 28 | (76) | 1 | ( [symbol] ) | 24 | (69) | 1 | (3) |
| Chills | 3 | (8) | 0 | | 30 | (81) | 3 | ( [symbol] ) | 27 | (77) | 0 | |
| Anemia | 21 | ( [symbol] ) | 12 | ( [symbol] 3) | 20 | (54) | 9 | (24) | 1 [symbol] | (51) | [symbol] | (23) |
| Aspartate amino [symbol] increased | 1 [symbol] | (50) | 7 | (19) | 24 | ( [symbol] ) | 14 | ( [symbol] ) | 20 | ( [symbol] 7) | 9 | (26) |
| A [symbol] amino [symbol] increased | 16 | (44) | 3 | (8) | 2 [symbol] | (54) | 6 | (1 [symbol] ) | 20 | ( [symbol] 7) | 8 | (23) |
| D [symbol] | 19 | ( [symbol] 3) | 0 | | 13 | (35) | 1 | (3) | 16 | (45) | 2 | ( [symbol] ) |
| Vomiting | 11 | (31) | 0 | | 21 | (57) | 1 | (3) | 16 | (45) | 1 | (3) |
| Decreased appetite | 17 | (47) | 0 | | 19 | (51) | 0 | | 9 | (2 [symbol] ) | 0 | |
| Al [symbol] | 14 | (39) | 0 | | 17 | (4 [symbol] ) | 1 | (3) | 12 | (34) | 0 | |
| Edema per [symbol] | 13 | (36) | 0 | | 17 | (4 [symbol] ) | 1 | (3) | 12 | (34) | 0 | |
| P [symbol] count decreased | 12 | (33) | 3 | (8) | 13 | (35) | [symbol] | ( [symbol] ) | 17 | (49) | 4 | (11) |
| Neutrophil count decreased | 13 | (36) | 7 | (19) | 1 [symbol] | (3 [symbol] ) | 13 | (38) | 14 | (40) | 8 | (23) |
| Rash | 17 | (47) | 4 | (11) | [symbol] | (24) | 0 | | 13 | (37) | 0 | |
| Neuropathy perp [symbol] | 10 | (2 [symbol] ) | 1 | (3) | 1 [symbol] | (43) | 1 | (3) | 9 | (2 [symbol] ) | 0 | |
| Throm [symbol] | [symbol] | (25) | 1 | (3) | 11 | (30) | 4 | (11) | 14 | (40) | 2 | ( [symbol] ) |
| [symbol] | 3 | (8) | 1 | (3) | 15 | (41) | 0 | | 1 [symbol] | (37) | 0 | |
| Blood [symbol] phosph [symbol] increased | 7 | (1 [symbol] ) | 1 | ( [symbol] ) | 1 [symbol] | (32) | 2 | ( [symbol] ) | 11 | (31) | 3 | (9) |
| Dy [symbol] | 9 | (25) | [symbol] | ( [symbol] ) | [symbol] | (22) | 2 | ( [symbol] ) | 9 | (2 [symbol] ) | 2 | ( [symbol] ) |
| N [symbol] | 7 | (19) | 5 | (14) | 12 | (32) | 10 | (27) | 6 | (17) | 5 | (14) |
| White blood cell count decreased | 10 | (2 [symbol] ) | 5 | (14) | 7 | (19) | 3 | (8) | 6 | (17) | 3 | (9) |
| Cyto [symbol] re [symbol] syndrome | 0 | | 0 | | 9 | (24) | 3 | (8) | 12 | (34) | 2 | ( [symbol] ) |
| Lymphocyte count decreased | [symbol] | (17) | [symbol] | (17) | [symbol] | (24) | 6 | (16) | 6 | (17) | 5 | (14) |
| Peripheral sensory neur [symbol] | [symbol] | (22) | 0 | | 7 | (19) | 2 | ( [symbol] ) | 6 | (17) | 0 | |
| Hypotension | 2 | ( [symbol] ) | 0 | | 9 | (24) | 0 | | 9 | (26) | 0 | |
| Myalgia | [symbol] | ( [symbol] ) | 0 | | 5 | (22) | 0 | | 8 | (23) | 1 | (3) |
| Cough | 4 | (11) | 0 | | 4 | (11) | 0 | | 8 | (23) | 0 | |
| Headache | [symbol] | (3) | 0 | | 10 | (27) | 0 | | 4 | (11) | 0 | |
| Dysgeusia | 4 | (11) | 0 | | 2 | ( [symbol] ) | 0 | | 7 | (20) | 0 | |
| My [symbol] | 2 | (6) | 1 | ( [symbol] ) | 8 | (22) | [symbol] | (3) | 2 | (6) | 1 | (3) |

| | | | | | | | | |
|---|---|---|---|---|---|---|---|---|
| U [symbol] | 0 | 0 | 4 | (11) | 0 | 8 | (23) | 0 |

Counts when combining similar preferred terms:

| Combined Terms, n (%) | | | | | | | | | | | | |
|---|---|---|---|---|---|---|---|---|---|---|---|---|
| Neuropathy peripheral, Peripheral motor | 19 | (53) | 1 | (2) | 23 | ( [symbol] 2) | 4 | (11) | 14 | (40) | 0 | |
| neuropathy, Peripheral sensory neuropathy | | | | | | | | | | | | |
| Neutrop [symbol] , Neutro [symbol] count decreased, | 19 | (53) | 12 | ( [symbol] ) | 22 | ( [symbol] ) | 20 | (54) | 20 | (57) | 1 [symbol] | ( [symbol] 7) |
| White blood cell count decreased | | | | | | | | | | | | |
| Plat [symbol] count decreased, Thro [symbol] | 17 | (47) | 4 | (11) | 21 | (57) | 6 | (16) | 21 | ( [symbol] 0) | 6 | (17) |

Abbreviations: chemo = chemotherapy; MedDRA = Medical Dictionary for Regulatory Activites.
* Includes all Phase 1b and Phase 2 patients who received at least 1 dose of any study drug. For safety analyses, patients were grouped according to the study treatment actually received. [symbol]
indicates data missing or illegible when filed

The most common grade 3-4 TRAEs were hematologic and generally transient in nature. Adverse events of special interest (AESI), including cytokine release syndrome (CRS), infusion reactions, thrombocytopenia, and elevated liver function tests (LFTs), were observed in 90 (83%) patients (Table 17).

TABLE 17

| | Nivolumab + | | Sotigalimab + | | Sotigalimab + | |
|---|---|---|---|---|---|---|
| | Chemo | | Chemo | | Nivolumab + Chemo | |
| | (N = 36) | | (N = 37) | | (N = 35) | |
| Patients with at least one AESI, n (%) | 28 | (78) | 32 | (87) | 30 | (86) |
| Cytokine release syndrome | 0 | | 9 | (24) | 12 | (34) |
| Grade 1 | 0 | | 0 | | 1 | (3) |
| Grade 2 | 0 | | 6 | (16) | 9 | (26) |
| Grade 3 | 0 | | 3 | (8) | 2 | (6) |
| Increased liver function text results | 24 | (67) | 30 | (81) | 26 | (74) |
| Grade 1 | 5 | (14) | 1 | (3) | 5 | (14) |
| Grade 2 | 7 | (19) | 11 | (30) | 6 | (17) |
| Grade 3 | 12 | (33) | 18 | (49) | 14 | (40) |
| Grade 4 | 0 | | 0 | | 1 | (3) |
| Infusion related reaction | 2 | (6) | 5 | (14) | 5 | (14) |
| Grade 1 | 0 | | 2 | (5) | 0 | |
| Grade 2 | 1 | (3) | 2 | (5) | 5 | (14) |
| Grade 3 | 1 | (3) | 1 | (3) | 0 | |
| Low platelet count | 18 | (50) | 21 | (57) | 22 | (63) |
| Grade 1 | 5 | (14) | 7 | (19) | 5 | (14) |
| Grade 2 | 8 | (22) | 8 | (22) | 10 | (29) |
| Grade 3 | 3 | (8) | 4 | (11) | 6 | (17) |
| Grade 4 | 2 | (6) | 2 | (5) | 1 | (3) |

Abbreviations: AESI = adverse event of special interest; chemo = chemotherapy.
* Includes all Phase 1b and Phase 2 patients who received at least 1 dose or any study drug. For safety analyses, patents were grouped according to the study treatment actually received.
Adverse events were graded according to the National Cancer Institute Common Terminology Criteria for Adverse Events (NCI CTCAE), version 4.0 [symbol] . As a limitation, due to overlapping characteristics, there is potential for variability in assessment between terms (e.g., infusion related reaction and cytokines release syndrome), which may lead to under or over-representation of incidence of specific terms.
Cytokine release syndrome is defined as an adverse event with a MedDRA Preferred Term matching ‘Cytokine release syndrome’, regardless of seriousness, severity or relationship to study drugs.
Increased liver function test results in defined as an adverse event with a MedDRA Preferred Term matching ‘Alanine aminotransferase increased’, ‘Aspartate aminotransferase increased’, ‘Blood alkaline phosphatase increased’, ‘Blood bilirubin increased’, ‘Hepatic enzyme increased’ or ‘Hyperbilirubinemia’, regardless of seriousness, severity or relationship to study drugs.
Infusion related reaction is defined as an adverse event with a MedDRA Preferred Term matching ‘Infussion related reaction’, regardless of seriousness, severity or relationship to study drug.
Low platelet count is defined as an adverse event with a MedDRA Preferred Term matching ‘Platelet count decreased’ or ‘Thrombocytopenia’, regardless of seriousness, severity or relationship to study drugs. [symbol]
indicates data missing or illegible when filed

CRS was observed in 0, 9 (24%), and 12 (34%) patients in nivo/chemo, sotiga/chemo, and sotiga/nivo/chemo, respectively, with 5 events assessed as grade 3 (3 in sotiga/chemo and 2 in sotiga/nivo/chemo). Grade 4 or 5 CRS was not observed. Infusion related reactions were observed in 2 (6%), 5 (14%), and 5 (14%) patients, respectively. Low platelet count occurred in 18 (50%), 21 (57%), and 22 (63%) patients, respectively. Elevated LFTs were observed in 24 (67%), 30 (81%), and 26 (74%) patients, respectively. Six (17%) patients on nivo/chemo, 1 (3%) on sotiga/chemo, and 1 (3%) on sotiga/nivo/chemo discontinued all study drugs due to an AE (Table 18).

TABLE 18

| | Nivolumab + | | Sotigalimab + | Sotigalimab + |
|---|---|---|---|---|
| | Chemo | | Chemo | Nivolumab + Chemo |
| | (N = 36) | | (N = 37) | (N = 35) |
| Treatment discontinuation due to an AE, n (%) | 6 | (17) | 1 (3) | 1 (3) |
| Thrombotic microangiopathy | 2 | (6) | 0 | 0 |
| Pneumonitis | 1 | (3) | 1 (3) | 0 |
| Hyperbilirubinemia | 1 | (3) | | 0 |
| Myocarditis | 1 | (3) | | 0 |
| Neuropathy peripheral | 1 | (3) | | 0 |

| | | |
|---|---|---|
| Pyrexia | 0 | 1 (3) |

Abbreviations: AE = adverse event; chemo = chemotherapy.
* Includes all Phase 1b and Phase 2 patients who received at least 1 dose of any study drug. For safety analyses, patients were grouped according to the study treatment actually received.

Two patients died due to an adverse event: acute hepatic failure on sotiga/chemo (causality could not be determined so considered possibly related to all study drugs) and intracranial hemorrhage on sotiga/nivo/chemo (again, possibly related to all study drugs).

### Example 11. Pharmacodynamic Effects

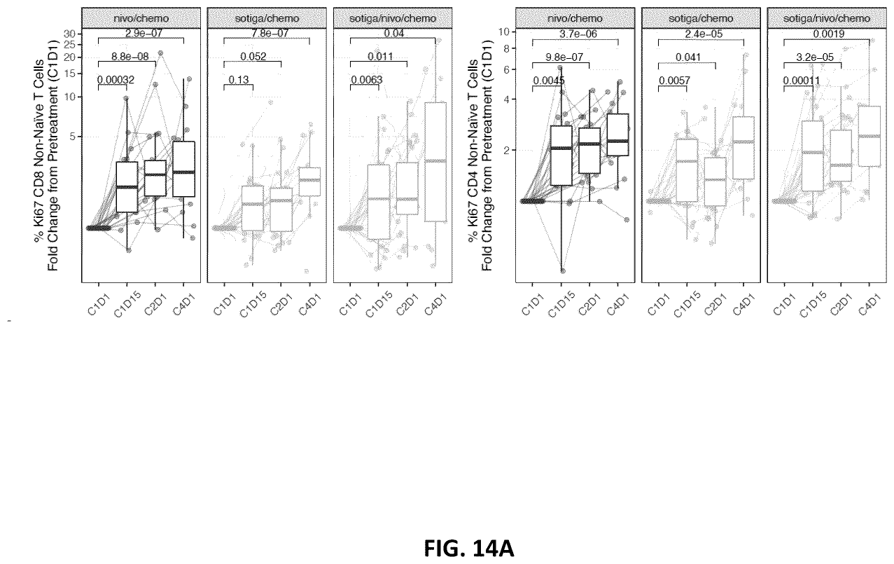

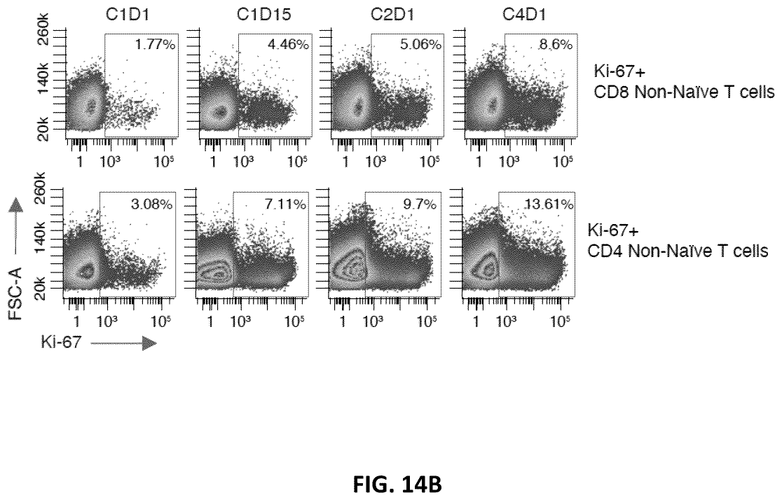

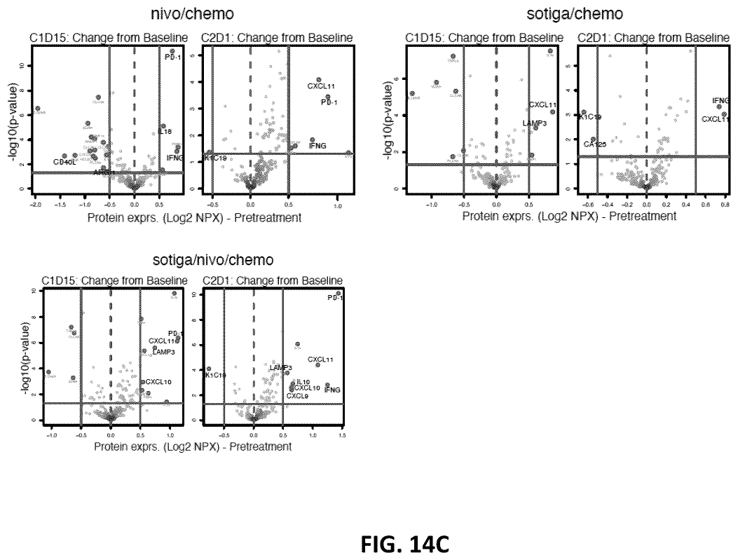

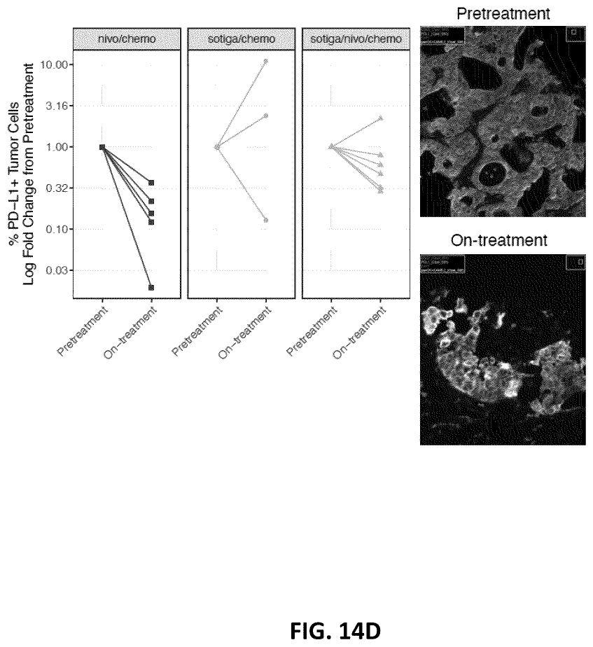

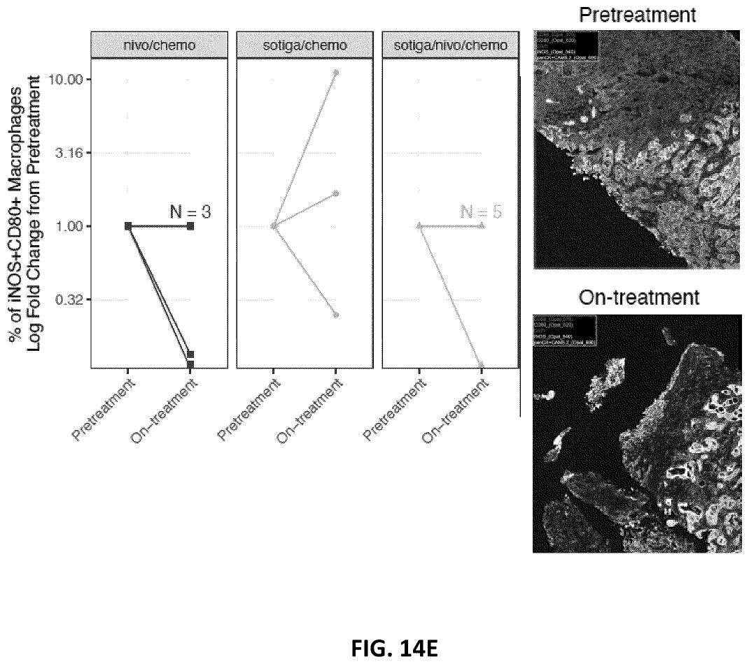

To understand pharmacodynamic effects in each arm, multi-omic profiling of serial patient blood samples and tumor biopsies obtained pre-treatment and on-treatment was performed. In all three arms, longitudinal profiling (See Methods) of patient peripheral blood mononuclear cells (PBMCs) revealed increases in proliferating (Ki-67+) non-naïve (Table 19; Immune Cell Populations Defined) CD8 and CD4 T cells on-treatment (FIGS. 14A-14B).

TABLE 19

| Immune Cell Population | Markers used to Define Population |
|---|---|
| Non-naïve T cells | (CD45RA−CD27+) (CD45RA−CD27−), (CD45RA+CD27−) |
| Effector Memory T cells | CD45RA−CCR7− |
| Effector Memory 1 (EM1) T cells | CD45RA−CD27+CCR7− |
| Central Memory T cells | CD45RA−CD27+CCR7+ |
| Conventional Dendritic Cells | HLA−DR+CD14−CD16−CD11c+ |
| Cross Presenting Dendritic Cells | HLA−DR+CD14−CD16−CD11c+CD141+ |
| B cells | CD19+ |
| Monocytic Myeloid Derived Suppressor Cells | CD14+CD16−HLA−DRlo |

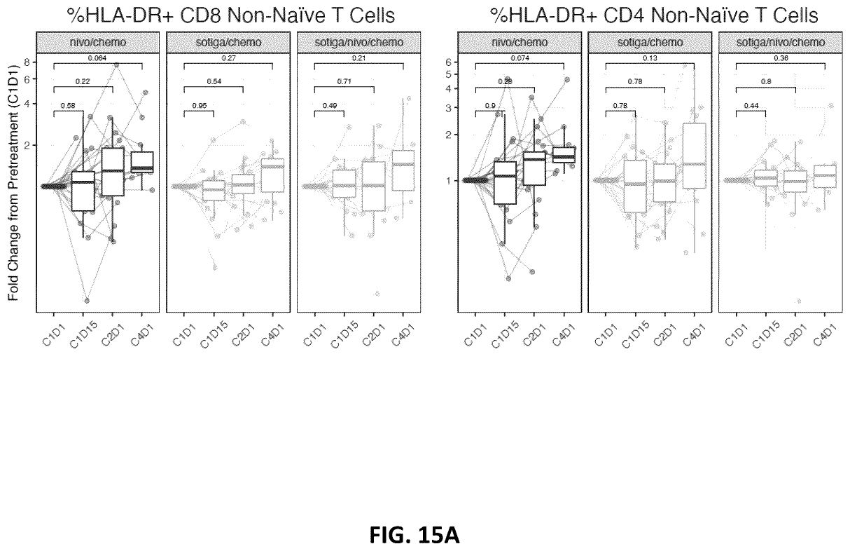

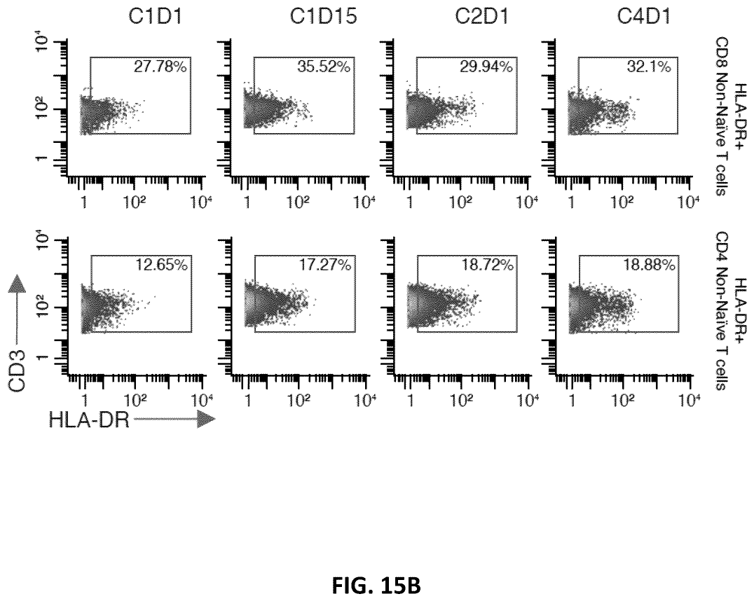

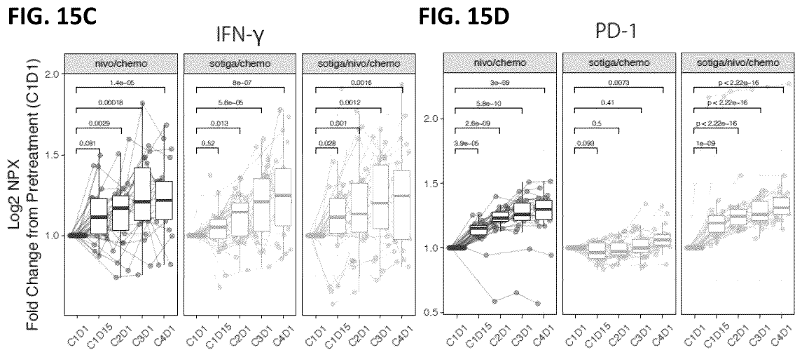

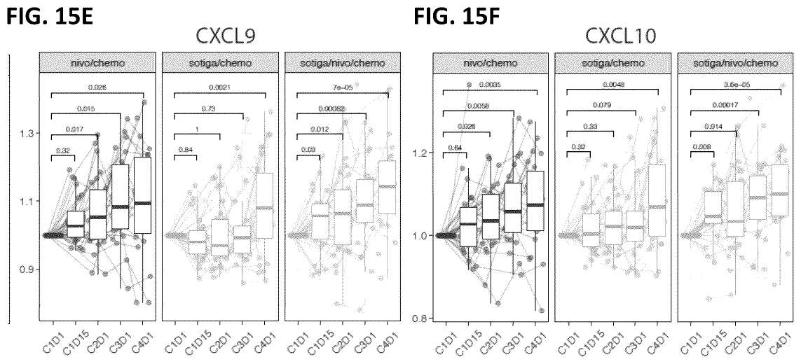

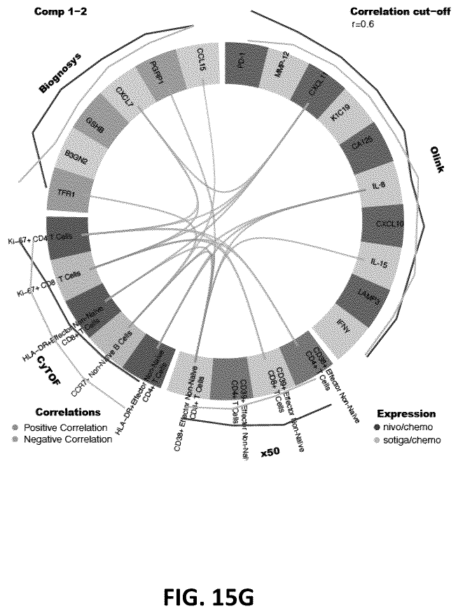

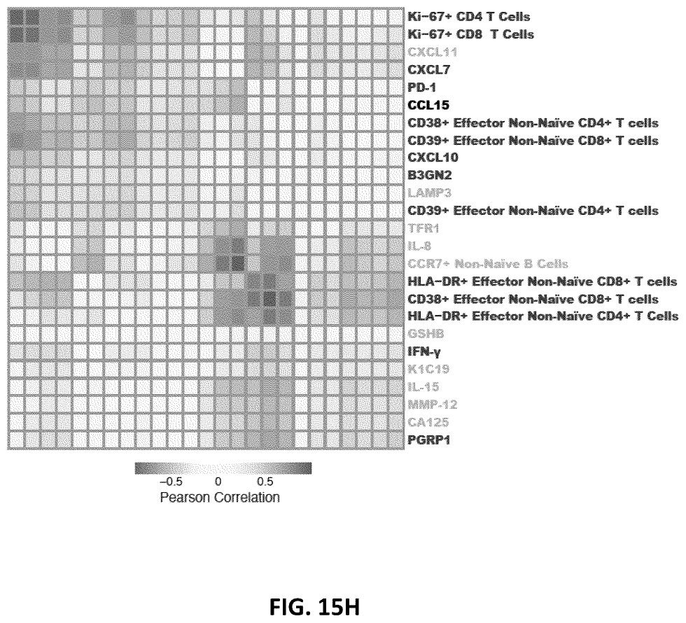

This increase was strongest and observed earlier in the nivo/chemo arm and to a lesser extent the sotiga/chemo arm; comparatively, the effect was numerically diminished in the sotiga/nivo/chemo arm, possibly indicating lessened systemic immune activation of T cell expansion, or altered kinetics of T cell modulation that were not captured in the time-series analysis. Circulating activated (HLA-DR+, CD38+ or CD39+) non-naïve CD4 and CD8 T cells also increased in all three arms, especially in nivo containing arms (FIG. 15A FIG. 15B, and FIG. 13). Among an array of 172 serum proteins studied, patients in all three treatment arms had on-treatment decreases in known biomarkers prognostic to pancreatic cancer, such as K1C19 (pancreatic ductal protein), that tracked with tumor regression measurements (FIG. 14C and Table 20).

TABLE 20

| | | | Significant | | Directional |
|---|---|---|---|---|---|
| | | | On-treatment | | Change from |
| Classification | Feature | Sample | Timepoint(s) | P-Value | Pretreatment |
| Immunosuppression | PD−1 | Serum | C1D15, | [symbol] .09E−12 | Increases |
| | | | C2D1, C3D1 | 0.0003 [symbol] , 3.84E−0 [symbol] | |
| | Arginase-1 ARG1 | Serum | C1D15 | 0.01 [symbol] 69 | Decreases |
| | MMP-12 | Serum | C3D1 | 0.00124 | Decreases |
| | HLA−Drlo Ki−67+ mMDSC | | C1D15 | 9.27E−06 | Increases |
| | (% of Classical Monocytes) | | | | |
| | CTLA-4+ Non-Naïve CD8 T cells | PBMCs | C2D1 | 0.00364 | Increases |
| | (% of Non-Naïve CD8 T cells) | | | | |
| | CP−L1+ Non-Naïve CD8 T cells | PBMCs | C2D1 | 0.03229 | Increases |
| | (% of Non-Naïve CD8 T cells) | | | | |
| | PD−L1+ T cells (% of T cells) | PBMCs | C2D1 | 0.04444 | Increases |
| | PD−L1+ Non-Naïve CD8 T cells | PBMCs | C4D1 | 0.00012 | Decreases |
| | (% of Non-Naïve CD8 T cells) | | | | |
| | PD−L1+ T cells (% of T cells) | PBMCs | C4D1 | 0.00605 | Decreases |
| | PD−L1+ Non-Naïve CD8 T cells | PBMCs | C1D15 | 0.03137 | Decreases |
| | (% of Non-Naïve CD8 T cells) | | | | |
| | CD11b+ B cells (% of B cells) | PBMCs | C1D15, C4D1 | 0.01216, 0.00942 | Decreases, Increases |
| | CCR7+ CD11b+ B cells | PBMCs | C1D15 | 0.01700 | Decreases |
| | (% of B cells) | | | | |
| Pattern Recognition | CLEC-4A | Serum | C1D15 | 3.47E−08 | Decreases |
| Receptors | CLEC-6A | Serum | C1D15 | 0.00017 | Decreases |
| | CLEC-4D | Serum | C1D15 | 0.00017 | Decreases |
| | CLEC-7A | Serum | C3D1 | 2.51E−06 | Increases |
| Chemokine | CXCL5 | Serum | C1D15 | 0.000 [symbol] | Decreases |
| Activation & | IFN-γ | Serum | C1D15, | 0.00091, | Increases |
| Proliferation of T | | | C2E1, C3D1 | 0.014 [symbol] , 9.73E−05 | Decreases |
| cells | IL-7 | Serum | C1D15 | 0.0017 [symbol] | Increases |
| | CD40L | Serum | C1D15 | 0.0021 | Decreases |
| | IL-18 | Serum | C1D15 | 7.79E−06 | Increases |
| | IL-8 | Serum | C3D1 | 7.77E−05 | Decreases |
| | Ki−67+ T cells (% of T cells) | PBMCs | C1D15 | 3.93E−05 | Increases |
| | Ki−67+ CD8 T cells | PBMCs | C1D15 | 0.00087 | Increases |
| | (% of CD8 T cells) | | | | |
| | Ki−67+ Non-Naïve CD8 T cells | PBMCs | C2D1 | 0.00364 | Increases |
| | (% of Non-Naïve CD8 T cells) | | | | |
| | CD38+ Non-Naïve CD8 T cells | PBMCs | C4D1 | 0.00445 | Increases |
| | (% of Non-Naïve CD8 T cells) | | | | |

[symbol]

indicates data missing or illegible when filed

Patients in all three treatment arms also had decreases in the immunosuppressive molecules IL-8 and MMP-12 (Table 20, Table 21,), whereas patients treated with nivo/chemo exhibited early (C1D15) decreased levels of the immunosuppressive protein arginase 1 and the co-stimulatory ligand CD40L (FIG. 14C).

TABLE 21

| | | | Significant | | Directional |
|---|---|---|---|---|---|
| | | | On-treatment | | Change from |
| Classification | Feature | Sample | Timepoint(s) | P-Value | Pretreatment |
| Activation & | IL-18 | Serum | C1D15 | 2.9974E−0 [symbol] | Increases |
| Proliferation of T | IFN-γ | Serum | C2D1, C3D1 | 0.00046, 0.00059 | Increases |
| cells | IL-15 | Serum | C3D1 | 4. [symbol] 0 [symbol] E−08 | Increases |
| | Ki−67+ Non-Naïve CD8 T cells | PBMCs | C4D1 | 0.00452 | Increases |
| | (% Non-Naïve CD8 T cells) | | | | |
| | Ki−67+ T cells (% of T cells) | PBMCs | C4D1 | 0.00873 | Increases |
| Type 1 Immunity | IL-12A | Serum | C1D15 | [symbol] .2 [symbol] 4E−0 [symbol] | Decreases |
| PDAC Prognostic | Keratin-19 - KIC19 | Serum | C2D1, C3D1 | 0.00077, 0.00033 | Decreases |
| Factors | Mucin-16 - MUC18 | Serum | C2D1, C3D1 | 0.009 [symbol] , 0.00 [symbol] | Decreases |
| | IL-8 | Serum | C3D1 | 0.01683 | Decreases |
| Pattern Recognition | CLEC-4C | Serum | C3D1 | 1.47829E−07 | Increases |
| Receptors | CLEC-4D | Serum | C1D15 | 0.0084 [symbol] | Decreases |
| Monocytes | Intermediates Monocytes (CD16+CD14+) | PBMCs | C1D15 | 0.00497 | Decreases |
| | (% of HLA−DR+ cells) | | | | |
| | Non-classical Monocytes | PBMCs | C2D1 | 0.03535 | Decreases |
| | (% of HLA−DR+ cells) | | | | |
| Immunosuppression | MMP-12 | Serum | C3D1 | 0.0101 [symbol] | Decreases |
| | PD−1+ T cells (% of T cells) | PBMCs | C1D15 | 0.00253 | Decreases |
| | Ki−67+ Tregs (% of Tregs) | PBMCs | C1D15 | 0.04190 | Increases |
| | PD−L1+ Tregs (% or Tregs) | PBMCs | C1D15 | 0.04 [symbol] 0 | Increases |
| | CD38+ Tregs (% or Tregs) | PBMCs | C1D15 | 0.0480 | Increases |
| | PD−L1+ T cells (% of T cells) | PBMCs | C1D15 | 0.0489 | Increases |
| | T [symbol] + Tregs (% of Tregs) | PBMCs | C1D15 | 0.00 [symbol] 11 | Decreases |
| | PD−1+ Non-Naïve CD8 T cells | PBMCs | C2D1 | 0.00115 | Decreases |
| | (% Non-Naïve CD8 T cells) | | | | |
| | HLA−DRlo mMDSC | PBMCs | C1D15 | 0.007 [symbol] | Increases |
| | (% of Classical Monocytes) | | | | |
| T cell Immunity | Non-Naïve CD8 T cells | PBMCs | C1D15, C4D1 | 0.00488, 0.0020 [symbol] | Decreases |
| | (% of CD8 T cells) | | | | |
| B cell Biology | CCR7+ B cells (% of B cells) | PBMCs | C2D1, C5D1 | 0.01000, 0.01645 | Decreases |
| | HLA−DR+ Plasmablasts | PBMCs | C2D1 | 0.01094 | Decreases |
| | (% of Plasmablasts) | | | | |
| | CCR7+ Plasmablasts | PBMCs | C2D1 | 0.0119 [symbol] | Decreases |
| | (% of Plasmablasts) | | | | |
| | CD40+ Memory B cells | PBMCs | C2D1 | 0.0286 | Decreases |
| | (% or Memory B cells) | | | | |
| | CD40+ B cells (% of B cells) | PBMCs | C2D1 | 0.0278 [symbol] 1 | Decreases |
| | CD40+CD27+ B cells | PBMCs | C3D1 | 0.0 [symbol] 34 | Decreases |
| | (% of CD27+ B cells) | | | | |
| | CCR7+ Memory B cells | PBMCs | C4D1 | 0.00312 | Decreases |
| | (% of Memory B cells) | | | | |
| | CD27+ B cells (% of B cells) | PBMCs | C1D15 | 0.0 [symbol] 93 | Decreases |
| | B cells (% of CD45+ cells) | PBMCs | C4D1 | 0.02 [symbol] 1 | Decreases |
| | CCR7+ Memory B cells | PBMCs | C2D1 | 0.00578 | Decreases |
| | (% of Memory B cells) | | | | |
| Antigen Experienced | PD−1+CD38+Non-Naïve CD4 T cells | PBMCs | C4D1 | 0.036 [symbol] 4 | Increases |
| T cells | (% Non-Naïve CD4 T cells) | | | | |
| Dendritic Cell | 141+ Dendritic Cells | PBMCs | C4D1 | 0.0434 [symbol] | Decreases |
| Biology | (% or Conventional Dendritic Cells) | | | | |
| | 141− Dendritic Cells | PBMCs | C4D1 | 0.04243 | Increases |
| | (% or Conventional Dendritic Cells) | | | | |
| | CD1C+ CD141+ Dendritic Cells | PBMCs | C1D15 | 0.00056 | Increases |
| | (% of 141+ Dendritic Cells) | | | | |
| | CD1C− CD141+ Dendritic Cells | PBMCs | C1D15 | 0.00117 | Decreases |
| | (% of 141+ Dendritic Cells) | | | | |
| | Conventional Dendritic Cells | PBMCs | C [symbol] D1 | 0.04258 | Decreases |
| | (% of HLA−CD+ cells) | | | | |
| | LAMP3 | Serum | C1D15, C3D1 | 0.00049, 0.00011 | Increases |
| | CXCL11 | Serum | C1D15, C3D1 | 0.00007, 0.00042 | Increases |

[symbol]

indicates data missing or illegible when filed

Patients treated with nivo/chemo had early (C1D15) increases in cytokines related to T cell activation and type 1 immunity, most notably soluble PD-1, type-1 skewing chemokines (CXCL9, CXCL10), type-II interferons, and IL-18 (FIG. 14C, Table 20). In contrast, patients treated with sotiga/chemo exhibited early increases (C1D15) in proteins associated with dendritic cell maturation and activation such as LAMP3 and CXCL11 (FIG. 14C, Table 21) followed by later (C1D15, C3D1) upregulation of proteins associated with T cell activation such as type-II interferons and IL-15 (FIG. 14C, FIG. 15C, FIG. 15CD, FIG. 15E, FIG. 15F and Table 21). Increases at C3D1 in IL-15 were uniquely observed in the sotiga/chemo treatment (Table 21). All treatment associated changes in circulating proteins with sotiga/nivo/chemo were also observed to change in the individual nivo/chemo or sotiga/chemo arms, though with different kinetics. For example, patients treated with sotiga/nivo/chemo had earlier (C1D15) increases in LAMP3, CXCL11, CXCL10 and soluble PD-1 at C1D15, and later (C2D1) increases in type-11 interferons and CXCL9 (FIG. 14C, FIG. 15C, FIG. 15CD, FIG. 15E, FIG. 15F, and Table 22).

TABLE 22

| | | | Significant | | Directional |
|---|---|---|---|---|---|
| | | | On-treatment | | Change from |
| Classification | Feature | Sample | Timepoint(s) | P-Value | Pretreatment |
| Activation & | IL-18 | Serum | C1D15, C2D1, | 1.49E−10, 8.41E−07, | Increases |
| Proliferation of T | | | C3D1 | 1.43E−05 | |
| cells | IL-15 | Serum | C3D1 | 1.01E−05 | Increases |
| | IFN-γ | Serum | C2D1, C3D1 | 0.00153, 0.00083 | Increases |
| | CD28 | Serum | C3D1 | 2.82E−06 | Increases |
| | Ki−67+ T cells (% of T cells) | PBMCs | C1D15 | 0.00180 | Increases |
| | Ki−67+ Non-Naïve CD4 T cells | PBMCs | C1D15 | 0.03024 | Increases |
| | (% of Non-Naïve CD4 T cells) | | | | |
| | CD38+ Non-Naïve CD8 T cells | PBMCs | C1D15 | 0.00274 | Increases |
| | (% of Non-Naïve CD8 T cells) | | | | |
| | CD38+ T cells (% of T cells) | PBMCs | C1D15 | 0.00532 | Increases |
| PDAC Prognostic | IL-8 | Serum | C3D1 | 0.04527 | Deceases |
| Factors | Keratin-19 -KIC19 | Serum | C2D1, C3D1 | 7. [symbol] 5E−05, 0.00657 | Deceases |
| | Mucis-16 - MUC16 | Serum | C3D1 | 0.00703 | Deceases |
| Co-stimulation/Th2 | OX40L | Serum | C1D15, C3D1 | 1.40E−08, [symbol] .71E−10 | Increases |
| polarization | | | | | |
| Pattern Recognition | CLEC-4A | Serum | C1D15, C3D1 | 1.78E−07 | Decreases |
| Receptors | CLEC-4C | Serum | C3D1 | 5.82E−07 | Increases |
| | CLEC-7A | Serum | C3D1 | 9.02E−06 | Increases |
| Immunosuppression | PD-1 | Serum | C1D15, C2D1, | 4.24E−07, 7.63E−11, | Increases |
| | | | C3D1 | 6.73E−10 | |
| | IL-10 | Serum | C2D1, C3D1 | 0.00064, 5. [symbol] E−05 | Increases |
| | HLA−DRloKi−67+ mMDSC | PBMCs | C4D1 | 0.02523 | Deceases |
| | (% of Classical Monocytes | | | | |
| | CTLA-4 Non-Naïve CD8 T cells | PBMCs | C4D1 | 0.04805 | Increases |
| | (% of Non-Naïve CD8 T cells) | | | | |
| | CD18+ Tregs (% of Tregs) | PBMCs | C4D1 | 0.02451 | Increases |
| | CTLA-4+ T cells (% of T cells) | PBMCs | C4D1 | 0.01566 | Increases |
| | PD−1+ Non−Naïve CD8 T cells | PBMCs | C1D15, C2D1 | 0.00144, 0.00858 | Deceases |
| | (% of Non−Naïve CD8 T cells) | | | | |
| | PD−1+ Non−Naïve CD4 T cells | PBMCs | C1D15, C2D1 | 0.0114, 0.03641 | Increases |
| | (% of Non−Naïve CD4 T cells) | | | | |
| | CD18+ Tregs (% of Tregs) | PBMCs | C1D15 | [symbol] .45E−05 | Increases |
| | Ki−67+ Tregs (% of Tregs) | PBMCs | C1D15 | 0.0003 [symbol] | Increases |
| | PD−1+ Tregs (% of Tregs) | Serum | C1D15 | 4. [symbol] E−07 | Deceases |
| Type 1 Immunity | IL12A | Serum | C1D15 | 0.00019 | Deceases |
| | CXCL10 | Serum | C1D15, C1D1, | 0.00113, 0.00250, | Increases |
| | | | C3D1 | 4.20E−0 [symbol] | |
| | CXCL9 | Serum | C2D1, C3D1 | 0.00396, 0.00021 | Increases |
| Chemokine | CCL19 | Serum | C3D1 | 7.57E−0 [symbol] | Increases |
| Inflammation | TNF-α | Serum | C3D1 | 4.40E-05 | Increases |
| B cell Biology | B cell (% of CD45+ cells) | PBMCs | C1D15 | 8.77E-07 | Deceases |
| | CD40+ B cells (% of B cells) | PBMCs | C1D15 | 0.01670 | Deceases |
| | B cells (% of CD45+ cells) | PBMCs | C1D15, C4D1 | 8.77E−07, 0.02523 | Deceases |
| Dendritic Cell Biology | Plasmacytoid Dendritic Cells | PBMCs | C1D15 | 0.00138 | Deceases |
| | (% of HLA−DR+) | | | | |
| | CD33+ Plasmacytoid Dendritic Cells | PBMCs | C1D15, C4D1 | 0.01875, 0.0180 [symbol] | Increases |
| | (% of Plasmacytoid Dendritic Cells) | | | | |
| | 141+ Dendritic Cells | PBMCs | C1D15 | 0.01240 | Deceases |
| | (% or Conventional Dendritic Cells) | | | | |
| | 141− Dendritic Cells | PBMCs | C1D15 | 0.01308 | Increases |
| | (% or Conventional Dendritic Cells) | | | | |
| | CD1C+ CD141+ Dendritic Cells | PBMCs | C1D15 | 0.03694 | Increases |
| | (% of 141+ Dendritic Cells) | | | | |
| | CD33+ Conventional Dendritic Cells | PBMCs | C2D1 | 0.04288 | Increases |
| | (% of Conventional Dendritic Cells) | | | | |
| | CXCL11 | Serum | C1D15, C2D1, | 7.66E−07, 3.99E−0 [symbol] , | Increases |
| | | | C3D1 | 1.2 [symbol] #−0 [symbol] | |
| | LAMP3 | Serum | C1D15, C2D1, | 2.47E−0 [symbol] , 0.00017, | Increases |
| | | | C3D1 | 4.2 [symbol] E−07 | |

[symbol]

indicates data missing or illegible when filed

Patient sera were then profiled using unbiased mass spectrometry combined with sparse PLS discriminant analysis to identify critical circulating proteins not identified in the targeted approach (FIG. 14C). Patients treated with nivo/chemo had increases in proteins associated with immune cell migration and T cell activation (GKN1, B3GN2, and PGRP1), and decreases in the chemokine, CXCL7, compared to pretreatment levels (Table 23).

TABLE 23

| | | | | Median | Directional |
|---|---|---|---|---|---|
| | | | Contribution | Importance | Change from |
| Relevant Biology | Factors | Sample | Timepoint(s) | Score | Pretreatment |
| Reported to be an antagonist | Alpha 2-HS Glycoprotein - AHSG | Serum | C3D1 | −0.05704 | Increases |
| of TGPB | | | | | |
| Reported to Downregulate | Apolipoprotein A2 - APOA2 | Serum | C1D15 | 0.18047 | Decreases |
| neutrophil functions by | | | | | |
| decreasing IL- [symbol] production of | | | | | |
| neutrophils | | | | | |
| Upregulated in T cell | Beta-1,3-N- | Serum | C3D1 | −0.19020 | Increases |
| activation, proposed to have a | acetyglucosaminyltransferase- | | | | |
| role in PD1 glycosylation | B3GNT2 | | | | |
| Has been shown to be | | | | | |
| Inversely correlated | | | | | |
| with response to anti- | | | | | |
| PD1 therapy | | | | | |
| Has been reported to be highly | Cellular Communication Network | Serum | C1D15 | 0.17631 | Increases |
| expressed in PDAC | Factor 2 - CCN2 | | | | |
| microenvironment and | | | | | |
| associated with disease | | | | | |
| progression | | | | | |
| Associated with poor | Complement Factor B - CFB | Serum | C1D15 | 0.17716 | Increases |
| prognosis in pancreatic cancer | | | | | |
| Mediates adhesive interactions | Intercellular Adhesion Molecule 2 - | Serum | C3D1 | −0.022818 | Increases |
| important for antigen-specific | ICAM2 | | | | |
| immune response | | | | | |
| Reported to be an agonist of | Platelet Factor 4/CXCL4 - PF4 | Serum | C1D15 | 0.02907 | Increases |
| CCR1 and drives human | | | | | |
| monocyte migration | | | | | |
| Displays antiangiogenic | Platelet Factor 4 Variant 1/CXCL4V1 - | Serum | C2D1 | −0.10450 | Decreases |
| function and it regulated by | PF4V1 | | | | |
| chemokine (C-X-C motif) | | | | | |
| receptor 3 | | | | | |
| Potent chemo [symbol] and | Pro-Platelet Basic Protein/CXCL7- | Serum | C2D1 | −0.27994 | Increases |
| activator of neutrophils | PPBP | | | | |
| Can activate [symbol] | Plasminogen Activator Inhibitor 1 - | Serum | C2D1 | −0.07995 | Increases |
| through T [symbol] - [symbol] receptor-4 | SERPINE1 | | | | |
| Innate Immunity | Peptidoglycan recognition Protein 1 - | Serum | C1D15 | 0.13385 | Increases |
| | PGLRP1 | | | | |

[symbol]

indicates data missing or illegible when filed

Patients treated with sotiga/chemo had increases in soluble proteins essential for the activation of helper T cells/B cells (CCL15) and monocytes (GSHB) (Table 24).

TABLE 24

| | | | | Median | Directional |
|---|---|---|---|---|---|
| | | | Contribution | Importance | Change from |
| Relevant Biology | Factors | Sample | Timepoint(s) | Score | Pretreatment |
| Aids in the regulation the | Amyloid Beta Precursor Protein - | Serum | C3D1 | 0.05242 | Increases |
| activation of myeloid cells | APP | | | | |
| Chemotactic factor that attracts | CCL15 | Serum | C3D1 | −0.05505 | Increases |
| T-cells and monocytes | | | | | |
| Essential to produce | Glutathione Synthetase - GHSB | Serum | C1D15 | −0.18952 | Increases |
| Glutathione | | | | | |
| T cell Function | | | | | |
| Aids in generation of | | | | | |
| proinflammatory | | | | | |
| monocytes | | | | | |
| Down-regulates TLR9- | Protein Tyrosine Phosphatase | Serum | C3D1 | 0.00444 | Increases |
| mediated activation of NF- | Receptor Type S - PTPRS | | | | |
| kappa-B, as well as production | | | | | |
| of INF, interferon alpha and | | | | | |
| interferon beta | | | | | |
| Positively regulates T and B | Transferrin Receptor TFRC | Serum | C3D1 | 0.00371 | Increases |
| cell proliferation through [symbol] | | | | | |
| uptake | | | | | |
| Involved in Innate Immunity | Cysteine Rich Secretory Protein 3 - | Serum | C3D1 | 0.65216 | Increases |
| | CRISP3 | | | | |
| reported to have both immune | Poliovirus Receptor - PVR | Serum | C1D15 | −0.09675 | Increases |
| regulatory and immune | | | | | |
| stimulatory effects | | | | | |
| Binds to TIGIT | | | | | |
| Binds to CD266 on T cells, | | | | | |
| NK, and monocytes aiding | | | | | |
| in activation | | | | | |
| Can aid in the induction of Th2 | MMP-2 | Serum | C3D1 | 0.03838 | Increases |
| polarization | | | | | |

[symbol]

indicates data missing or illegible when filed

Integrated analysis of biomarkers measured on-treatment (C2D1) was performed. In the nivo/chemo treatment arm, increased sera levels of chemokines and cytokines associated with type 1 immunity (CXCL9, CXCL10, CXCL11, and IFN-Y) were positively correlated with activated T cells (HLA-DR+, CD38+) (FIG. 15G and FIG. 15H). In the sotiga/chemo treatment arm, molecules reported to be associated with the migration of innate and adaptive immune cells increased on-treatment (GKN1 and CCL15) and positively correlated with proteins associated with DC maturation such LAMP3 and CXCL11 or activated non-naïve T cells (CD38+ or CD39+) and CCR7+B cells. (FIG. 15G and FIG. 15H).

To evaluate pharmacodynamic effects in the tumor, pretreatment and on-treatment (˜C2D1, see Methods) tumor tissue were profiled with mIF. The analysis of paired biopsies from individual patients revealed that nivo/chemo treatment led to a numerically decreased percentage of tumor cells expressing PD-L1 in all samples measured (n=5). Changes in the percentage of PD-L1 positive tumor cells were heterogeneous, decreased in one sample and increased in 2 others. The combination of sotiga/nivo/chemo resulted in a decrease in PD-L1 positive tumor cells in 5 out of 6 patient samples analyzed (FIG. 14D). For sotiga/chemo treatment, 2 of 3 patients with paired biopsies exhibited increases in tumor-infiltrating iNOS+CD80+CD68+ macrophages, an effect that was not observed for paired biopsies from patients treated with nivo/chemo or sotiga/nivo/chemo (FIG. 14E).

### Example 12. Biomarkers Associated with Survival Benefit

To identify subsets of patients who are more likely to demonstrate longer survival from a specific combination treatment, we performed exploratory analyses using comprehensive multi-omic, multi-parameter immune and tumor biomarker data. An approach of focusing on biological signals observed across multiple assays helped to identify signals of underlying biology that have maximal robustness in the context of a small Phase II study. This deep, integrated analysis approach provided a comprehensive view of tumor and immune contexture and identified numerous biomarkers that associated with survival benefit in each arm.

### Example 13. Biomarkers and Immunobiology Associated with Survival Benefit Following Nivo/Chemo

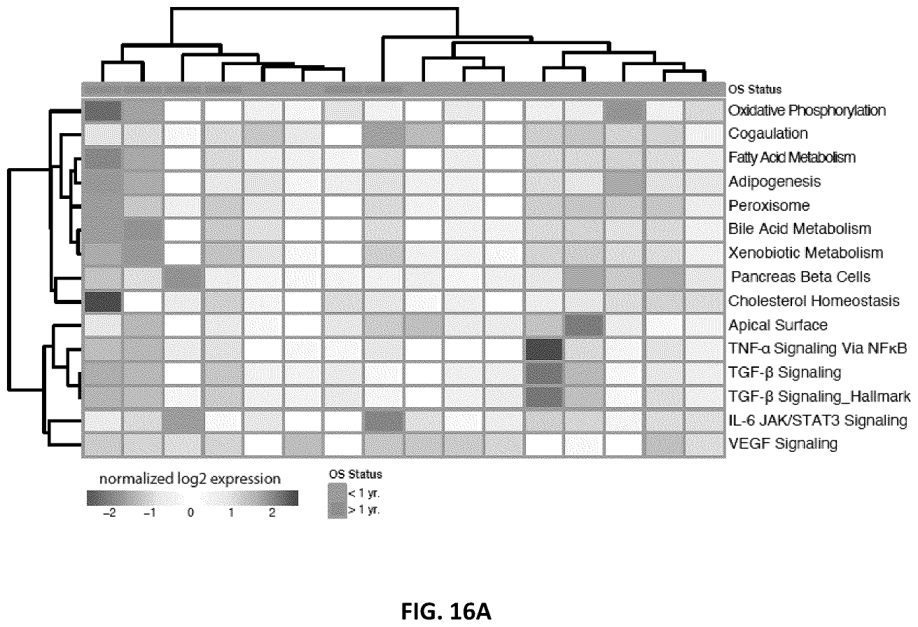

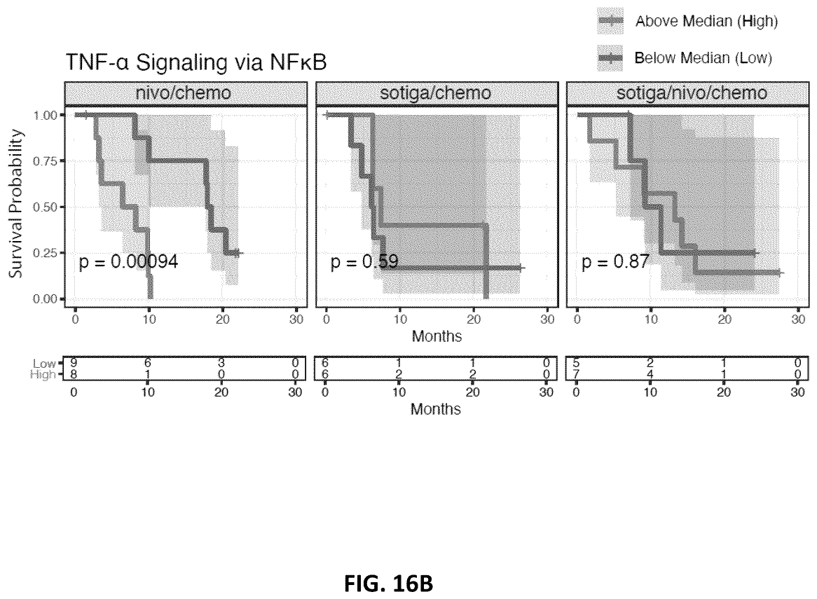

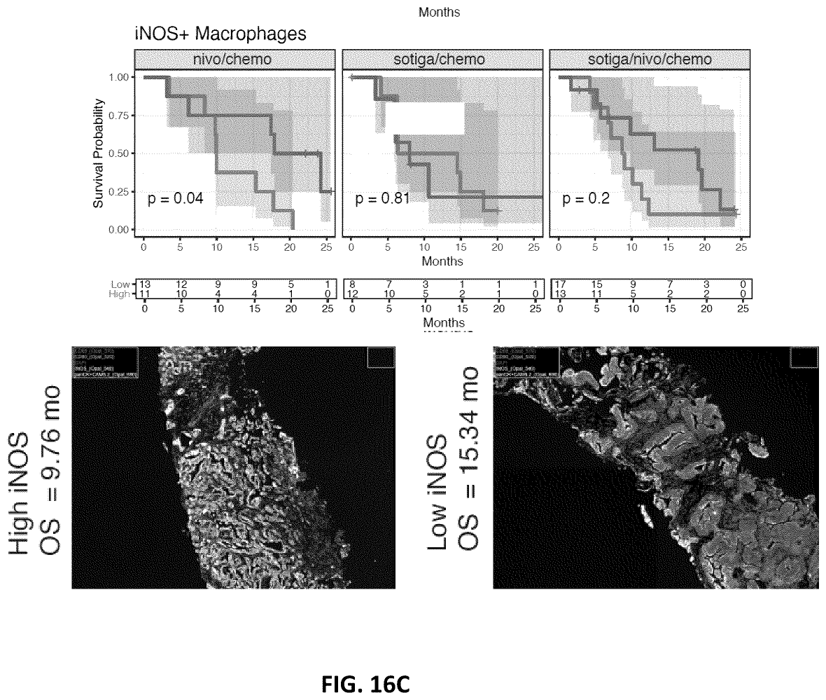

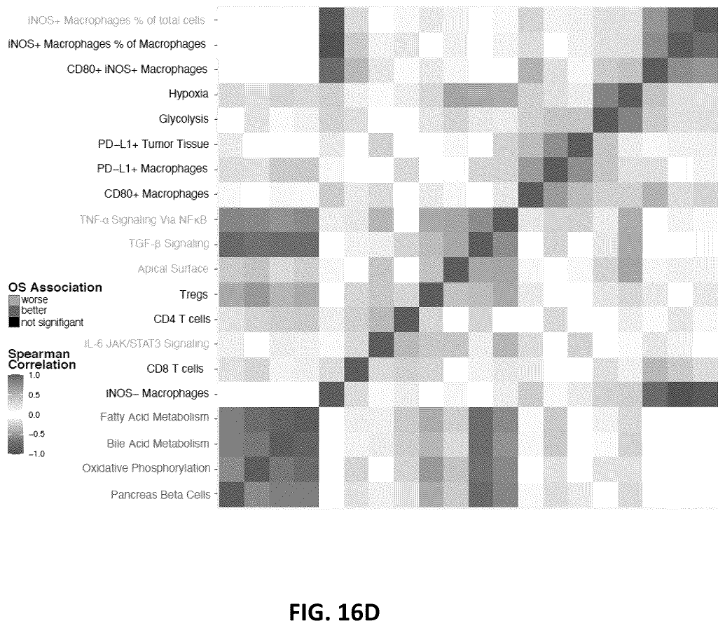

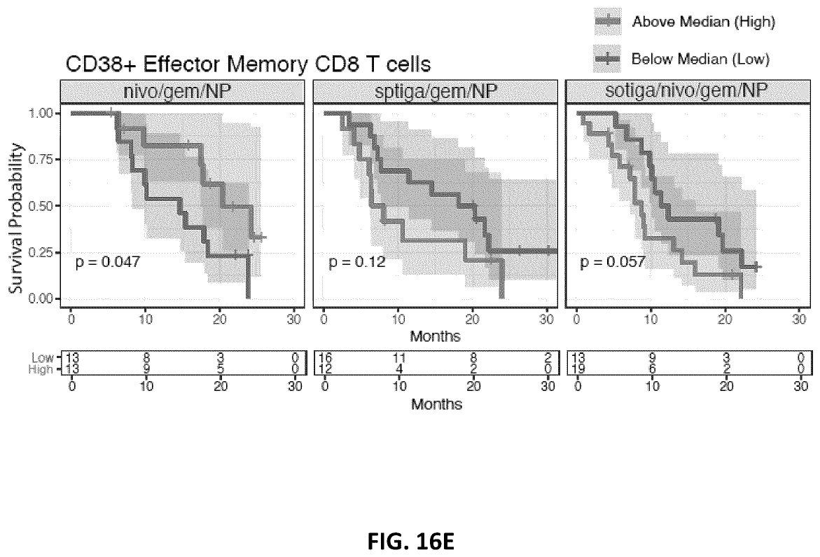

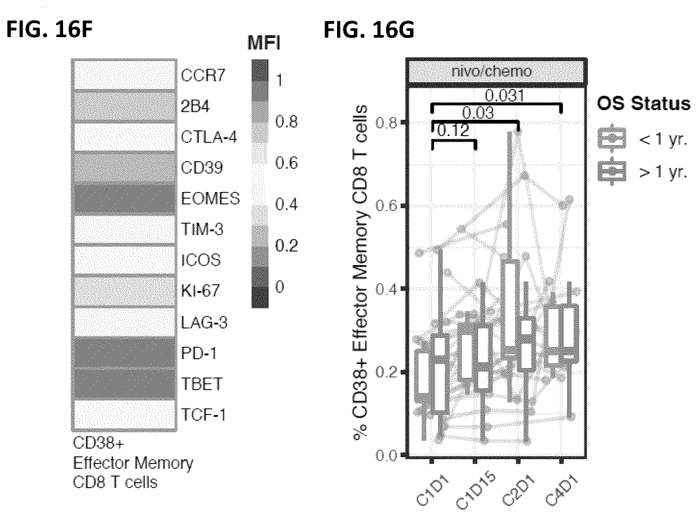

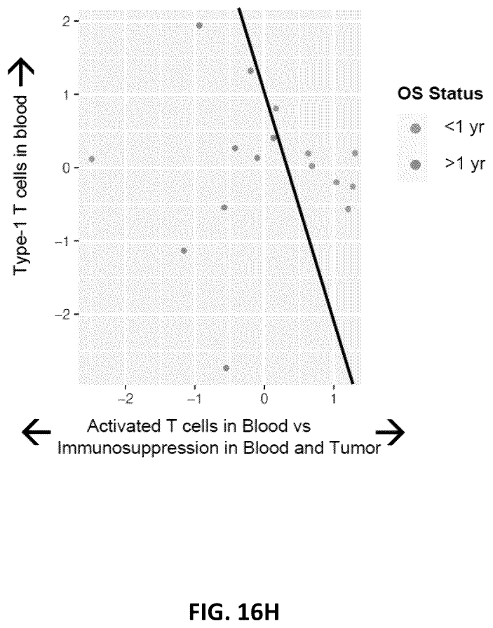

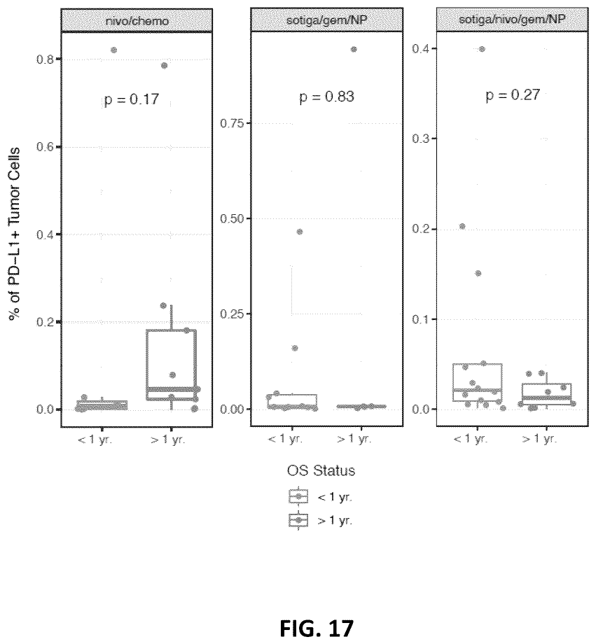

To examine the patient's tumor microenvironment (TME) prior to treatment, total RNA sequencing and mIF was analyzed. Oxidative phosphorylation, fatty acid metabolism, xenobiotic metabolism, and bile acid metabolism gene expression signatures were associated with longer survival, whereas a TGF-ß signaling signature was associated with shorter survival (FIG. 16A). Lower expression of the hallmark gene expression signatures, IL-6, TNF-α signaling via NFκB, and lower frequencies of iNOS+ macrophages by mIF, was associated with longer survival in patients treated with nivo/chemo (FIG. 16B, FIG. 16C, FIG. 16D). Higher frequencies of PD-L1+ tumor cells prior to treatment, as measured by mIF, had a weak association with greater than one year survival (FIG. 17), but did not significantly associate with longer overall survival by Kaplan-Meier analysis. Additionally, immunosuppressive factors in the circulation were associated with shorter survival (Table 25). Lower levels of nitric oxide synthase 3 and arginase-1 were associated with longer survival in patients treated with nivo/chemo (Table 25).

TABLE 25

| | | | | | Survival |
|---|---|---|---|---|---|
| | | | | | Association of |
| Relevant Biology | Biomarker Feature | Sample Type | Assay | P-value | Higher Values |
| Proliferation [symbol] | [symbol] | [symbol] | [symbol] | [symbol] | [symbol] |
| [symbol] T cells | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| Antigen [symbol] T cells | [symbol] | [symbol] | [symbol] | [symbol] | [symbol] |
| | [symbol] | [symbol] | [symbol] | [symbol] | [symbol] |
| Immunosuppressive [symbol] | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| Inflammatory [symbol] | [symbol] | [symbol] | [symbol] | [symbol] | [symbol] |
| Macrophage Inflammatory | [symbol] | [symbol] | [symbol] | [symbol] | [symbol] |
| Response | [symbol] | [symbol] | [symbol] | [symbol] | [symbol] |
| May Aid in [symbol] | [symbol] | [symbol] | [symbol] | [symbol] | [symbol] |
| Tumor [symbol] | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| Type 1 Immune Response | [symbol] | [symbol] | [symbol] | [symbol] | [symbol] |
| | [symbol] | [symbol] | [symbol] | [symbol] | [symbol] |
| [symbol] T cells | [symbol] | [symbol] | [symbol] | [symbol] | [symbol] |
| | [symbol] | [symbol] | [symbol] | [symbol] | [symbol] |
| CD4 Helper Response | [symbol] | [symbol] | [symbol] | [symbol] | [symbol] |
| DC [symbol] | [symbol] | [symbol] | [symbol] | [symbol] | [symbol] |
| Aids in [symbol] | [symbol] | [symbol] | [symbol] | [symbol] | [symbol] |

[symbol]

[symbol]

[symbol]

indicates data missing or illegible when filed

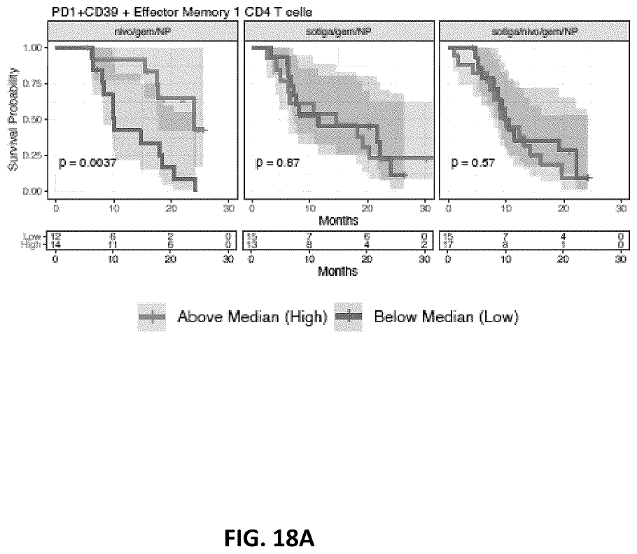

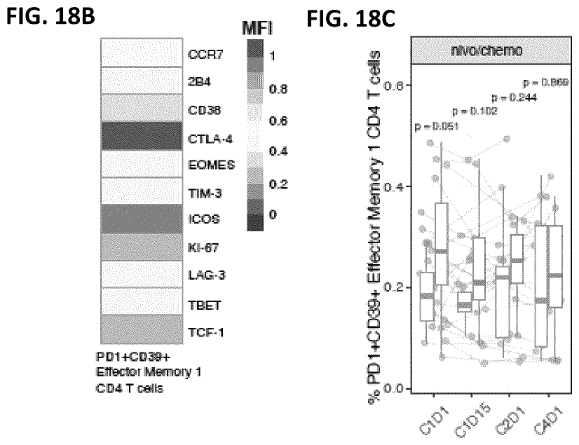

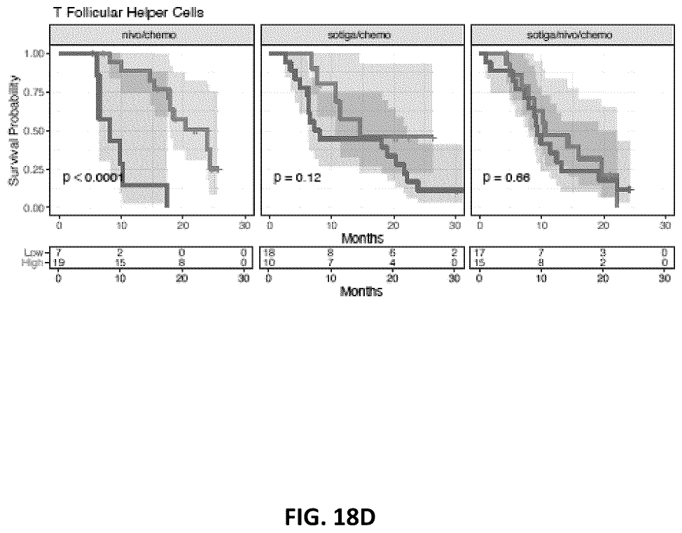

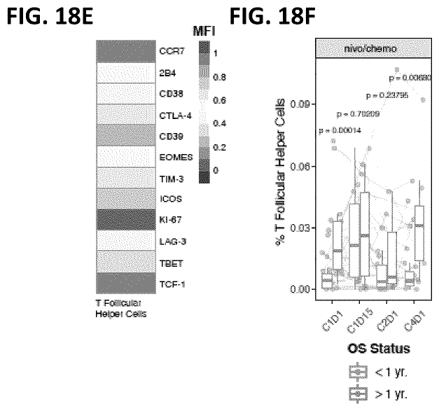

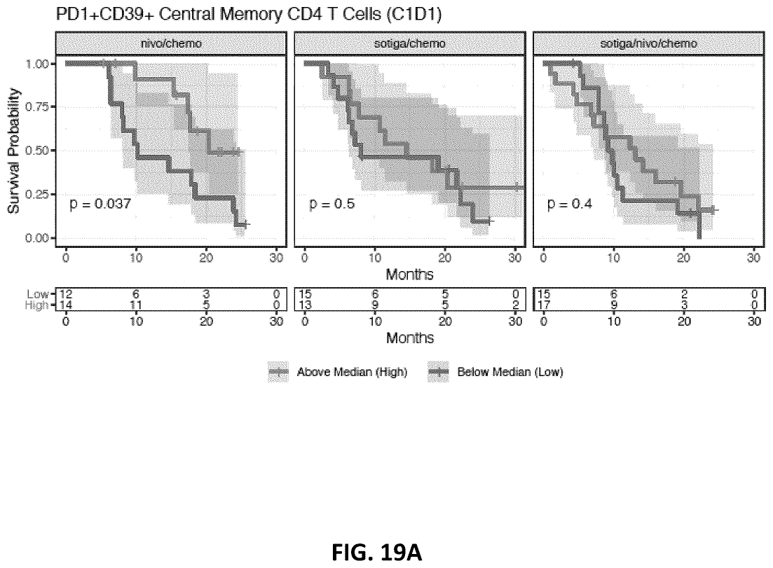

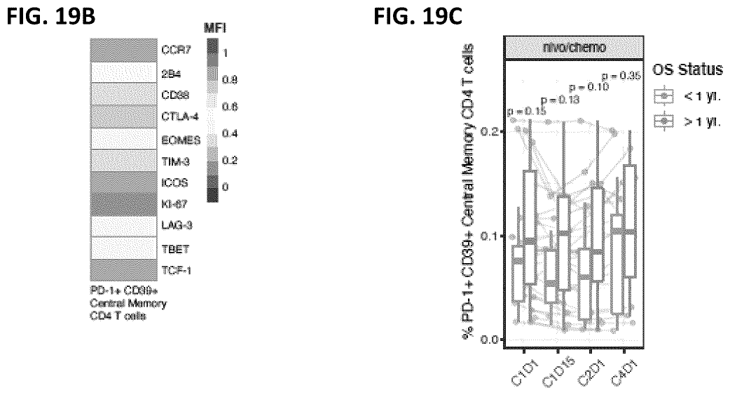

Survival benefit following nivo/chemo was associated with a diverse, immunocompetent circulating T cell response pretreatment. CD4 and CD8 T cells were classified as effector memory (EM) (FIG. 10) or central memory (CM) (FIG. 10). Effector memory T cells were further subdivided based on CCR7 expression: EM1, EM2, and EM3, (FIG. 10). Higher frequencies of activated (CD38+) EM CD8 T cells (FIG. 16E), antigen experienced (PD-1+CD39+) EM1 (FIG. 18A) and CM CD4 T cells (FIG. 19A), as well as T follicular helper cells (CD4+PD-1+CXCR5+) (FIG. 18D), were all associated with longer survival in patients treated with nivo/chemo. Activated (CD38+) EM CD8 T cells also co-expressed PD-1 and the type-1 transcription factor, Tbet (FIG. 16F). Although activated (CD38+) EM CD8 T cells increased over time, only pretreatment levels were associated with 1-year survival status (FIG. 16G). Antigen-experienced (PD-1+CD39+) EM1 and CM CD4 T cells co-expressed CTLA-4 and ICOS (FIG. 18B, and FIG. 19B). On-treatment, this cellular phenotype continued to be associated with better survival (FIG. 18C and FIG. 19C). High on-treatment abundances of T follicular helper cells, which had high expression of TCF-1 and ICOS, were associated with survival at 1 year (FIG. 18E and FIG. 18F). Multi-omic dimensionality reduction analysis of both circulating and tumor factors recapitulated these findings and revealed the primary axes of independent variance in the data, showing a separation between patients with survival >1 year and <1 year (FIG. 16H). Overall, patients with longer survival following nivo/chemo treatment had lower pretreatment levels of immunosuppressive molecules and higher pretreatment frequencies of activated, type-1 T cells, compared to patients with shorter survival (FIG. 16H).

### Example 14. Biomarkers and Immunobiology Associated with Survival Benefit Following Sotiga/Chemo

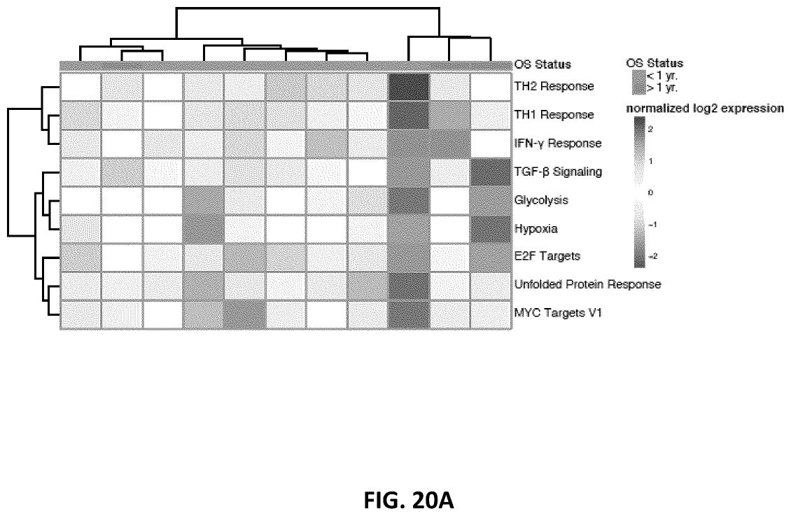

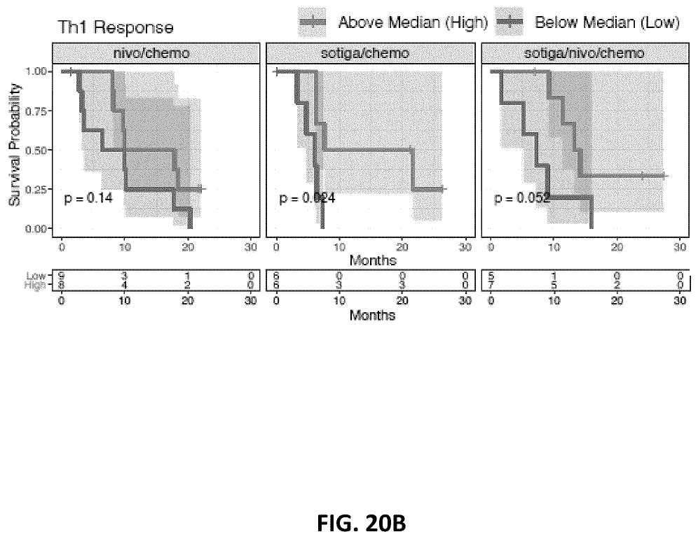

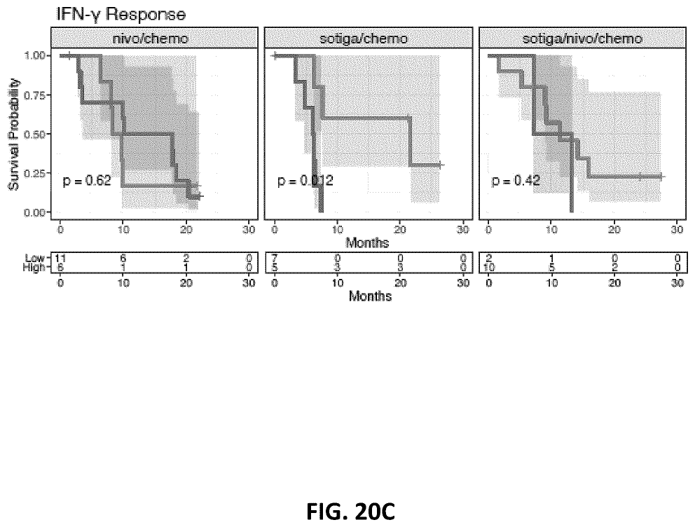

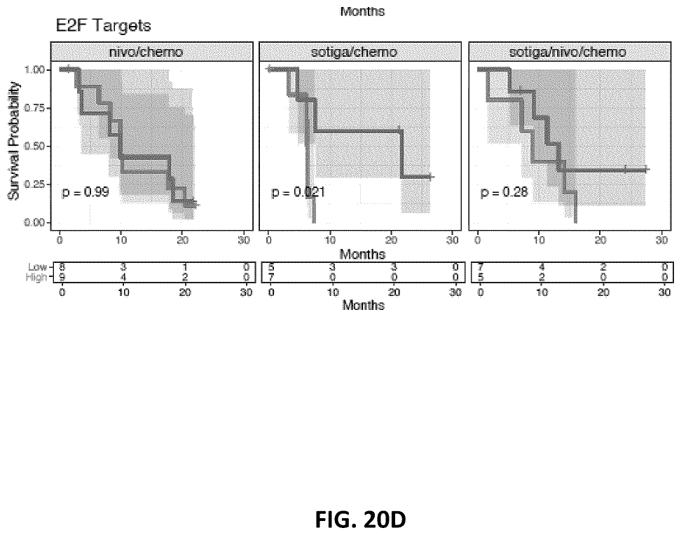

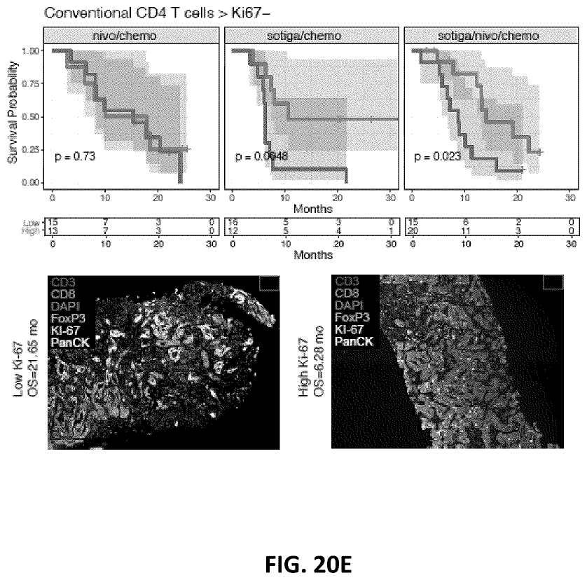

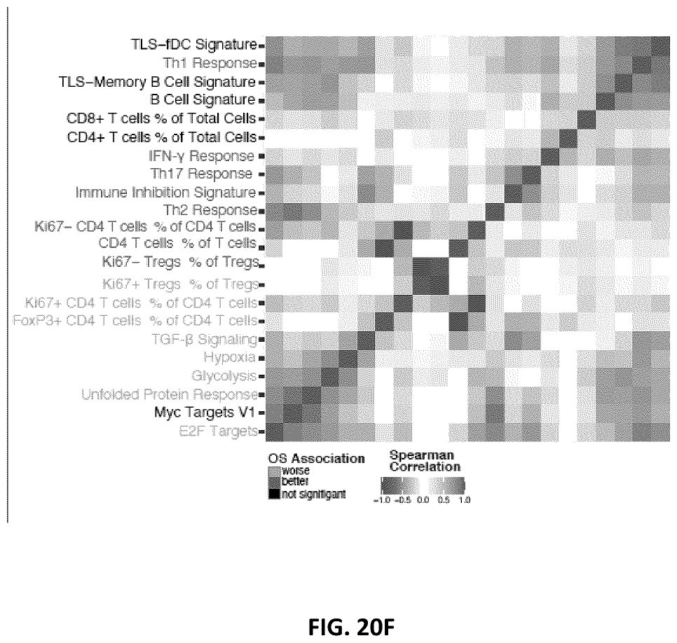

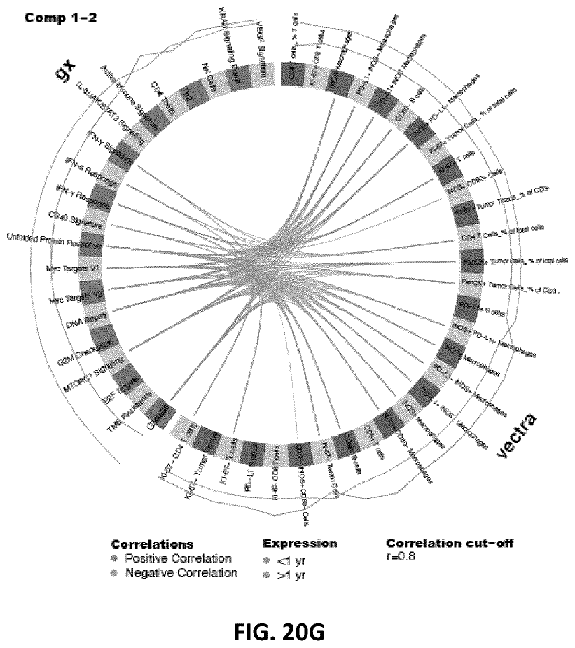

Different TME biomarkers associated with survival benefit following sotiga/chemo versus nivo/chemo treatment. Patients with longer survival after sotiga/chemo treatment had a pretreatment tumor profile with a diverse CD4 helper T cell infiltrate and lower levels of gene expression signatures and immune cell types associated with immune suppression. CD4 T cell gene expression signatures associated with longer survival included Th1 and Th2 responses, and IFN-γ signaling (FIG. 20A-20C, Table 26).

TABLE 26

| Signature | Gene List |
|---|---|
| Th1 | CXCR3, IFNG, IFNG-AS1, IL12ERB1, IL18R1, IL18RAP, STAT1, STAT4, TBX21 |
| Th2 | GATA3, IL4, IRF4, MAF, STAT5A, STAT6 |
| Th17 | AHR, BATF, CCR6, IL17A, IL17F, IL21, IL6R, RORC, RUNX1, STAT3 |
| TLS-fDC | CD2, CR1, FCER2, HLA-DRA |
| TLS-Memory B cell | CD27, CD69, CD86, CR2, CXCR3, IGHD, MS4A1 |
| B Cell | BTLA FCRL5, IDO1, IFNG, IGLL5, JCHAIN, MZB1 |
| IFN-γ Response | CD8A, CD274, LAG3, STAT1 |

Note:
This table includes gene signatures other than HALLMARK gene signatures, which can be found in the Molecular Signatures Database (MSigDB).

Patients with longer survival following sotiga/chemo treatment also had higher frequencies of tumor infiltrating non-proliferating (Ki-67−) conventional and regulatory (Foxp3+) CD4 T cells (FIG. 20E and FIG. 20F, Table 26) and lower frequencies of infiltrating proliferating (Ki-67+) CD4 T cells (FIG. 20F, Table 26) by mIF. Patients whose tumors had high E2F signaling signatures also had shorter survival (FIG. 20D, Table 26). By cross-platform analysis with DIABLO (see Methods), E2F signaling signatures positively correlated with glycolysis and hypoxia gene expression signatures and infiltrating iNOS-macrophages, which were also associated with shorter survival following sotiga/chemo treatment (FIG. 20F and FIG. 20G).

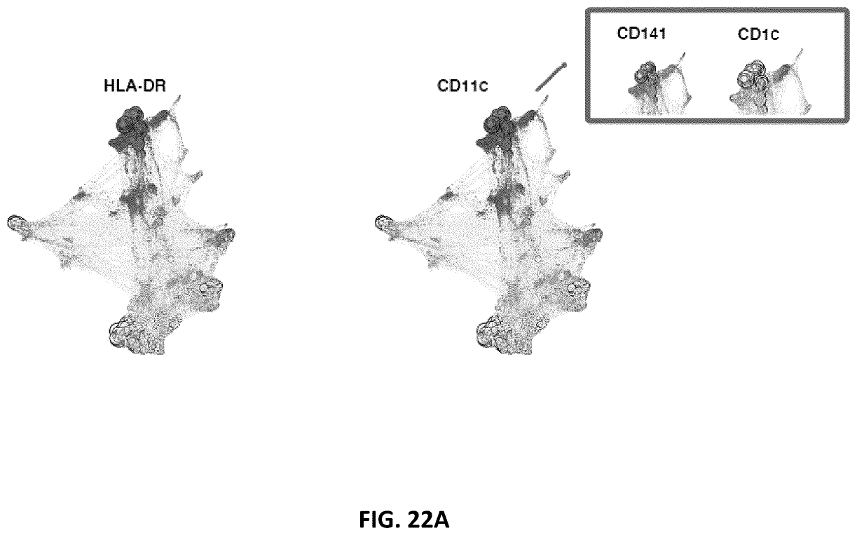

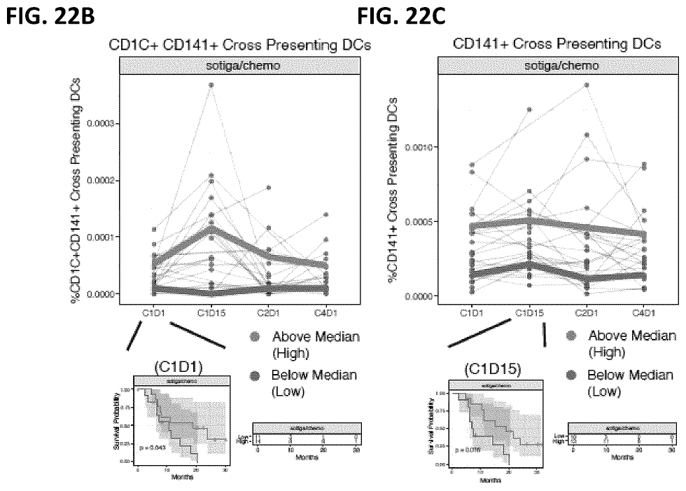

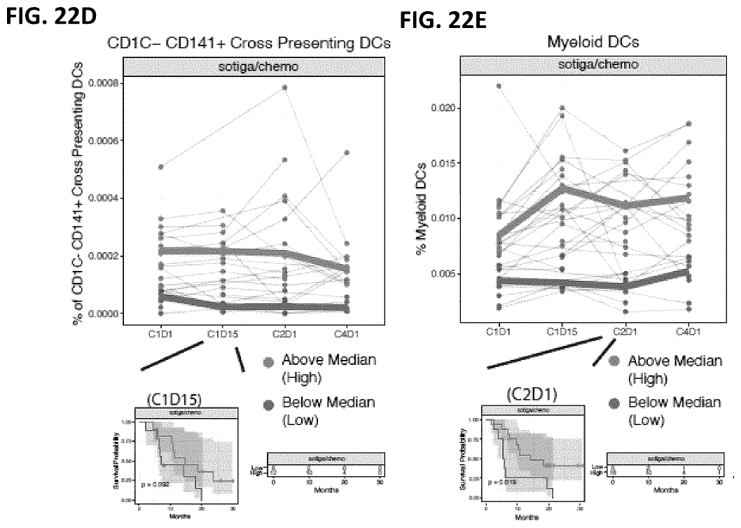

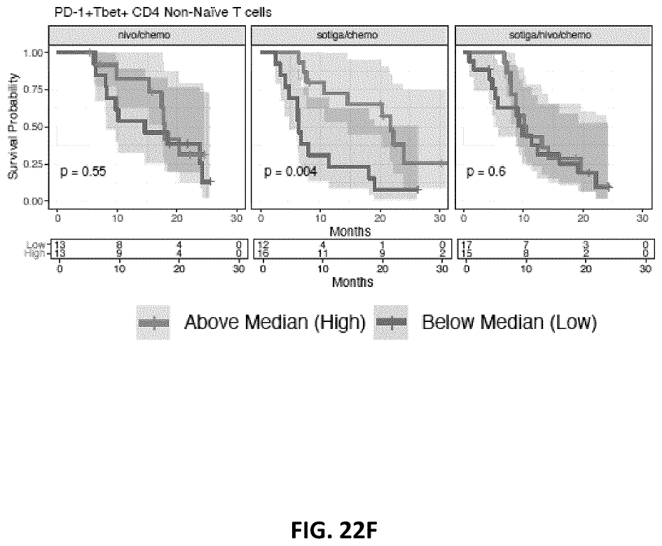

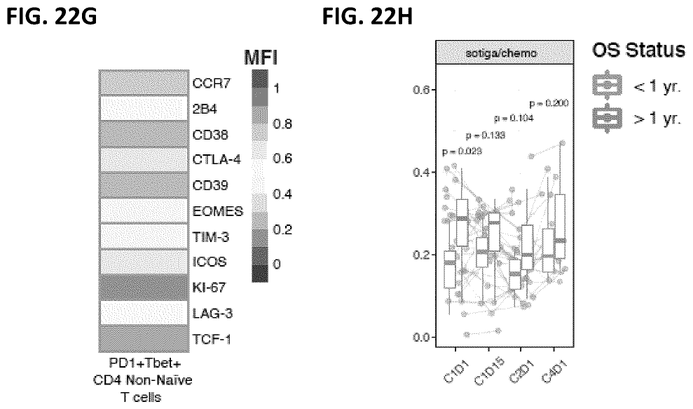

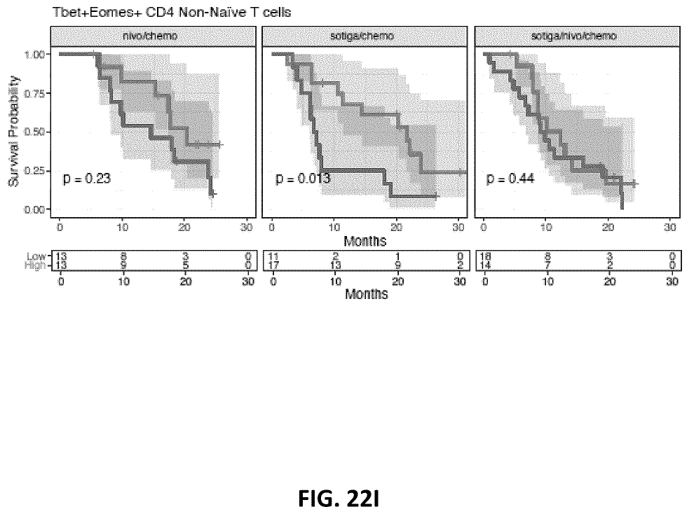

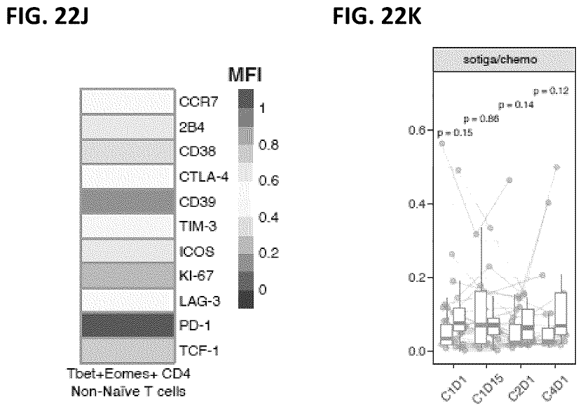

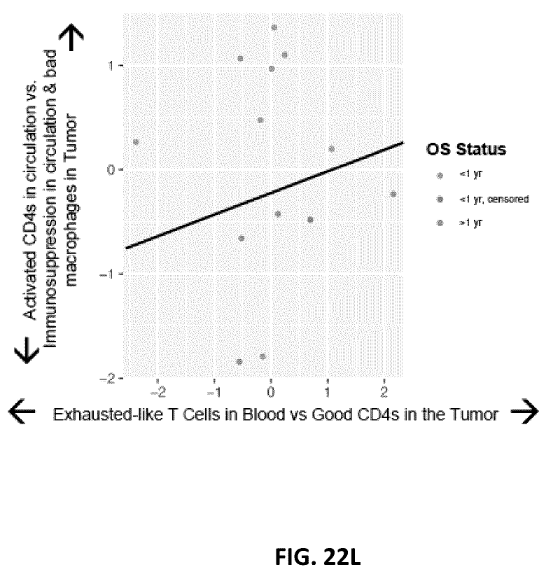

Pre-clinical data suggest that CD40 agonism results in antigen presenting cell (APC) activation, and thus we hypothesized that patients who experienced survival benefit following sotiga/chemo would have evidence of this in the circulation. We therefore performed immune profiling of pretreatment and on-treatment PBMCs using CyTOF and flow cytometry (see Methods). With unsupervised clustering analysis (FIG. 22A, see Methods), we identified multiple circulating dendritic cell (DC) subsets (FIG. 22A, Table 27) that were associated with survival prior to and following treatment with sotiga/chemo.

TABLE 27

| | | | | | Survival |
|---|---|---|---|---|---|
| | | | | | Association of |
| Relevant Biology | Biomarker Feature | Sample Type | Assay | P-value | Higher Values |
| [symbol] T regulatory cells | [symbol] | PBMCs | [symbol] | 0.023 | Longer Survival |
| Activated, possibly [symbol] | [symbol] | PBMCs | [symbol] | 0.017 | Longer Survival |
| [symbol] B cells | | | | | |
| [symbol] | [symbol] | Serum | [symbol] | 0.0003 | Shorter Survival |
| Anti-tumor Immune | IFN-γ Response | Tumor Tissue | [symbol] | 0.01 | Longer Survival |
| response | | | | | |
| Antigen Experienced T cells | [symbol] | PBMCs | [symbol] | 0.00 [symbol] | Longer Survival |
| CD4 Helper Responses | [symbol] | Tumor Tissue | [symbol] | 0.00 [symbol] | Longer Survival |
| | [symbol] | PBMCs | [symbol] | 0.0 [symbol] | Longer Survival |
| | [symbol] | Tumor Tissue | [symbol] | 0.02 | Longer Survival |
| | [symbol] | Tumor Tissue | [symbol] | 0.02 | Longer Survival |
| | [symbol] | PBMCs | [symbol] | 0.000 [symbol] | Longer Survival |
| Checkpoint Molecules | [symbol] | PBMCs | [symbol] | 0.0 [symbol] | Shorter Survival |
| | [symbol] | PBMCs | [symbol] | 0.023 | Longer Survival |
| | [symbol] | PBMCs | [symbol] | 0.001 | Longer Survival |
| Chemotactic for Resting T | [symbol] | Serum | [symbol] | 0.00 [symbol] | Shorter Survival |
| cells | | | | | |
| Co- [symbol] | [symbol] | PBMCs | [symbol] | 0.00 [symbol] | Longer Survival |
| | [symbol] | PBMCs | [symbol] | 0.0 [symbol] | Longer Survival |
| Dendritic Cell Biology | [symbol] | PBMCs | [symbol] | 0.042 | Longer Survival |
| Antigen Processing & | [symbol] | Serum | [symbol] | 0.0z,899; | Longer Survival |
| Presentation | [symbol] | Serum | [symbol] | 0.0 [symbol] | Longer Survival |
| | [symbol] | Serum | [symbol] | 0.0 [symbol] | Longer Survival |
| Immunosuppressive | [symbol] | Serum | [symbol] | 0.0 [symbol] | Shorter Survival |
| | [symbol] | Serum | [symbol] | 0.00 [symbol] | Shorter Survival |
| | [symbol] | Serum | [symbol] | 0.00 [symbol] | Shorter Survival |
| | [symbol] | Serum | [symbol] | 0.01 [symbol] | Shorter Survival |
| | [symbol] | Serum | [symbol] | 0.029 | Shorter Survival |
| | [symbol] | Tumor Tissue | [symbol] | 0.02 [symbol] | Longer Survival |
| | [symbol] | Tumor Tissue | [symbol] | 0.02 | Shorter Survival |
| | [symbol] | Tumor Tissue | [symbol] | 0.0 [symbol] | Shorter Survival |
| | [symbol] | Tumor Tissue | [symbol] | 0.0 [symbol] | Shorter Survival |
| | [symbol] | Tumor Tissue | [symbol] | 0.0 [symbol] | Shorter Survival |
| | [symbol] | PBMCs | [symbol] | 0. [symbol] | Shorter Survival |
| | [symbol] | Serum | [symbol] | 0.00 [symbol] | Shorter Survival |
| Induction of T cell | [symbol] | [symbol] | [symbol] | 0.0 [symbol] | Shorter Survival |
| [symbol] | | | | | |
| Innate Immunity | [symbol] | Tumor Tissue | [symbol] | 0.00 [symbol] | Longer Survival |
| | [symbol] | Tumor Tissue | [symbol] | 0.03 [symbol] | Longer Survival |
| Pro-inflammatory | [symbol] | PBMCs | [symbol] | 0.0 [symbol] | Longer Survival |
| | [symbol] | PBMCs | [symbol] | 0.002 | Longer Survival |
| | IL-6 | Serum | [symbol] | 0.00 [symbol] | Shorter Survival |
| Type 1 Immunity | [symbol] | PBMCs | [symbol] | 0.002 | Longer Survival |
| | [symbol] | PBMCs | [symbol] | 0.0 [symbol] | Longer Survival |
| Type 2 Immunity | IL-4 | PBMCs | [symbol] | 0.0 [symbol] | Longer Survival |
| Tumor Metabolism, | [symbol] | Tumor Tissue | [symbol] | 0.0 [symbol] | Shorter Survival |
| Glycolytic TME | | | | | |
| Requires Further | [symbol] | PBMCs | [symbol] | 0.0 [symbol] | Longer Survival |
| Investigation | | | | | |

Biomarker associated with shorter survival
Biomarker associated with longer survival [symbol]
indicates data missing or illegible when filed

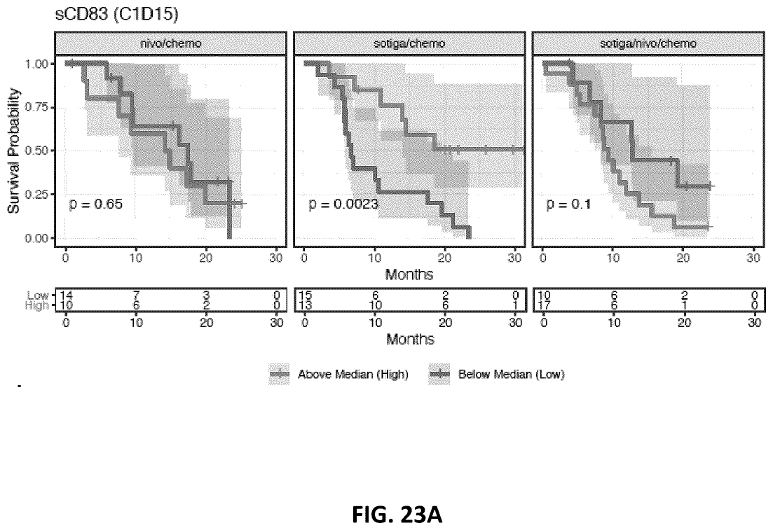

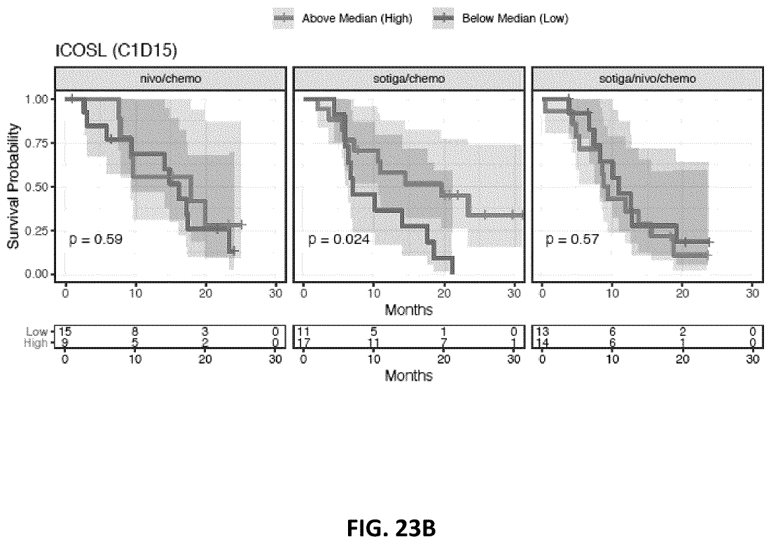

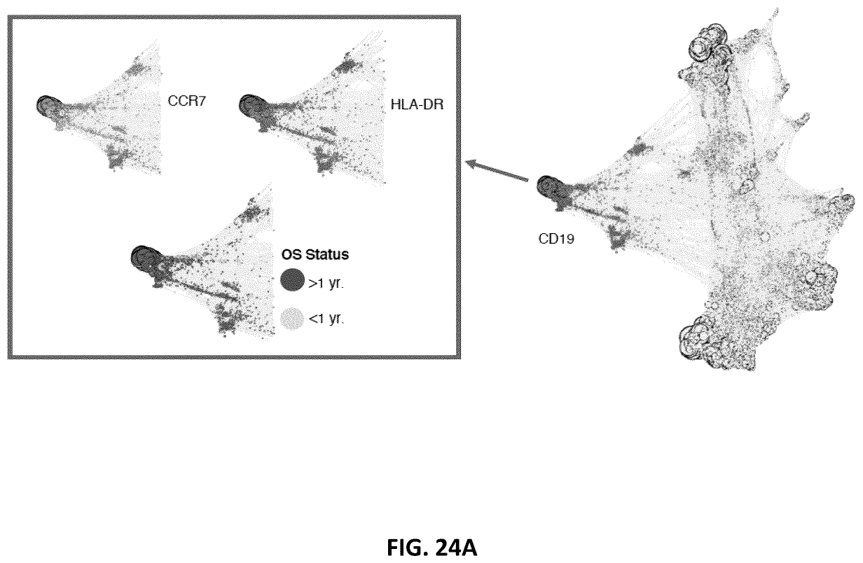

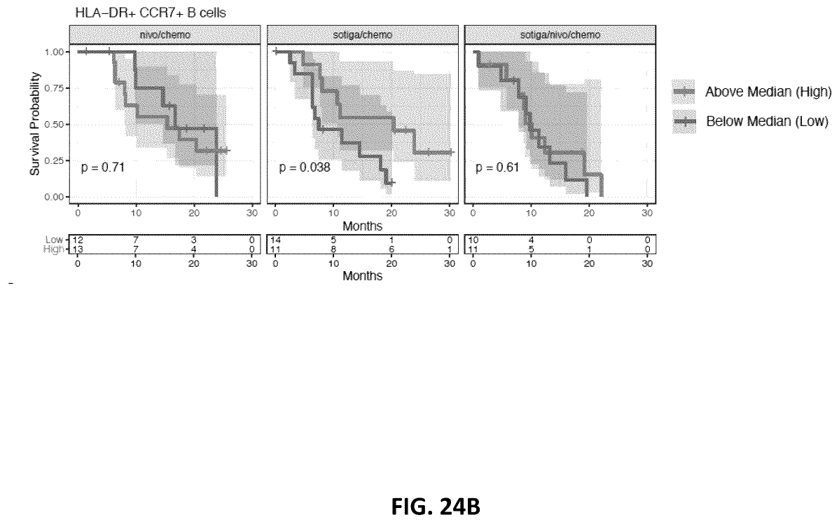

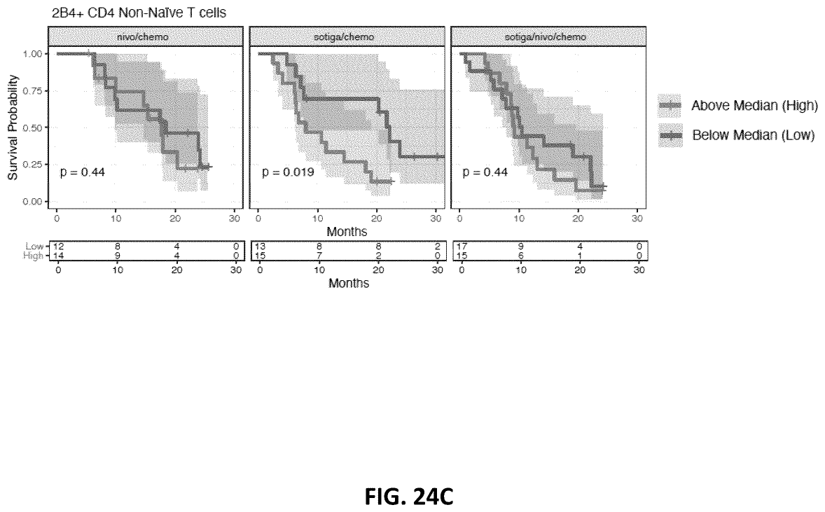

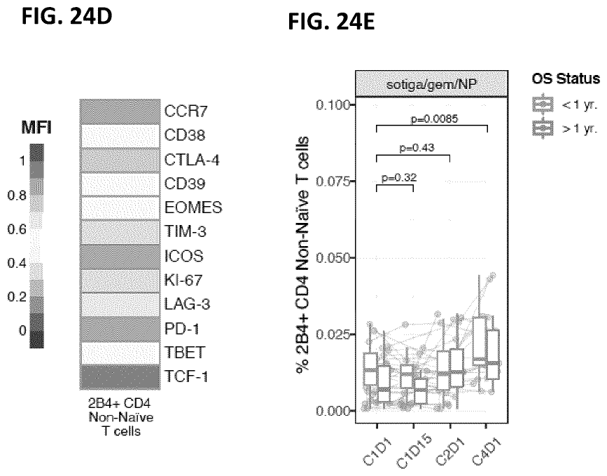

Patients with longer overall survival had higher frequencies of CD1c+CD141+DCs (FIG. 22B, Table 27) prior to treatment. On-treatment, higher frequencies of CD141+DCs, with reduced CD1c co-expression, were associated with longer survival (C1D15, FIG. 22C and FIG. 22D). Higher frequencies of conventional DCs (cDC; FIG. 9, and Table 19) on-treatment (C2D1) were also associated with longer survival (FIG. 22E FIG. 5e, Table 27). Consistent with maturation of DCs, higher concentrations of soluble CD83 and soluble ICOSL on-treatment were also associated with longer survival in patients treated with sotiga/chemo (FIG. 23). Patients with longer survival following sotiga/chemo also had higher frequencies of circulating HLA− DR+ CCR7+ B cells prior to treatment (FIG. 24A, FIG. 24B, Table 27). Overall, patients with longer survival after sotiga/chemo treatment, in contrast to those with longer survival after nivo/chemo treatment, had higher frequencies of circulating DCs and B cell frequencies in circulation prior to treatment, with phenotypic changes in the APC compartment.

In addition to an activated APC compartment, we also found that pretreatment frequencies of key CD4 T cell populations were associated with survival benefit following sotiga/chemo treatment. Higher pretreatment frequencies of circulating Type-1 helper (Tbet+Eomes+) and antigen-experienced (PD1+Tbet+) non-naïve CD4 T cells were associated with longer survival in patients treated with sotiga/chemo (FIG. 22F and FIG. 22G, Table 27). PD-1+Tbet+ non-naïve CD4 T cells expressed high levels of TCF-1, whereas the Tbet+Eomes+ non-naïve CD4 T cells expressed high levels PD-1 (FIG. 22G and FIG. 22J). Higher frequencies of both populations of non-naïve CD4 T cells prior to treatment were associated with survival benefit and frequencies of this phenotype stayed relatively consistent on-treatment (FIG. 22H and FIG. 22K). Lower frequencies of circulating non-naïve CD4 T cells expressing 2B4 prior to treatment were also associated with longer survival (FIG. 24C, Table 26). 2B4 expression on CD8 T cells has been associated with an exhausted phenotype, and these cells co-expressed other molecules associated with an exhausted or anti-inflammatory phenotype; PD-1, CTLA-4, LAG-3 and did not express Ki-67 (FIG. 24D). Additionally, the frequency of this phenotype increased on-treatment (C4D1) (FIG. 24E). Overall, type-1 (Tbet+) CD4 T cells in circulation prior to treatment associated with survival benefit following sotiga/chemo, whereas higher levels of potentially dysfunctional 2B4+CD4 T cells were associated with shorter survival.

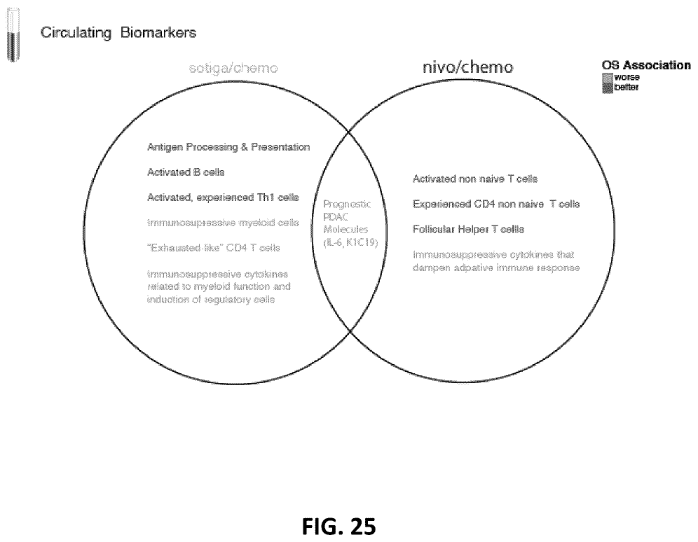

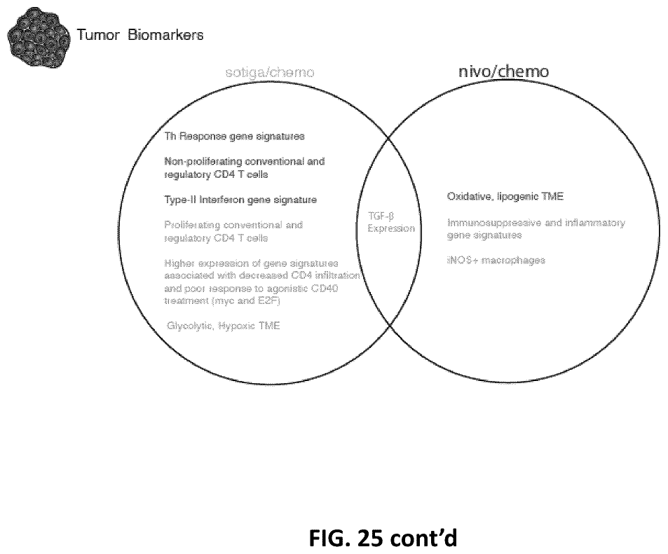

Thus, pretreatment biomarker profiles in both tissue and blood that associated with survival benefit after sotiga/chemo and nivo/chemo treatment were distinct (FIG. 25). As all patients received the same chemotherapy backbone, these predictive markers are suggested to not merely associate with prognosis or chemotherapy treatment. This conclusion is strengthened by the strong mechanistic relationship of each set of biomarkers to the CD40 and PD-1 axis.

### Example 15. Biomarkers and Immunobiology Associated with Survival Benefit Following Sotiga/Nivo/Chemo

In this study, the sotiga/nivo/chemo treatment resulted in no survival benefit over the historical control from chemo alone (FIG. 21A). In multi-omic biomarker analysis, we found that biomarkers that associated with longer survival following sotiga/chemo and nivo/chemo individually were not predictive for sotiga/nivo/chemo treatment (Table 28).

TABLE 28

| | | | | | Survival |
|---|---|---|---|---|---|
| | | | | | Association of |
| Relevant Biology | Biomarker Feature | Sample Type | Assay | P-value | Higher Values |
| “Late Activated/Exhausted” | [symbol] | PBMCs | [symbol] | 0.048 | Longer Survival |
| T cells | [symbol] | PBMCs | [symbol] | 0.017 | Shorter Survival |
| Activated T cells | IL-2 | Serum | [symbol] | 0.009 | Shorter Survival |
| | [symbol] | PBMCs | [symbol] | 0.010 | Shorter Survival |
| | [symbol] | PBMCs | [symbol] | 0.016 | Shorter Survival |
| | [symbol] | PBMCs | [symbol] | 0.013 | Longer Survival |
| Activated Type 1 Immune | Th1 response | Tumor Tissue | [symbol] | 0.0 [symbol] | Longer Survival |
| Response | | | | | |
| Co- [symbol] | [symbol] | Serum | [symbol] | 0.000 [symbol] | Shorter Survival |
| Immunosuppressive | [symbol] | Serum | [symbol] | 0.0000 [symbol] | Shorter Survival |
| | [symbol] | Serum | [symbol] | 0.000 [symbol] | Shorter Survival |
| | [symbol] | Serum | [symbol] | 0.001 | Shorter Survival |
| | [symbol] | Serum | [symbol] | 0.018 | Shorter Survival |
| | [symbol] | Serum | [symbol] | 0.000 [symbol] | Shorter Survival |
| Th2 Helper Response | IL-1 [symbol] | Serum | [symbol] | 0.00 [symbol] | Shorter Survival |
| | IL-4 | Serum | [symbol] | 0.011 | Shorter Survival |
| Proliferating, Activated T | [symbol] | PBMCs | [symbol] | 0.013 | Shorter Survival |
| regulatory cells | | | | | |

Biomarker associated with shorter survival
Biomarker associated with longer survival [symbol]
indicates data missing or illegible when filed

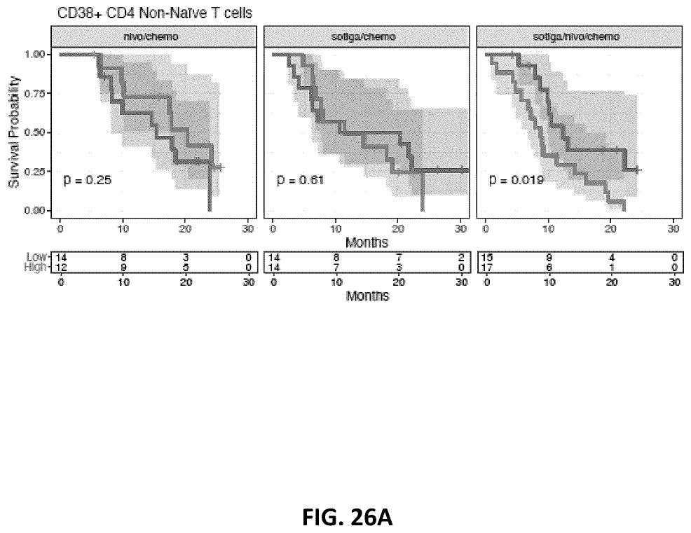

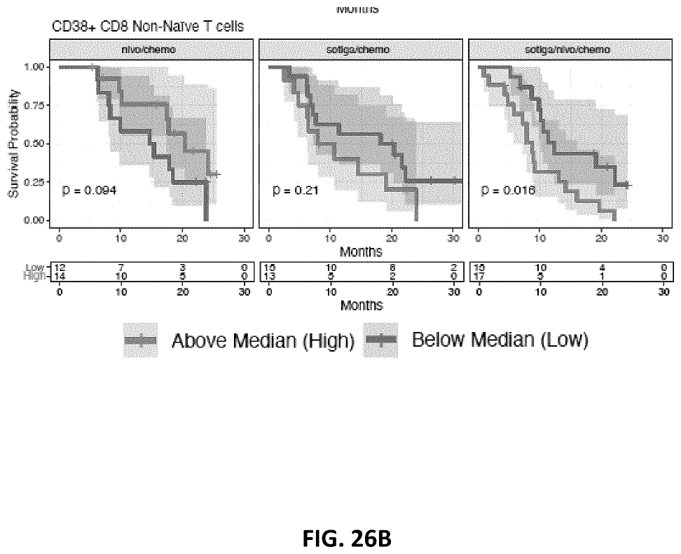

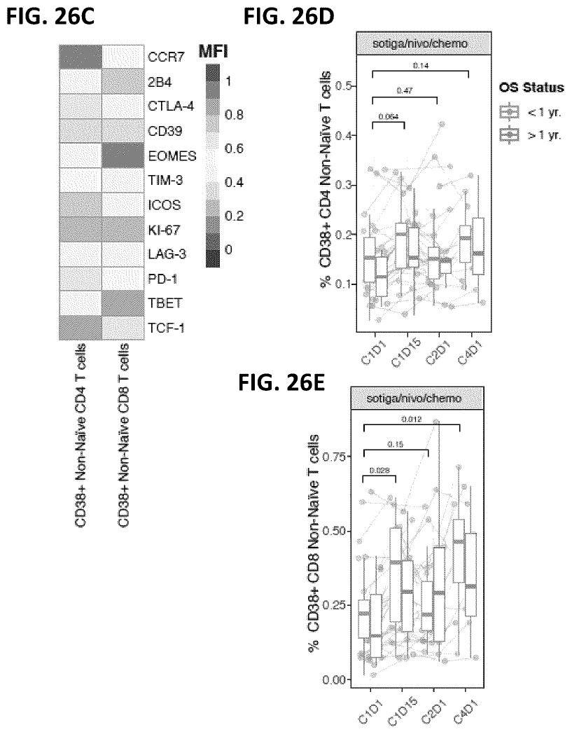

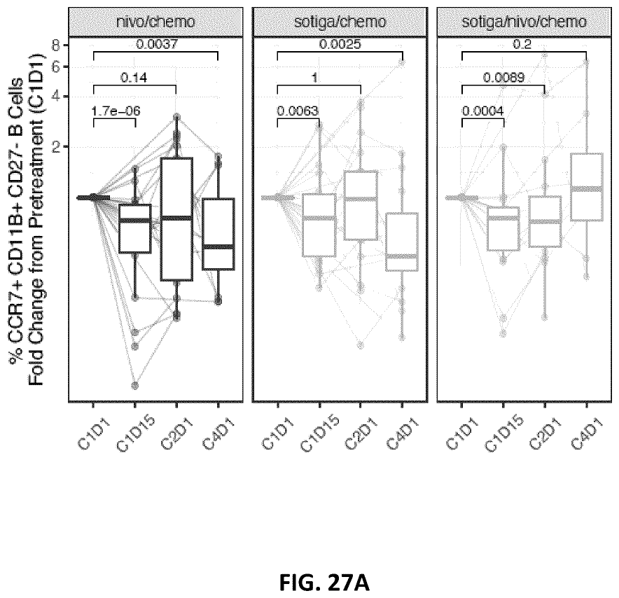

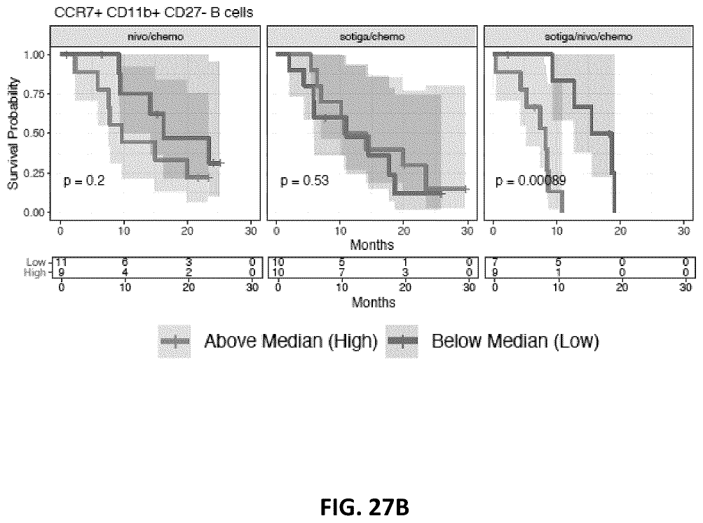

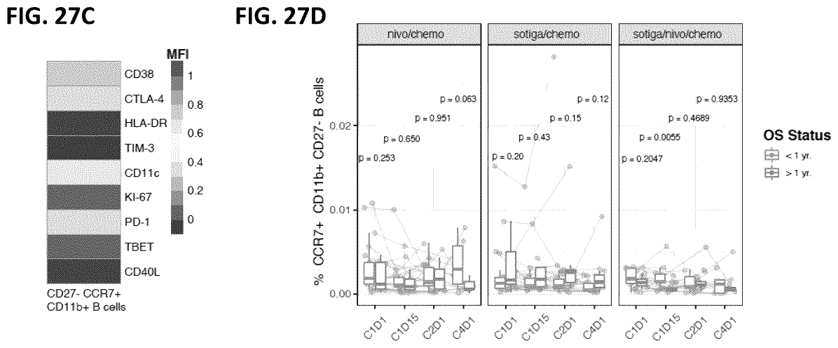

However, we identified several unique cell populations that were associated with longer survival following sotiga/nivo/chemo treatment, including lower frequencies of activated CD38+ non-naïve CD4 (FIG. 26A, Table 28) and CD8 (FIG. 26B, Table 28) T cells. The CD38+ non-naïve CD4 T cell population also expressed high levels of TCF-1 and activation markers including CTLA-4, PD-1, ICOS, whereas the CD38+ non-naïve CD8 T cell population expressed high levels of PD-1, Tbet, Eomes, TCF-1 and 2B4 (FIG. 26C). The frequency of this cellular phenotype increased on-treatment but did not continue to associate with shorter survival (FIG. 26D-26E). In the nivo/chemo treatment arm, higher frequencies of similar activated T cell populations associated with longer survival, but there was no such association observed in the sotiga/chemo treatment arm (FIG. 16F, FIG. 16G, FIG. 26A, FIG. 26B, Table 25). In addition to the CD38+ T cell populations, lower frequencies of CCR7+CD11b+CD27− B cells on-treatment (C1D15) were associated with longer survival (FIG. 27B) following sotiga/nivo/chemo treatment. Importantly, the frequency of this phenotype increased on-treatment in the sotiga/nivo/chemo arm but decreased in the other arms (FIG. 27A). These cells co-expressed CD40, HLA-DR, CD11c, and CD38 and were associated with worse survival on-treatment (C1D15) (FIG. 27C and FIG. 27D). All together, these data suggest that higher frequencies of chronically activated T cells prior to and on-treatment, and the presence of CCR7+ CD11b+ CD27− B cells on-treatment, might relate to shorter survival following treatment with sotiga/nivo/chemo.

### DISCUSSION

In this multi-center, randomized Phase II clinical study, known as PRINCE, in patients with mPDAC, efficacy and mechanisms of sotiga±nivo±chemo were evaluated in the first-line therapeutic setting. The Phase Ib portion of this study demonstrated that sotiga/chemo±nivo is tolerable, clinically active, and a potentially novel chemoimmunotherapy combination for this disease¹³. The randomized design resulted in relatively balanced demographics and baseline disease characteristics between the 3 treatment arms. Although not powered to compare between arms, enrolling 3 treatment arms concurrently allowed assessment of the relative contribution of sotiga and nivo in combination with chemo.

The nivo/chemo arm met the primary endpoint of an increase in the I-year OS rate (57.3%, p=0.007) against a historical control of 35% for the gemcitabine/nab-paclitaxel chemo regimen. The sotiga/chemo arm approached significance (48.1%, p=0.062) but the sotiga/nivo/chemo arm did not demonstrate an overall survival improvement (41.3%, p=0.223). ORR was highest for nivo/chemo (50%); however, many of the responses observed in this arm had short duration and were not confirmed by a subsequent scan.

All combination treatments were well-tolerated and the safety profile was similar to that observed in previous studies^{12,13}. Specifically, no additive toxicities were observed in the sotiga/nivo/chemo arm. Standard chemo treatment guidelines were followed in this study, allowing for continuous treatment until progression. Some accumulation of toxicity may be attributed to this long-term exposure to chemo; thus, future studies should consider response-based chemo holidays.

One limitation of this study is the lack of a chemo control arm, which hampers our ability to assess the survival benefit against contemporaneous control patients. Second, this study enrolled patients across a small number of tertiary care cancer centers. Although we benchmarked overall survival against the initial, definitive study of gemcitabine/nab-paclitaxel, subsequent phase III studies have reported higher 1-year overall survival rates of approximately 40-45%^{15,16}. However, these rates are numerically less than the 1-year overall survival rates observed for nivo/chemo and sotiga/chemo.

We performed multi-omic, multi-parametric biomarker analyses to better understand immune pharmacodynamic effects as well as the mechanisms of response and resistance with the chemoimmunotherapy combinations. Overall, findings of circulating cells, proteins and tumor tissue biomarkers aligned with the expected mechanism of action of either PD-1 blockade and/or CD40 activation. Although we observed some pharmacodynamic patterns evident across all cohorts, likely related to the identical chemotherapeutic regimen used in this study, many patterns were specific to a particular cohort and therefore not chemotherapy-specific. We observed increases in proliferating non-naïve CD4 and CD8 T cells on-treatment in all three treatment arms, consistent with previously published reports on the mechanisms of action for nivo and sotiga in other diseases^{17,18}. This increase in proliferating T cells was of greater magnitude in the two arms containing nivo, as expected¹⁸. However, in T cell populations, as well as circulating proteins and tumor tissue samples, unique immune pharmacodynamic effects for nivo/chemo and sotiga/chemo were individually identified. These data indicate that the immune therapies evaluated here have distinct activity over and above the chemotherapeutic impact in a subset of patients. Many of these pharmacodynamic effects were somewhat attenuated in the sotiga/nivo/chemo arm, potentially indicative of a decreased or antagonistic effect when all dual immunotherapy/chemo are used in combination.

In addition to pharmacodynamic effects, we examined biomarkers associated with survival. The multi-omic, multi-parameter, exploratory translational analysis in this study revealed that both nivo/chemo and sotiga/chemo demonstrated benefit in a subset of patients that can be identified by various tumor and circulating predictive biomarkers. Further, these analyses revealed that tumor and circulating response signatures identified for sotiga/chemo were unique from the ones identified for nivo/chemo, reflecting the distinct mechanisms of response to each immune intervention. It is important to note that this retrospective analysis is meant to be hypothesis-generating for future studies, and a prospective study is needed to demonstrate that these biomarkers are truly predictive of survival following these regimens.

We identified factors associated with improved survival following nivo/chemo treatment from pretreatment tumor samples, including lower expression of immunosuppression and inflammatory signatures, as well as lower frequencies of inflammatory macrophages. Lower frequencies of circulating cytokines associated with suppressive adaptive immune function were associated with longer overall survival. Survival following nivo/chemo was also associated with a diverse circulating T cell compartment, comprising of antigen-experienced and activated type-1 skewing (Tbet+) CD4 and CD8 T cells, and higher frequencies of circulating TFH cells, which may represent cells that modulate the TME^{19,20}. Many of these biomarkers could potentially be used as a pretreatment patient selection criterion for future studies, although feasibility of translating the biomarker into an assay for patient selection, especially as it relates to the timeframe from biopsy to biomarker analysis to permit clinical decision making. For example, circulating activated, antigen-experienced (PD-1+CD39+) T cell populations may make an attractive biomarker, as these populations are abundant in blood and easily and quickly measured.

In the sotiga/chemo arm, survival benefit was associated with a broad helper T cell infiltration in the tumor microenvironment. We identified an association between higher frequencies of both non-proliferative conventional and regulatory CD4+ T cells in the tumor with longer survival. In addition, consistent with prior reports²¹, gene signatures of a glycolytic or hypoxic TME prior to treatment was associated with shorter survival. This metabolic phenotype positively correlated with high tumoral expression of E2F and MYC signaling. Increased E2F and myc signaling within the TME have been reported to hinder both CD4+ T cell infiltration and response to agonistic CD40 antibodies in preclinical models²². In circulation, higher frequencies of antigen experienced, type-1 skewing CD4+ T cells prior to treatment are associated with survival to sotiga/chemo. Additionally, higher circulating frequencies of HLA− DR+ CCR7+ B cells (which may indicate the presence of geminal centers²³) prior to treatment were associated with longer survival to sotiga/chemo treatment. Fittingly with the agonistic CD40 mechanism of action, multiple DC subsets were also strongly associated with longer survival to sotiga/chemo treatment. Prior to treatment, circulating levels of CD1c+CD141+cross presenting dendritic cells were associated with longer survival. However, on-treatment CD1c+DCs were not associated with survival. Instead, higher frequencies of CD1c− CD141+cross-presenting DCs were associated with survival. The loss of CD1c expression has been reported to be associated with stronger cross presentation, and previous studies have suggested that agonistic CD40 treatment induces cross presenting DCs and may promote epitope spreading²⁴⁻²⁶. Additionally, several immunosuppressive signatures associated with poorer outcomes to sotiga/chemo treatments. These include higher frequencies of m-MDSCs, “exhausted-like” CD4 T cells, and chemokines/cytokines associated with suppressive function that were associated with shorter survival, suggesting these immune features may subvert successful response to sotiga/chemo. From a practical standpoint, baseline assessment of CD4 T cells may provide the most tractable biomarker for patient selection for sotiga/chemo in subsequent studies.

For sotiga/nivo/chemo, relatively few tumor and circulating immune biomarkers were associated with survival and, in particular, biomarkers associated with longer survival in the sotiga/chemo and nivo/chemo monotherapy immunotherapy treatment arms were not relevant following sotiga/nivo/chemo. We hypothesize that this arm resulted in systemic hyperactivation of the immune system, leading to a less functional immune state and thus decreased anti-tumor immunity. Indeed, a specific population of CD38+CD4 and CD8 T cells was associated with shorter survival in response to the sotiga/nivo/chemo combination treatment. The immunologic phenotype of these cells suggests that excessive T cell activation leads to a terminally exhausted state²⁷ unique to this chemoimmunotherapy combination. Additionally, on-treatment (C4D1), the dual immunotherapy combination of sotiga/nivo/chemo treatment had increases in circulating CCR7+CD11b+ CD27− B cells that tracked with shorter survival at both this timepoint and earlier on-treatment (C1D15). The expression of CD11b on B cells has been associated with a tolerogenic or regulatory response in the lupus setting²⁸, and is postulated to have a dampening effect on antitumor immunity. Furthermore, these findings align with recent preclinical work in glioma that suggest agonistic CD40 impairs response to PD-1 blockade in part through the induction of regulatory B cells²⁹. Thus, we hypothesize that regulatory B cells could additionally play a role in suppressed immunity following the dual immunotherapy combination in mPDAC. However, mechanistic studies need to be conducted to further understand these findings in the mPDAC setting.

Finally, the data presented in this study generate interesting hypotheses regarding the role of T cells in mediating immunity against pancreatic cancer and also suggests characterizing the immune state of the patient before treatment may help direct different immunotherapy-based treatment approaches. Prior to treatment, higher frequencies of circulating and infiltrating T cells and increased activated CD8 T cells, particularly with nivo/chemo treatment, were associated with improved survival. Notably, unlike data reported from other solid cancers³⁰⁻³³, circulating antigen experienced CD8 T cells or infiltrating CD8 T cells were not associated with survival suggesting other immune cell types are more prominent in mPDAC. In contrast, the associations with survival and circulating antigen experienced T cells were mainly observed with type-1 (Tbet+) CD4 T cells. Furthermore, infiltrating T cells in all tumor samples were largely CD4 T cells and surprisingly, very few patients' tumor samples had increased CD8 T cell infiltration (Supplementary Fig. X). Recent clinical studies have suggested that CD4 tumor infiltrating lymphocytes may be important for anti-tumor immunity³⁴. We hypothesize that the CD4 T cell compartment may have a critical role in response to chemoimmunotherapy treatment in mPDAC, a finding that has been yet to be reported in other solid cancer types.

This randomized Phase II trial is the first to suggest benefit of first-line chemoimmunotherapy in patients with mPDAC. A previous study of nivo/chemo failed to show clinical benefit in first-line mPDAC¹² patients; however, steroids were permitted in that study but steroid usage was discouraged in PRINCE. This study deployed multi-omic, multi-parameter biomarker analyses of unprecedented scale to identify potential mechanisms of response and resistance to chemoimmunotherapy regimens in mPDAC. Using this approach, we have identified novel biomarkers for these immune mechanisms in mPDAC. These multi-omic analyses revealed complex interplay of the TME and immune system consistent with previous preclinical work but have not been previously appreciated in patients with mPDAC. These results may aid in designing clinical trials and precision approaches for first-line nivo/chemo treatment and sotiga/chemo treatment in select patients with mPDAC. We plan to further assess the biomarker profiles presented herein in prospective biomarker-selected patient cohorts in upcoming clinical trials in this disease.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### REFERENCES

1. Rahib, L., et al. Projecting cancer incidence and deaths to 2030: the unexpected burden of thyroid, liver, and pancreas cancers in the United States. Cancer Res 74, 2913-2921 (2014).
2. Sharma, P., et al. The Next Decade of Immune Checkpoint Therapy. Cancer Discov 11, 838-857 (2021).
3. O'Reilly, E. M., et al. Durvalumab With or Without Tremelimumab for Patients With Metastatic Pancreatic Ductal Adenocarcinoma: A Phase 2 Randomized Clinical Trial. JAMA Oncol 5, 1431-1438 (2019).
4. Royal, R. E., et al. Phase 2 trial of single agent Ipilimumab (anti-CTLA-4) for locally advanced or metastatic pancreatic adenocarcinoma. J Immunother 33, 828-833 (2010).
5. Patnaik, A., et al. Phase I Study of Pembrolizumab (MK-3475; Anti-PD-1 Monoclonal Antibody) in Patients with Advanced Solid Tumors. Clin Cancer Res 21, 4286-4293 (2015).
6. Brahmer, J. R., et al. Safety and activity of anti-PD-L1 antibody in patients with advanced cancer. N Engl J Med 366, 2455-2465 (2012).
7. Balachandran, V. P., et al. Identification of unique neoantigen qualities in long-term survivors of pancreatic cancer. Nature 551, 512-516 (2017).
8. Balli, D., Rech, A. J., Stanger, B. Z. & Vonderheide, R. H. Immune Cytolytic Activity Stratifies Molecular Subsets of Human Pancreatic Cancer. Clin Cancer Res 23, 3129-3138 (2017).
9. Stromnes, I. M., Hulbert, A., Pierce, R. H., Greenberg, P. D. & Hingorani, S. R. T-cell Localization, Activation, and Clonal Expansion in Human Pancreatic Ductal Adenocarcinoma. Cancer Immunol Res 5, 978-991 (2017).
10. Byrne, K. T. & Vonderheide, R. H. CD40 Stimulation Obviates Innate Sensors and Drives T Cell Immunity in Cancer. Cell Rep 15, 2719-2732 (2016).
11. Winograd, R., et al. Induction of T-cell Immunity Overcomes Complete Resistance to PD-1 and CTLA-4 Blockade and Improves Survival in Pancreatic Carcinoma. Cancer Immunol Res 3, 399-411 (2015).
12. Wainberg, Z. A., et al. Open-label, Phase I Study of Nivolumab Combined with nab-Paclitaxel Plus Gemcitabine in Advanced Pancreatic Cancer. Clin Cancer Res 26, 4814-4822 (2020).
13. O'Hara, M. H., et al. CD40 agonistic monoclonal antibody APX005M (sotigalimab) and chemotherapy, with or without nivolumab, for the treatment of metastatic pancreatic adenocarcinoma: an open-label, multicentre, phase 1b study. Lancet Oncol 22, 118-131 (2021).
14. Von Hoff, D. D., et al. Increased survival in pancreatic cancer with nab-paclitaxel plus gemcitabine. N Engl J Med 369, 1691-1703 (2013).
15. Tempero, M., et al. Ibrutinib in combination with nab-paclitaxel and gemcitabine for first-line treatment of patients with metastatic pancreatic adenocarcinoma: phase III RESOLVE study. Ann Oncol 32, 600-608 (2021).
16. Van Cutsem, E., et al. Randomized Phase III Trial of Pegvorhyaluronidase Alfa With Nab-Paclitaxel Plus Gemcitabine for Patients With Hyaluronan-High Metastatic Pancreatic Adenocarcinoma. J Clin Oncol 38, 3185-3194 (2020).
17. Filbert, E. L., Bjorck, P. K., Srivastava, M. K., Bahjat, F. R. & Yang, X. APX005M, a CD40 agonist antibody with unique epitope specificity and Fc receptor binding profile for optimal therapeutic application. Cancer Immunol Immunother 70, 1853-1865 (2021).
18. Brahmer, J. R., et al. Phase I study of single-agent anti-programmed death-1 (MDX-1106) in refractory solid tumors: safety, clinical activity, pharmacodynamics, and immunologic correlates. J Clin Oncol 28, 3167-3175 (2010).
19. Noel, G., et al. Functional Th1-oriented T follicular helper cells that infiltrate human breast cancer promote effective adaptive immunity. J Clin Invest 131(2021).
20. Aoki, T., et al. Single-cell profiling reveals the importance of CXCL13/CXCR5 axis biology in lymphocyte-rich classic Hodgkin lymphoma. Proc Natl Acad Sci USA 118(2021).
21. Siska, P. J., Singer, K., Evert, K., Renner, K. & Kreutz, M. The immunological Warburg effect: Can a metabolic-tumor-stroma score (MeTS) guide cancer immunotherapy? Immunol Rev 295, 187-202 (2020).
22. Li, J., et al. Tumor Cell-Intrinsic Factors Underlie Heterogeneity of Immune Cell Infiltration and Response to Immunotherapy. Immunity 49, 178-193 c177 (2018).
23. Stebegg, M., et al. Regulation of the Germinal Center Response. Front Immunol 9, 2469 (2018).
24 Cohn, L., et al. Antigen delivery to early endosomes eliminates the superiority of human blood BDCA3+dendritic cells at cross presentation. J Exp Med 210, 1049-1063 (2013).
25. Haniffa, M., et al. Human tissues contain CD141hi cross-presenting dendritic cells with functional homology to mouse CD103+nonlymphoid dendritic cells. Immunity 37, 60-73 (2012).
26. Diamond, M. S., Lin, J. H. & Vonderheide, R. H. Site-Dependent Immune Escape Due to Impaired Dendritic Cell Cross-Priming. Cancer Immunol Res 9, 877-890 (2021).
27. Beltra, J. C., et al. Developmental Relationships of Four Exhausted CD8(+) T Cell Subsets Reveals Underlying Transcriptional and Epigenetic Landscape Control Mechanisms. Immunity 52, 825-841 c828 (2020).
28. Ding, C., et al. Integrin CD11b negatively regulates BCR signalling to maintain autoreactive B cell tolerance. Nat Commun 4, 2813 (2013).
29. van Hooren, L., et al. Agonistic CD40 therapy induces tertiary lymphoid structures but impairs responses to checkpoint blockade in glioma. Nat Commun 12, 4127 (2021).
30. Sharma, P., et al. CD8 tumor-infiltrating lymphocytes are predictive of survival in muscle-invasive urothelial carcinoma. Proc Natl Acad Sci USA 104, 3967-3972 (2007).
31. Ribas, A., et al. Oncolytic Virotherapy Promotes Intratumoral T Cell Infiltration and Improves Anti-PD-1 Immunotherapy. Cell 170, 1109-1119 e1110 (2017).
32. Tokito, T., et al. Predictive relevance of PD-L1 expression combined with CD8+ TIL density in stage III non-small cell lung cancer patients receiving concurrent chemoradiotherapy. Eur J Cancer 55, 7-14 (2016).
33. Yang, Z., et al. Tumor-Infiltrating PD-1(hi)CD8(+)-T-Cell Signature as an Effective Biomarker for Immune Checkpoint Inhibitor Therapy Response Across Multiple Cancers. Front Oncol 11, 695006 (2021).
34. Cachot, A., et al. Tumor-specific cytolytic CD4 T cells mediate immunity against human cancer. Sci Adv 7(2021).
35. Hartmann, F. J., et al. Comprehensive Immune Monitoring of Clinical Trials to Advance Human Immunotherapy. Cell Rep 28, 819-831 c814 (2019).
36. Spitzer, M. H., et al. IMMUNOLOGY. An interactive reference framework for modeling a dynamic immune system. Science 349, 1259425 (2015).
37. Assarsson, E., et al. Homogenous 96-plex PEA immunoassay exhibiting high sensitivity, specificity, and excellent scalability. PLOS One 9, c95192 (2014).
38. Marco Tognetti, K. S., Sebastian Müller, Dominique Kamber, Jan Muntel, Roland Bruderer, Lukas Reiter. Biomarker Candidates for Tumors Identified from Deep-Profiled Plasma Stem Predominantly from the Low Abundant Area. bioRxiv (2021).
39. Muntel, J., et al. Surpassing 10 000 identified and quantified proteins in a single run by optimizing current LC-MS instrumentation and data analysis strategy. Mol Omics 15, 348-360 (2019).
40. Patwardhan, A., et al. Achieving high-sensitivity for clinical applications using augmented exome sequencing. Genome Med 7, 71 (2015).
41. Dobin, A., et al. STAR: ultrafast universal RNA-seq aligner. Bioinformatics 29, 15-21 (2013).
42 DePristo, M. A., et al. A framework for variation discovery and genotyping using next-generation DNA sequencing data. Nat Genet 43, 491-498 (2011).
43. McKenna, A., et al. The Genome Analysis Toolkit: a MapReduce framework for analyzing next-generation DNA sequencing data. Genome Res 20, 1297-1303 (2010).
44. Lacey J Padrón, B. K., George Kierstein, Michael Travers, Marshall Thompson, Kurt Christensen, Robin Kageyama, Robert Schiemann, Daniel K Wells, Timothy Howes, Christine N Spencer, Nicholas Bayless, Stuart Halloway, Rich Hickey, Nikesh Kotecha, Pier Federico Gherardini. The CANDEL platform for reproducible biological data science. (In preparation).
45. R Development Core Team. R; A language and environment for statistical computing (R Foundation for Statistical Computing Vienna, Austria 2010).
46. Le Cao, K. A., Boitard, S. & Besse, P. Sparse PLS discriminant analysis: biologically relevant feature selection and graphical displays for multiclass problems. BMC Bioinformatics 12, 253 (2011).
47. Rohart, F., Gautier, B., Singh, A. & Le Cao, K. A. mixOmics: An R package for ‘omics feature selection and multiple data integration. PLOS Comput Biol 13, e1005752 (2017).

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

1-42. (canceled)

43. A method of treating a cancer in a human subject in need thereof, comprising:
(a) determining levels (cell counts) of at least one circulating peripheral blood mononuclear cell (PBMC) type selected from a group consisting of: HLA-DR+CCR7+B cells, cross-presenting dendritic cells (DCs), CD1C+CD141+cross-presenting DCs, PD-1+ T cells, TCF-1+ T cells, Tbet+ T cells, and T helper cells, in a biological sample from the subject; and
(b) administering a CD40 agonist in combination with a chemotherapeutic agent to the subject if the levels (cell counts) of at least one circulating PMBCs type select from a group consisting of: HLA-DR+CCR7+B cells, cross-presenting DCs, CD1C+CD141+cross-presenting DCs, PD-1+ T cells, TCF-1+ T cells, Tbet+ T cells, and T helper cells, are increased relative to a control or reference.

44. The method of claim 43, wherein:
(a) the subject is an individual among a cohort of subjects having the same cancer, wherein the subject is part of the cohort;
(b) the levels (cell counts) of at least one circulating PBMC type selected from a group consisting of: HLA-DR+CCR7+B cells, cross-presenting DCs, CD1C+CD141+cross-presenting DCs, PD-1+ T cells, TCF-1+ T cells, Tbet+ T cells, and T helper cells, are determined for each subject in the cohort; and
(c) the control or reference is calculated using the levels (cell counts) of the least one circulating PBMC type selected from a group consisting of: HLA-DR+CCR7+B cells, cross-presenting DCs, CD1C+CD141+cross-presenting DCs, PD-1+ T cells, TCF-1+ T cells, Tbet+ T cells, and T helper cells, of the subjects of the cohort.

45. The method of claim 43, wherein the circulating PBMC type is HLA-DR+CCR7+B cells.

46. The method of claim 43, wherein the circulating PBMC type is cross-presenting DCs.

47. The method of claim 43, wherein the circulating PBMC type CD1C+CD141+cross presenting DCs.

48. The method of claim 45, wherein the HLA-DR+CCR7+B cells are further screened based upon expression of CD38 and CD27.

49. The method of claim 48, wherein the HLA-DR+CCR7+B cells are plasmablasts.

50. The method of claim 43, wherein the circulating PBMC type is at least one of the following: PD-1+ T cells, TCF-1+ T cells, and Tbet+ T cells.

51. The method of claim 43, wherein the circulating PBMC type is PD-1+ T cells.

52. The method of claim 43, wherein the circulating PBMC type is TCF-1+ T cells.

53. The method of claim 43, wherein the circulating PBMC type is Tbet+ T cells.

54. A method of treating a cancer in a human subject in need thereof, comprising:
(a) determining levels (cell counts) of circulating 2B4+CD4 T cells in a biological sample from the subject; and
(b) administering a CD40 agonist in combination with a chemotherapeutic agent to the subject if the levels (cell counts) of circulating 2B4+CD4 T cells are decreased relative to a control or reference.

55. The method of claim 54, wherein:
(d) the subject is an individual among a cohort of subjects having the same cancer, wherein the subject is part of the cohort;
(e) the levels (cell counts) of circulating 2B4+CD4 T cells are determined for each subject in the cohort; and
(f) the control or reference is calculated using the levels (cell counts) of the circulating 2B4+CD4 T cells of the subjects of the cohort.

56. A method of treating a cancer in a human subject in need thereof, comprising:
(a) determining a gene signature in a biological sample from the subject, where in the gene signature is the MYC gene signature or the E2F gene signature, and calculating a signature score; and
(b) administering a CD40 agonist in combination with a chemotherapeutic agent to the subject if the gene signature score is decreased relative to a control or reference.

57. The method of claim 56, wherein:
(a) the subject is an individual among a cohort of subjects having the same cancer, wherein the subject is part of the cohort;
(b) the gene signature score of each subject in the cohort is calculated; and
(c) the control or reference is calculated using the gene signatures scores of the subjects of the cohort.

58. The method of claim 56, wherein the gene signature is the MYC gene signature.

59. The method of claim 58, wherein the MYC gene signature score is calculated by averaging log normalized expression values for each gene in a MYC gene set.

60. The method of claim 58, wherein the MYC gene set comprises one or more of genes known to be regulated by MYC version 1 (V1).

61. The method of claim 58, wherein the one or more genes are selected from the group consisting of tumor suppressor genes, oncogenes, translocated cancer genes, protein kinase genes, cell differentiation marker genes, homeodomain protein genes, transcription factor genes, cytokine genes, and growth factor genes.

62. The method of claim 58, wherein the one or more genes are selected from the group consisting of ABCE1, ACP1, AIMP2, AP3S1, APEX1, BUB3, C1QBP, CAD, CANX, CBX3, CCNA2, CCT2, CCT3, CCT4, CCT5, CCT7, CDC20, CDC45, CDK2, CDK4, CLNS1A, CNBP, COPS5, COX5A, CSTF2, CTPS1, CUL1, CYC1, DDX18, DDX21, DEK, DHX15, DUT, EEF1B2, EIF1AX, EIF2S1, EIF2S2, EIF3B, EIF3D, EIF3J, EIF4A1, EIF4E, EIF4G2, EIF4H, EPRS1, ERH, ETF1, EXOSC7, FAM120A, FBL, G3BP1, GLO1, GNL3, GOT2, GSPT1, H2AZ1, HDAC2, HDDC2, HDGF, HNRNPA1, HNRNPA2B1, HNRNPA3, HNRNPC, HNRNPD, HNRNPR, HNRNPU, HPRT1, HSP90AB1, HSPD1, HSPE1, IARS1, IFRD1, ILF2, IMPDH2, KARS1, KPNA2, KPNB1, LDHA, LSM2, LSM7, MAD2L1, MCM2, MCM4, MCM5, MCM6, MCM7, MRPL23, MRPL9, MRPS18B, MYC, NAP1L1, NCBP1, NCBP2, NDUFAB1, NHP2, NME1, NOLC1, NOP16, NOP56, NPM1, ODC1, ORC2, PA2G4, PABPC1, PABPC4, PCBP1, PCNA, PGK1, PHB, PHB2, POLD2, POLE3, PPIA, PPM1G, PRDX3, PRDX4, PRPF31, PRPS2, PSMA1, PSMA2, PSMA4, PSMA6, PSMA7, PSMB2, PSMB3, PSMC4, PSMC6, PSMD1, PSMD14, PSMD3, PSMD7, PSMD8, PTGES3, PWP1, RACK1, RAD23B, RAN, RANBP1, RFC4, RNPS1, RPL14, RPL18, RPL22, RPL34, RPL6, RPLP0, RPS10, RPS2, RPS3, RPS5, RPS6, RRM1, RRP9, RSL1D1, RUVBL2, SERBP1, SET, SF3A1, SF3B3, SLC25A3, SMARCC1, SNRPA, SNRPA1, SNRPB2, SNRPD1, SNRPD2, SNRPD3, SNRPG, SRM, SRPK1, SRSF1, SRSF2, SRSF3, SRSF7, SSB, SSBP1, STARD7, SYNCRIP, TARDBP, TCP1, TFDP1, TOMM70, TRA2B, TRIM28, TUFM, TXNL4A, TYMS, U2AF1, UBA2, UBE2E1, UBE2L3, USP1, VBP1, VDAC1, VDAC3, XPO1, XPOT, XRCC6, YWHAE, and YWHAQ.

63. The method of claim 56, wherein the gene signature is the E2F gene signature.

64. The method of claim 63, wherein the E2F gene signature score is calculated by averaging log normalized expression values for each gene in an E2F gene set.

65. The method of claim 63, wherein the E2F gene set comprises one or more genes selected from the group consisting of ABCE1, ACP1, AIMP2, AP3S1, APEX1, BUB3, C1QBP, CAD, CANX, CANX, CBX3, CCNA2, CCT2, CCT3, CCT4, CCT5, CCT7, CDC20, CDC45, CDK2, CDK4, CLNS1A, CNBP, COPS5, COX5A, CSTF2, CTPS1, CUL1, CYC1, DDX18, DDX21, DEK, DHX15, DUT, EEF1B2, EIF1AX, EIF2S1, EIF2S2, EIF3B, EIF3D, EIF3J, EIF4A1, EIF4E, EIF4G2, EIF4H, EPRS1, ERH, ETF1, EXOSC7, FAM120A, FBL, G3BP1, GLO1, GNL3, GOT2, GSPT1, H2AZ1, HDAC2, HDDC2, HDGF, HNRNPA1, HNRNPA2B1, HNRNPA3, HNRNPC, HNRNPD, HNRNPR, HNRNPU, HPRT1, HSP90AB1, HSPD1, HSPE1, IARS1, IFRD1, ILF2, IMPDH2, KARS1, KPNA2, KPNB1, LDHA, LSM2, LSM2, LSM7, MAD2L1 MCM2, MCM4, MCM5, MCM6, MCM7, MRPL23, MRPL23, MRPL9, MRPS18B, MYC, NAP1L1, NCBP1, NCBP2, NDUFAB1, NHP2, NME1, NOLC1, NOP16, NOP56, NPM1, ODC1, ORC2, PA2G4, PABPC1, PABPC4, PCBP1, PCNA, PGK1, PHB, PHB2, POLD2, POLE3, PPIA, PPM1G, PRDX3, PRDX4, PRPF31, PRPS2, PSMA1, PSMA2, PSMA4, PSMA6, PSMA7, PSMB2, PSMB3, PSMC4, PSMC4, PSMC6, PSMD1, PSMD14, PSMD3, PSMD7, PSMD8, PTGES3, PWP1, RACK1, RAD23B, RAN, RANBP1, RFC4, RNPS1, RPL14, RPL18, RPL22, RPL34, RPL6, RPLP0, RPS10, RPS2, RPS3, RPS5 RPS6, RRM1, RRP9, RSL1D1, RUVBL2, SERBP1, SET, SF3A1, SF3B3, SLC25A3, SMARCC1, SNRPA, SNRPA1, SNRPB2, SNRPD1, SNRPD2, SNRPD3, SNRPG, SRM, SRPK1, SRSF1, SRSF2, SRSF3, SRSF7, SSB, SSBP1, SSBP1, STARD7, SYNCRIP, TARDBP, TCP1, TFDP1, TOMM70, TRA2B, TRIM28, TUFM, TXNL4A, TYMS, U2AF1, UBA2, UBE2E1, UBE2L3, USP1, VBP1, VDAC1, VDAC3, XPO1, XPOT, XRCC6, YWHAE, YWHAE, and YWHAQ.
